(19) United States
(12) Patent Application Publication
(10) Pub. No.: US 2026/0285994 A1
(43) Pub. Date: Sep. 24, 2026

(54) CD40L-SPECIFIC TN3-DERIVED SCAFFOLDS FOR THE TREATMENT AND PREVENTION OF SJOGREN'S SYNDROME

(71) Applicant: Viela Bio, Inc., Deerfield, IL (US)

(72) Inventors: Ilias ALEVIZOS, Washington, DC (US); Gabor G. ILLEI, Rockville, MD (US); Yan XIN, San Carlos, CA (US); William A. REES, North Bethesda, MD (US); Donna MURRAY, Apex, NC (US); Kenneth R. DER, Danville, CA (US); Liangwei WANG, Deerfield, IL (US)

(73) Assignee: Viela Bio, Inc., Deerfield, IL (US)

(21) Appl. No.: 19/168,641

(22) PCT Filed: Mar. 27, 2024
(86) PCT No.: PCT/US2024/021716

§ 371 Date: Sep. 24, 2025

Related U.S. Application Data
(60) Provisional application No. 63/492,715, filed on Mar. 28, 2023.
(60) Provisional application No. 63/584,134, filed on Sep. 20, 2023.
(60) Provisional application No. 63/624,959, filed on Jan. 25, 2024.

(51) Int. Cl. C07K 16/28; A61K 38/00; A61P 37/06; C07K 14/765

(52) U.S. Cl. CPC C07K 16/2875; A61P 37/06; C07K 14/765; A61K 38/00; C07K 2318/20; C07K 2319/00

(57) ABSTRACT

Provided are compositions and methods comprising a CD40L-specific Tn3 scaffold. Also provided are methods of utilizing the same for the prevention and treatment of Sjögren's syndrome.

### CROSS REFERENCE TO RELATED APPLICATIONS

This application claims priority to U.S. Application Nos. 63/492,715, filed Mar. 28, 2023; 63/504,003, filed May 24, 2023; 63/584,134, filed Sep. 20, 2023; and 63/624,959 filed Jan. 25, 2024, each of which is incorporated by reference herein in its entirety for all purposes.

### REFERENCE TO AN ELECTRONIC SEQUENCE LISTING

The contents of the electronic sequence listing (HOPA_067_04WO_SeqList_ST26.xml; Size: 23,046 bytes; and Date of Creation: Feb. 16, 2024) are herein incorporated by reference in its entirety.

### TECHNICAL FIELD

The present disclosure is related to compositions comprising a Tn3 scaffold and methods using the same in the treatment and prevention of Sjögren's syndrome.

### BACKGROUND

Sjögren's syndrome (SS) is a systemic autoimmune disease characterized by chronic lymphocytic inflammation of the exocrine glands, mainly the salivary and lacrimal glands, leading to loss of function manifesting as excessive dryness. The subjective aspects of SS, which include the patient's perception of dryness, musculoskeletal pain, and fatigue can be debilitating and have been shown to have a substantial negative impact on quality of life (QoL). The major contributors to the decreased QOL are dryness and fatigue. QoL is also affected by psychological and emotional challenges and impaired social life with dependency on relatives in daily life and difficulties at work, as well as with other tasks.

Currently, there are no approved immunomodulating agents or evidence-based therapeutic guidelines available for treatment of the extra glandular manifestations of SS. There is thus a need for new treatments for Sjögren's syndrome.

### BRIEF DESCRIPTION OF THE DRAWINGS

FIG. 1 shows an exemplary study flow diagram. A=delta; D=day(s); Dazo=dazodalibep; EoS=End of Study; EP=endpoint; DASPRI=Diary for Assessing Sjögren's Patient Reported Index; ESSDAI=EULAR Sjögren's Syndrome Disease Activity Index; EULAR=European Alliance of Associations for Rheumatology; n=number of subjects in treatment group; PBO=placebo; q4wk=once every 4 weeks; q12wk=once every 12 weeks; wk=week. Note: Day 1 randomization stratified based on geographic location (North America and Europe versus Japan versus Rest of World), by coexisting RA or SLE (yes vs. no), and by screening ESSDAI score (<10 versus≥10).

FIG. 2 shows an exemplary study flow diagram. D=day(s); Dazo=dazodalibep; EP=endpoint; ESSPRI=EULAR Sjögren's Syndrome Patient Reported Index; EULAR=European Alliance of Associations for Rheumatology; n=number of subjects in treatment group; PBO=placebo; q4wk=once every 4 weeks; q12wk=once every 12 weeks; wk=week. Day 1 randomization stratified based on geographic location (North America and Europe versus Japan versus Rest of World) and screening ESSPRI score (<7.5 versus≥7.5).

FIG. 3 shows an exemplary study flow diagram. DAZ=dazodalibep; ESSDAI=EULAR Sjögren's Syndrome Disease Activity Index; EULAR=European League Against Rheumatism. Arrows indicate day of dosage for either dazodalibep or placebo.

FIG. 4 shows an exemplary change from baseline in ESSDAI Total Score. Changes from baseline in ESSDAI Total score were plotted as a function of time (days). Schedule of dazodalibep and placebo administration are described in FIG. 3. Analyzed using mixed model repeat measures (MMRM); *p<0.1; DAZ=dazodalibep; ESSDAI=EULAR Sjögren's Syndrome Disease Activity Index; LS=least-squares; MCID=minimal clinically important difference; SE=standard error.

FIG. 5 shows an exemplary change from baseline in ESSPRI Total Score. Changes from baseline in ESSPRI Total score were plotted as a function of time (days). Schedule of dazodalibep and placebo administration are described in FIG. 3. Analyzed using mixed model repeat measures (MMRM); DAZ=dazodalibep; ESSDAI=EULAR Sjögren's Syndrome Disease Activity Index; LS=least-squares; MCID=minimal clinically important difference; SE=standard error.

FIGS. 6A-6C show exemplary changes from baseline in FACIT-Fatigue score (FIG. 6A), OSDI (FIG. 6B), and PGIS (FIG. 6C). Changes from baseline were plotted as a function of time (days). Schedule of dazodalibep and placebo administration are described in FIG. 3.

FIGS. 7A-7G show exemplary changes in baseline for blood biomarkers of B and T cell co-stimulation as a function of dazodalibep or placebo administration over time (baseline to Day 365): CXCL13 (FIG. 7A); Rheumatoid Factor (RF; FIG. 7B); Ki67+ post-switch memory B cells (FIG. 7C); Plasmablasts (FIG. 7D); CD11cbr+ B cells (FIG. 7E); Ki67+ T cells (FIG. 7F); TfH Cells (FIG. 7G). Schedule of dazodalibep and placebo administration are described in FIG. 3. Data presented as median fold-change (FC) from baseline±IQR. Black symbols represent the placebo group (subjects receiving placebo in Stage I and dazodalibep in Stage II). Pink symbols represent the dazodalibep group (subjects receiving dazodalibep in Stage I and Placebo in Stage II). Statistical significance of differences in FC from baseline values between treatment groups was assessed using a Mann-Whitney U test. Subjects were only included in the analysis of RF if positive at baseline. *p<0.05; **p<0.01; ***p<0.001; DAZ=dazodalibep; FC=fold-change; IQR=interquartile range; PBO=placebo; RF=rheumatoid factor.

FIG. 8 shows an exemplary biomarker heatmap displaying median fold-change (FC) of baseline to Day 365 using biomarkers from FIGS. 7A-7G and ESSDAI total score. DAZ=dazodalibep; ESSDAI=EULAR Sjögren's Syndrome Disease Activity Index; PBO=placebo.

FIG. 9 shows an exemplary study flow diagram. DAZ=dazodalibep; ESSPRI=EULAR Sjögren's Syndrome Patient Reported Index; EULAR=European League Against Rheumatism. Arrows indicate day of dosage for either dazodalibep or placebo.

FIG. 10 shows an exemplary change from baseline in ESSPRI Total Score. Changes from baseline in ESSPRI Total score were plotted as a function of time (days). Schedule of dazodalibep and placebo administration are described in FIG. 9. Analyzed using mixed model repeat measures (MMRM); DAZ=dazodalibep; ESSDAI=EULAR Sjögren's Syndrome Disease Activity Index; LS=least-squares; MCID=minimal clinically important difference; SE=standard error.

FIGS. 11A-11C show exemplary changes in dryness (FIG. 11A), fatigue (FIG. 11B), and pain (FIG. 11C) from baseline. Changes from baseline were plotted as a function of time (days). Schedule of dazodalibep and placebo administration are described in FIG. 9. Data plotted by study visit; analyzed using MMRM. **p<0.01; BSL=baseline; DAZ=dazodalibep; LS=least-squares; PBO=placebo; SE=standard error.

FIG. 12 shows exemplary proportion of subjects achieving ESSPRI response for dazodalibep-treated subjects versus placebo through Day 169. ESSPRI response rate is plotted as a function of day. ESSPRI response is defined as ≥1 point or 15% reduction from baseline in ESSPRI score without premature discontinuation from the study and without receiving rescue therapy. Data were analyzed using a logistic regression model. ***p<0.001. CI=confidence interval. DAZ=dazodalibep. ESSPRI=EULAR Sjögren's Syndrome Patient Reported Index. EULAR=European Alliance of Associations for Rheumatology.

FIGS. 13A-13C show exemplary changes from baseline in FACIT-Fatigue score (FIG. 13A), OSDI (FIG. 13B), and PGIS (FIG. 13C). Changes from baseline were plotted as a function of time (days). Schedule of dazodalibep and placebo administration are described in FIG. 9.

### BRIEF SUMMARY

Provided herein are methods of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg once every 4 weeks, wherein the subject has moderate-to-severe symptom state defined by ESSPRI score of ≥5, with a low systemic disease activity defined by ESSDAI score of <5, and wherein a Diary for Assessing Sjögren's Patient Reported Index (DASPRI) score is reduced in the subject following the administration. In aspects, prior to the once every 4 week dosing, the subject was administered at least 3 loading doses of 1500 mg each. In aspects, the at least 3 loading doses are administered at weeks 0, 2, and 4.

Provided herein are methods of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 3000 mg once every 12 weeks, wherein the subject has moderate-to-severe symptom state defined by ESSPRI score≥5, with a low systemic disease activity defined by ESSDAI score<5, and wherein a Diary for Assessing Sjögren's Patient Reported Index (DASPRI) score is reduced in the subject following the administration. In aspects, prior to the once every 12 week dosing, the subject was administered at least 3 loading doses of 3000 mg each. In aspects, the at least 3 loading doses are administered at week 0, 4, and 12.

In aspects, salivary gland function in the subject is improved following administration of the Tn3 scaffold by about 3 weeks, 1 month, 2 months, or 4 months following the administration. In aspects, salivary gland function is determined by whole stimulated salivary flow. In aspects, the ESSPRI score is reduced following the administration. In aspects, the ESSPRI score is reduced by at least about 1.5, 2, 3, 4, or 5 points. In aspects, the DASPRI score is reduced by 2 weeks, 4 weeks, 1 month, 2 months, 3 months, 4 months, 5 months, or 6 months following the administration. In aspects, the DASPRI score is reduced by at least about 5, 10, 15, or 20 points. In aspects, the administration of the Tn3 scaffold is effective at reducing a tender and swollen joint count baseline score of the subject following the administration.

In aspects, the administration of the Tn3 scaffold is effective at reducing a Short Form 36 (SF-36) Health Survey baseline score of the subject following the administration. In aspects, the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS). In aspects, the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof. In aspects, the administration of the Tn3 scaffold is effective at reducing a baseline level of a biomarker, and wherein the biomarker is selected from the group consisting of: plasma soluble CD40L, Ki67+CD27+ memory B cell, CD19, CD20, CD27, CD38, CD138, CD11c bright/high B cell, CD3, CD4, CD8, T follicular helper (Tfh) cell, serum CXCL13, rheumatoid factor, anti-SSA autoantibodies, anti-Ro autoantibodies, anti-SSB autoantibodies, anti-La autoantibodies, and combinations thereof. In aspects, the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof. In aspects, expression levels of genes, or levels of proteins encoded by the genes, associated with SS are reduced in a blood sample of the subject by week 48 following the administration. In aspects, levels of B cells selected from the group consisting of: CD27+ memory B cells, marginal zone B cells, plasmablasts, plasma cells, and combinations thereof, are reduced in a blood sample of the subject by week 48 following the administration. In aspects, the subject is positive for anti-Ro autoantibodies, rheumatoid factor (RF), or both anti-Ro autoantibodies and RF. In aspects, the Tn3 scaffold is administered intravenously.

In methods disclosed herein, a Tn3 scaffold comprises two CD40L-specific monomer subunits connected in tandem. In aspects, the two CD40L-specific monomer subunits each comprise SEQ ID NO: 3. In aspects, the CD40L-specific monomer subunits are connected by a linker. In aspects, at least one CD40L-specific monomer subunit is fused or conjugated to a polyethylene glycol (PEG) directly. In aspects, at least one CD40L-specific monomer subunit is fused or conjugated to a polyethylene glycol (PEG) via a linker. In aspects, the linker comprises a peptide linker. In aspects, the linker comprises SEQ ID NO: 7, SEQ ID NO: 8, SEQ ID NO: 9, or SEQ ID NO: 10. In aspects, at least one CD40L-specific monomer subunit is fused or conjugated to an albumin. In aspects, the albumin is human serum albumin (HSA). In aspects, the HSA is a variant HSA comprising SEQ ID NO: 4. In aspects, the Tn3 scaffold comprises SEQ ID NO: 1.

In aspects, a subject of the disclosure has a co-existing autoimmune indication. In aspects, the co-existing autoimmune indication is rheumatoid arthritis (RA), systemic lupus erythematosus (SLE), or both RA and SLE. In aspects, the co-existing autoimmune indication is rheumatoid arthritis. In aspects, the co-existing autoimmune indication is systemic lupus erythematosus.

### BRIEF DESCRIPTION

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. In aspects, the subject is a mammal. A mammal includes primates, such as humans, monkeys, chimpanzee, and apes, and non-primates such as domestic animals.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, “fused” refers to at least two polypeptides joined recombinantly. As used herein, “conjugated” refers to formation of a bond between two components by chemical reaction. Typically, two components that are conjugated to each other are chemically connected via a covalent bond.

As used herein, “treating” or “treat” describes the management and care of a subject for the purpose of combating a disease, condition, or disorder and includes the administration of a Tn3 scaffold used in the methods described herein to alleviate the symptoms or complications of a disease, condition or disorder, or to eliminate the disease, condition or disorder. Thus, the term “treat” or “treating” refers to therapeutic wherein the objective is to slow down (lessen) or ameliorate the progression of a disease (e.g., an autoimmune disease or disorder). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total).

As used herein, an “effective dose” describes a quantity of a Tn3 scaffold used in the methods described herein that reduces or eliminates one or more symptoms of a disease, condition, or disorder.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### Tn3 Scaffolds

Provided herein are compositions that bind CD40L. In aspects, provided compositions comprise CD40L antagonists. In aspects, provided herein are compositions that comprise a Tn3 scaffold protein comprising a CD40L-specific monomer subunit (e.g., “Tn3 scaffold”). In aspects, provided herein are compositions that comprise a Tn3 scaffold comprising two CD40L-specific monomer subunits. In aspects, a CD40L-specific monomer subunit is also known as a CD40L-specific Tn3 monomer. The term “Tn3 scaffold” used herein, refers to molecules comprising at least one Fnlll scaffold wherein the A beta strand comprises SEQ ID NO: 5, 23, or 24, the B beta strand comprises SEQ ID NO: 6, the C beta strand SEQ ID NO: 17, the D beta strand comprises SEQ ID NO: 18, the E beta strand comprises SEQ ID NO: 19, the F beta strand comprises SEQ ID NO: 20, and the beta strand G comprises SEQ ID NO: 21.

In aspects, provided compositions may comprise any of the Tn3 scaffolds having the amino acid sequences as described in Int'l Appl. Nos. PCT/US2012/059477 and PCT/US2019/052997, which are incorporated herein by reference in their entireties. In aspects, provided compositions may comprise a Tn3 scaffold having the amino acid sequence as shown in SEQ ID NO: 1 (referred to herein as Dazodalibep). Dazodalibep may also be referred to a VIB4920, MEDI4920, or HZN-4920.

In aspects, a CD40L monomer subunit of a Tn3 scaffold comprises seven beta strands designated A, B, C, D, E, F, and G, and six loop regions designated AB, BC, CD, DE, EF, and FG. In aspects, the Tn3 scaffold comprises a single CD40L-specific monomer subunit. In aspects, the Tn3 scaffold comprises two CD40L-specific monomer subunits. In aspects, the two CD40L-specific monomer subunits are connected in tandem. In aspects, the two CD40L-specific monomer subunits are connected by a linker. In aspects, the linker comprises a peptide linker, which can be a flexible peptide linker. In aspects, the peptide linker comprises a (GmX) n sequence wherein X is Serine(S), Alanine (A), Glycine (G), Leu (L), Isoleucine (I), or Valine (V); m and n are integer values; m is 1, 2, 3 or 4; and n is 1, 2, 3, 4, 5, 6, or 7.

In aspects, the Tn3 scaffold comprises a linker which comprises a functional moiety. In aspects, this functional moiety is an immunoglobulin or a fragment thereof. In aspects, this immunoglobulin or fragment thereof comprises an Fc domain. In aspects, this Fc domain fails to induce at least one FcγR-mediated effector function (e.g., Fc-deficient). In aspects, this at least one FcγR-mediated effector function is antibody-dependent cellular cytotoxicity (ADCC).

In aspects, the Tn3 scaffold comprises a CD40L-specific monomer subunit comprising seven beta strands designated A, B, C, D, E, F, and G, and six loop regions designated AB, BC, CD, DE, EF, and FG, wherein the AB loop comprises or consists of SEQ ID NO: 11, the BC loop comprises or consists of SEQ ID NO: 12, the CD loop comprises or consists of SEQ ID NO: 13, the DE loop comprises or consists of SEQ ID NO: 14, the EF loop comprises or consists of SEQ ID NO: 15, and the FG loop comprises or consists of SEQ ID NO: 16. In aspects, the Tn3 scaffold comprises or consists of SEQ ID NO: 1 (also known as VIB4920). In aspects, beta strand A comprises or consists of SEQ ID NO: 5, beta strand B comprises or consists of SEQ ID NO: 6, beta strand C comprises or consists of SEQ ID NO: 17, beta strand D comprises or consists of SEQ ID NO: 18, beta strand E comprises or consists of SEQ ID NO: 19, beta strand F comprises or consists of SEQ ID NO: 20, and beta strand G comprises or consists of SEQ ID NO: 21.

In aspects, one or more CD40L-specific Tn3 monomers have a beta strand A comprising or consisting of IEV (SEQ ID NO: 5), RLDAPSQIEV (SEQ ID NO: 23), or SQIEV (SEQ ID NO: 24). In aspects, a Tn3 scaffold may comprise one or more CD40L-specific Tn3 monomers having the same or different beta strand A sequences. For example, a first CD40L-specific Tn3 monomer beta strand A may comprise or consist of IEV (SEQ ID NO: 5) and a second CD40L-specific Tn3 monomer beta strand A may comprise or consist of RLDAPSQIEV (SEQ ID NO: 23) or SQIEV (SEQ ID NO: 24).

The Tn3 scaffold may have the amino acid sequence as shown in SEQ ID NO: 1 and described above or it may have one or more amino acid residues changes relative to the amino acid sequence as shown in SEQ ID NO: 1. For example, if the scaffold has amino acid sequence changes relative to those shown in SEQ ID NO: 1, the changes may be to one of the linkers. The Tn3 scaffold may comprise a Glyl5 linker separating two CD40L-specific monomers and a Glyl0 linker separating a CD40L-specific monomer from an HSA sequence. Both or one of these linkers may be altered, and may be replaced with an amino acid sequence of (GmX) n wherein X is Serine(S), Alanine (A), Glycine (G), Leu (L), Isoleucine (I), or Valine (V); m and n are integer values; m is 1, 2, 3 or 4; and, n is 1, 2, 3, 4, 5, 6, or 7. For example, one or both linkers may be altered to have an amino acid sequence that comprises one of GGGGSGGGGS (SEQ ID NO: 7), GGGGSGGGGSGGGGS (SEQ ID NO: 8), GGGGGGGGGG (SEQ ID NO: 9) or GGGGGGGGGGGGGGG (SEQ ID NO: 10). If the Tn3 scaffold has an amino acid sequence relative to the amino acid sequence as provided in SEQ ID NO: 1, it may be due to a changes or changes in the HSA amino acid sequence fused to the two CD40L-specific monomers. The HSA fused to the two CD40L-specific monomers may be altered to relative to the HSA fused to the two CD40L-specific Tn3 monomers, except for at least one amino acid substitution, numbered relative to the position in full length mature HSA, at a position selected from the group consisting of 407, 415, 463, 500, 506, 508, 509, 511, 512, 515, 516, 521, 523, 524, 526, 535, 550, 557, 573, 574, and 580; wherein the at least one amino acid substitution does not comprise a lysine (K) to glutamic acid (E) at position 573.

Exemplary sequences for a Tn3 scaffolds are shown in Table 1. In aspects, a Tn3 scaffold comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 25 shown in Table 1. In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 1-3, 1-5, 1-10, 5-20, 1-3, 1-5, 1-10, 1-20, 3-8, 3-10, 3-15, 5-8, 5-10, or 5-20 residues. In aspects, a modification can occur in up to 1, 2, 3, 4, 5, 6, 7, 8, 9, 10, 11, 12, 13, 14, 15, 20, 30, 40, 50, 60, 70, 80, 90, 100, 200, 300, 400, or 450 residues.

TABLE 1
Exemplary sequences for a Tn3 scaffold comprising a CD40L-specific monomer subunit

| SEQ | | |
|---|---|---|
| ID NO | ID | Sequence |
| 1 | VIB4920 | SQIEVKDVTDTTALITWSDDFGEYVWCELTYGIKDVPGDRTTI |
| | (342-G15-342- | DLWYHHAHYSIGNLKPDTEYEVSLICRSGDMSSNPAKETFTT |
| | G10-HSAC34S | GGGGGGGGGGGGGGGRLDAPSQIEVKDVTDTTALITWSDDF |
| | Bivalent | GEYVWCELTYGIKDVPGDRTTIDLWYHHAHYSIGNLKPDTEY |
| | construct 2- All | EVSLICRSGDMSSNPAKETFTTGGGGGGGGGGDAHKSEVAHR |
| | GLY linkers | FKDLGEENFKALVLIAFAQYLQQSPFEDHVKLVNEVTEFAKT |
| | HSA | CVADESAENCDKSLHTLFGDKLCTVATLRETYGEMADCCAK |
| | underlined) | QEPERNECFLQHKDDNPNLPRLVRPEVDVMCTAFHDNEETFL |
| | | KKYLYEIARRHPYFYAPELLFFAKRYKAAFTECCQAADKAAC |
| | | LLPKLDELRDEGKASSAKQRLKCASLQKFGERAFKAWAVAR |
| | | LSQRFPKAEFAEVSKLVTDLTKVHTECCHGDLLECADDRADL |
| | | AKYICENQDSISSKLKECCEKPLLEKSHCIAEVENDEMPADLPS |
| | | LAADFVESKDVCKNYAEAKDVFLGMFLYEYARRHPDYSVVL |
| | | LLRLAKTYETTLEKCCAAADPHECYAKVFDEFKPLVEEPQNLI |
| | | KQNCELFEQLGEYKFQNALLVRYTKKVPQVSTPTLVEVSRNL |
| | | GKVGSKCCKHPEAKRMPCAEDYLSVVLNQLCVLHEKTPVSD |
| | | RVTKCCTESLVNRRPCFSALEVDETYVPKEFNAETFTFHADIC |
| | | TLSEKERQIKKQTALVELVKHKPKATKEQLKAVMDDFAAFVE |
| | | KCCKADDKETCFAEEGKKLVAASQAALGL |
| 2 | CD40L-specific | IEVKDVTDTTALITWSDDFGEYVWCELTYGIKDVPGDRTTIDL |
| | Tn3 monomer | WYHHAHYSIGNLKPDTEYEVSLICRRGDMSSNPAKETFTT |
| | with affinity | |
| | mature variant | |
| | Clone 342- | |
| | Affinity mature | |
| | variant | |
| | (W/WT FG | |
| | loop; W/O N- | |
| | Term A, C- | |
| | Term Linker and | |
| | His8Tag | |
| 3 | CD40L-specific | IEVKDVTDTTALITWSDDFGEYVWCELTYGIKDVPGDRTTIDL |
| | Tn3 monomer | WYHHAHYSIGNLKPDTEYEVSLICRSGDMSSNPAKETFTT |
| | with affinity | |
| | mature variant | |
| | Clone 342- | |
| | Affinity mature | |
| | variant | |
| | (W/FG loop | |
| | variant RR → RS | |
| | underlined) | |
| 4 | Human serum | DAHKSEVAHRFKDLGEENFKALVLIAFAQYLQQSPFEDHVKL |
| | albumin variant | VNEVTEFAKTCVADESAENCDKSLHTLFGDKLCTVATLRETY |
| | | GEMADCCAKQEPERNECFLQHKDDNPNLPRLVRPEVDVMCT |
| | | AFHDNEETFLKKYLYEIARRHPYFYAPELLFFAKRYKAAFTEC |
| | | CQAADKAACLLPKLDELRDEGKASSAKQRLKCASLQKFGER |
| | | AFKAWAVARLSQRFPKAEFAEVSKLVTDLTKVHTECCHGDL |
| | | LECADDRADLAKYICENQDSISSKLKECCEKPLLEKSHCIAEV |
| | | ENDEMPADLPSLAADFVESKDVCKNYAEAKDVFLGMFLYEY |
| | | ARRHPDYSVVLLLRLAKTYETTLEKCCAAADPHECYAKVFDE |
| | | FKPLVEEPQNLIKQNCELFEQLGEYKFQNALLVRYTKKVPQVS |
| | | TPTLVEVSRNLGKVGSKCCKHPEAKRMPCAEDYLSVVLNQLC |
| | | VLHEKTPVSDRVTKCCTESLVNRRPCFSALEVDETYVPKEFNA |
| | | ETFTFHADICTLSEKERQIKKQTALVELVKHKPKATKEQLKAV |
| | | MDDFAAFVEKCCKADDKETCFAEEGKKLVAASQAALGL |
| 5 | beta strand “A” | IEV |
| | within a | |
| | CD40L-specific | |
| | monomer | |
| 6 | beta strand “B” | ALITW |
| | within a | |
| | CD40L-specific | |
| | monomer | |
| 7 | Linker | GGGGSGGGGS |
| 8 | Linker | GGGGSGGGGSGGGGS |
| 9 | Linker | GGGGGGGGGG |
| 10 | Linker | GGGGGGGGGGGGGGG |
| 11 | AB loop | KDVTDTT |
| 12 | BC loop | SDDFGEYVW |
| 13 | CD loop | KDVPGDR |
| 14 | DE loop | WYHHAH |
| 15 | EF loop | GNLKPDTE |
| 16 | FG loop | RSGDMSSNPA |
| 17 | beta strand “C” | CELTYGI |
| | within a | |
| | CD40L-specific | |
| | monomer | |
| 18 | beta strand “D” | TTIDL |
| | within a | |
| | CD40L-specific | |
| | monomer | |
| 19 | beta strand “E” | YSI |
| | within a | |
| | CD40L-specific | |
| | monomer | |
| 20 | beta strand “F” | YEVSLIC |
| | within a | |
| | CD40L-specific | |
| | monomer | |
| 21 | beta strand “G” | KETFTT |
| | within a | |
| | CD40L-specific | |
| | monomer | |
| 22 | CD40L-specific | SQIEVKDVTDTTALITWSDDFGEYVWCELTYGIKDVPGDRTTI |
| | Tn3 monomer | DLWYHHAHYSIGNLKPDTEYEVSLICRSGDMSSNPAKETFTT |
| | Clone 342- | |
| | Affinity mature | |
| | variant | |
| | (W/FG loop | |
| | variant RR → RS | |
| | underlined) | |
| 23 | beta strand | RLDAPSQIEV |
| | sequence “A” | |
| | within a | |
| | CD40L-specific | |
| | monomer | |
| 24 | beta strand “A” | SQIEV |
| | within a | |
| | CD40L-specific | |
| | monomer | |
| 25 | CD40L-specific | RLDAPSQIEVKDVTDTTALITWSDDFGEYVWCELTYGIKDVP |
| | Tn3 monomer | GDRTTIDLWYHHAHYSIGNLKPDTEYEVSLICRSGDMSSNPAK |
| | | ETFTT |

If the Tn3 scaffold has amino acid sequence changes relative to those shown in SEQ ID NO: 1, the changes may be to the amino acid sequence of one or both of the CD40L-specific Tn3 monomers, so long as it does not adversely effect in vivo efficacy of the scaffold, e.g., change in amino acid sequence such that one or both CD40L-specific Tn3 monomers have the amino acid sequence as shown in SEQ ID NO: 2, SEQ ID NO: 3, SEQ ID NO: 22, and SEQ ID NO: 25. In aspects, the first one or two N-terminal amino acid residues (SQ) may be absent and/or substituted with alternative amino acid residues. In aspects, a Tn3 scaffold comprises a monomer subunit comprising SEQ ID NO: 22, SEQ ID NO: 25, or both SEQ ID NO: 22 and SEQ ID NO: 25.

In aspects, a Tn3 scaffold comprises at least one CD40L-specific monomer subunit bound to a heterologous moiety. In aspects, this heterologous moiety is selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a HSA FcRn binding portion, an antibody or fragment thereof, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of said moieties. In aspects, the heterologous moiety is the albumin, and the albumin comprises human serum albumin. In aspects, the heterologous moiety is an antibody. In aspects, the antibody is selected from the group consisting of: an Fc domain of an antibody, an antibody fragment, and a single chain antibody.

In aspects, the heterologous moiety is an antibody. In aspects, the antibody is selected from the group consisting of: an Fc domain of an antibody, an antibody fragment, and a single chain antibody.

In aspects, the heterologous moiety is an imaging agent; for example, a radionuclide or biotin. In aspects, the heterologous moiety is a drug; for example, a cytotoxic agent or a radioactive compound.

In aspects, the heterologous moiety comprises PEG. In aspects, the Tn3 scaffold comprises at least one CD40L-specific monomer subunit fused or conjugated directly or via a linker to PEG. In aspects, both CD40L-specific monomer subunits are fused, conjugated, or connected via a linker to PEG. In aspects, the Tn3 scaffold comprises at least one (e.g., two) CD40L-specific monomer subunit fused or conjugated directly or via a linker to PEG.

In aspects, the heterologous moiety comprises albumin. In aspects, the Tn3 scaffold comprises at least one CD40L-specific monomer subunit fused or conjugated directly or via a linker to an albumin. In aspects, this albumin is HSA. In aspects, this HSA is a variant HSA. In aspects, the amino acid sequence of the variant HSA is SEQ ID NO: 4. In aspects, the variant HSA has at least one improved property compared with a native HSA or a native HSA fragment. In aspects, the amino acid sequence of the variant HSA is SEQ ID NO: 4 or a sequence having at least about 80%, 85%, 90%, 95%, 96%, 97%, 98%, 99%, or up to 100% identity to SEQ ID NO: 4. In aspects, the improved property is an altered plasma half-life compared with the plasma half-life of a native HSA or a native HSA fragment. In aspects, the altered plasma half-life is a longer plasma half-life compared with the plasma half-life of a native HSA or a native HSA fragment. In aspects, the altered plasma half-life is a shorter plasma half-life compared with the plasma half-life of a native HSA or a native HSA fragment.

### Dosing

In aspects, any of the compositions comprising a Tn3 scaffold of the disclosure can be administered in any form. In aspects, a Tn3 scaffold is administered intravenously, subcutaneously, orally, intramuscularly, intrathecally, sublingually, rectally, vaginally, cutaneously, systemically, topically, transdermally, or by way of inhalation. In aspects, the Tn3 scaffold is administered intravenously. In aspects, the Tn3 scaffold is administered by intravenous infusion.

A Tn3 scaffold of the disclosure can be administered at any dose. In aspects, a Tn3 scaffold is administered at a dose from about and/or up to about: 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, or about 5000 mg. Any of the aforementioned dosages may be effective dosages for a method comprising treatment, reduction, or elimination.

In aspects, a Tn3 scaffold is administered at a dose of between about: 800-5000 mg, 900-4900 mg, 1000-4800 mg, 1100-4700 mg, 1200-4600 mg, 1300-4500 mg, or 1500-3000 mg. In aspects, a Tn3 scaffold is administered at a dose selected from the group consisting of: 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, and 4500 mg. In aspects, a Tn3 scaffold is administered at a dose of between about 1500 mg and 3000 mg. In aspects, a Tn3 scaffold is administered at a dose of: 1500 mg or 3000 mg. In aspects, a Tn3 scaffold is administered at a dose of about 1500 mg. In aspects, a Tn3 scaffold is administered at a dose of about 3000 mg.

### Dosing Frequency

In aspects, a Tn3 scaffold of the disclosure is administered on a schedule that provides optimal results. In aspects, a Tn3 scaffold is administered to a subject in need thereof about once a week, about twice a week, about every two weeks, about once a month, about every four weeks, about every two months, about every 3 months, about every 12 weeks, about every fifteen weeks, about every sixteen weeks, about every four months, about every five months, about every six months, or semiannually. Any number of administrations may be provided to a subject in need thereof. In aspects, a Tn3 scaffold of the disclosure is administered as a loading dose. A loading does may also be known as an induction dose. In aspects, a Tn3 scaffold is administered as a maintenance dose.

A Tn3 scaffold of the disclosure can be administered from about 1-10, 10-50, 50-75, 75-100, 100-200, or 200-300 total doses, or up to the lifetime of a subject. In aspects, a Tn3 scaffold is administered as about 1, 2, 3, 4, 5, 6, 7, 8, 9, 10, 11, 12, 13, 14, 15, 16, 17, 18, 19, 20, 25, 30 or more doses. In aspects, a Tn3 scaffold is administered at least about or at most about 2, 3, 4, or 5 total doses. In aspects, a Tn3 scaffold is administered at least about or at most about 10, 11, 12, or 13 total doses.

In aspects, a subject is administered an effective dose on about every 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 1 year, 2 years, 3 years, 4 years, or 5 years, or up to the lifetime of a subject, post treatment initiation. In aspects, a subject receives an effective dose on Day 1, Day 15, Day 29, Day 57, Day 85, day 113, Day 141, Day 169, Day 197, Day 225, Day 253, Day 281, and Day 309 post treatment initiation. In aspects, a subject receives 1500 mg-3000 mg of a Tn3 scaffold on Day 1, Day 15, Day 29, Day 57, Day 85, day 113, Day 141, Day 169, Day 197, Day 225, Day 253, Day 281, and Day 309 post treatment initiation.

In aspects, a subject is administered from about 1500 mg-3000 mg of a Tn3 scaffold every 2 weeks for about 3 administrations then every 4 weeks thereafter. In aspects, a subject is administered about 1500 mg of a Tn3 scaffold every 2 weeks for about 3 administrations then every 4 weeks thereafter. In aspects, a subject is administered about 3000 mg of a Tn3 scaffold at week 0, 4, and 12 then every 12 weeks thereafter. In aspects, a subject is administered about 1500 mg of a Tn3 scaffold every 4 weeks. In aspects, a subject is administered about 3000 mg of a Tn3 scaffold every 12 weeks. In aspects, a subject is administered an initial dose of a Tn3 scaffold of the disclosure of about 1500 mg-3000 mg every 2 weeks for at least 2, at least 3, or more administrations, and then every 4 weeks thereafter. In aspects, a subject is administered an initial dose of a Tn3 scaffold of the disclosure of about 1500 mg every 2 weeks for at least 2, at least 3, or more administrations, and then every 4 weeks thereafter. In aspects, a subject is administered an initial dose of a Tn3 scaffold of the disclosure of about 3000 mg every 2 weeks for at least 2, at least 3, or more administrations, and then every 12 weeks thereafter. In aspects, a subject is administered an initial dose of a Tn3 scaffold of the disclosure of about 3000 mg every 4 weeks for at least 2, at least 3, or more administrations, and then every 12 weeks thereafter. In aspects, the administration treats a subject with SS with low systemic disease activity, but moderate to severe symptom state. In aspects, the administration treats a subject with SS with moderate systemic disease activity. In aspects, the administration treats a subject with SS with severe systemic disease activity. In aspects, the administration treats a subject with SS with moderate to severe systemic disease activity. In aspects, administration of a Tn3 scaffold treats a subject with SS with moderate or severe systemic disease activity as defined by an ESSDAI score≥5. In aspects, administration of a Tn3 scaffold treats a subject with SS with moderate to severe systemic disease activity as defined by an ESSDAI score≥5. In aspects, administration of a Tn3 scaffold treats a subject with SS with moderate to severe symptomatic activity as defined an ESSPRI score of ≥5 and low systemic disease activity with ESSDAI score<5. In aspects, administration of a Tn3 scaffold treats a subject with SS with moderate or severe systemic disease activity as defined by an ESSDAI score≥5. In aspects, administration of a Tn3 scaffold treats a subject with SS with moderate or severe systemic disease activity as defined by an ESSDAI score≥5. In aspects, administration of a Tn3 scaffold treats a subject with SS with moderate or severe symptomatic activity as defined an ESSPRI score of ≥5 and low systemic disease activity with ESSDAI score<5.

Methods of treatment or prevention of the disclosure comprise administering a Tn3 scaffold of the disclosure to a subject in need thereof. In aspects, a method of treatment comprises administering an effective dose of a Tn3 scaffold to a subject in need every 2 weeks for about 3 doses, followed by an administration of a Tn3 scaffold every 4 weeks thereafter. In aspects, a method of treatment comprises administering an effective dose of a Tn3 scaffold to a subject in need on week 0, 4, and 12, followed by an administration of a Tn3 scaffold every 12 weeks thereafter. In aspects, a method of treatment comprises administering from about 1400 mg to about 1600 mg of a Tn3 scaffold to a subject in need every 2 weeks for about 3 administrations, followed by an administration of a Tn3 scaffold every 4 weeks thereafter. In aspects, a method of treatment comprises administering from about 2000 mg to about 4000 mg of a Tn3 scaffold to a subject in need on week 0, 4, and 12, followed by an administration of a Tn3 scaffold every 12 weeks thereafter.

In aspects, a method of treatment comprises administering from about 1500 mg of a Tn3 scaffold to a subject in need every 2 weeks for 7 administrations. In aspects, a method of treatment comprises administering from about 1500 mg of a Tn3 scaffold to a subject in need every 4 weeks for 5 administrations. In aspects, a method of treatment comprises administering from about 1500 mg of a Tn3 scaffold to a subject in need every 2 weeks for about 3 administrations, followed by an administration of the Tn3 scaffold every 4 weeks thereafter. In aspects, a method of treatment comprises administering from about 3000 mg of a Tn3 scaffold to a subject in need on week 0, 4, and 12, followed by an administration of the Tn3 scaffold every 12 weeks thereafter. In aspects, administrations continue every 4 weeks thereafter up to about 1 year, 2 years, 3 years, 4 years, or 5 years, or up to the lifetime of a subject. In aspects, any of the aforementioned administrations can deviate by about 1 day to about 4 days, about 3 days to about 7 days, or by about 1 day to about 7 days. In aspects, any of the aforementioned administrations can deviate by about 1 day, 3 days, 4 days, or 7 days. In aspects, any of the aforementioned administrations can deviate by about 1 day. In aspects, any of the aforementioned administrations can deviate by about 3 days. In aspects, any of the aforementioned administrations can deviate by about 4 days. In aspects, any of the aforementioned administrations can deviate by about 7 days.

In aspects, a subject receives an effective dose of a Tn3 scaffold on Day 1, Day 15 (−3 days to ±1 day), Day 29 (±4 days), and Day 57 (±7 days) post treatment initiation. In aspects, a subject in need thereof is administered 1500 mg of a Tn3 scaffold on Day 1, Day 15 (−3 days to +1 day), Day 29 (±4 days), and Day 57 (±7 days) post treatment initiation, and then every 4 weeks thereafter as needed. In aspects, a subject in need thereof is administered 1500 mg of a Tn3 scaffold on Day 1, Day 15 (±1 day), and Day 29 (±3 days) post treatment initiation. In aspects, a subject in need thereof is administered 1500 mg of a Tn3 scaffold on Day 1 and Day 57 (±7 days) post treatment initiation. In aspects, a subject in need thereof is administered 1500 mg of a Tn3 scaffold on Day 1, Day 15 (±1 day), Day 29 (±3 days), Day 57 (±7 days), Day 85 (±7 days), Day 113 (±7 days), Day 141 (±7 days) post treatment initiation. In aspects, a subject in need thereof is administered 1500 mg of a Tn3 scaffold on Day 169 (±7 days), Day 197 (±7 days), Day 225 (±7 days), Day 253 (±7 days), and Day 281 (±7 days) post treatment initiation. In aspects, a subject in need thereof is administered 3000 mg of a Tn3 scaffold on Day 1, Day 15 (−3 days to +1 day), Day 29 (±4 days), and Day 57 (±7 days) post treatment initiation, and then every 6 months thereafter as needed. In aspects, a subject in need thereof is administered 3000 mg of a Tn3 scaffold on Day 1, Day 15 (−3 days to +1 day), and Day 29 (±4 days) post treatment initiation. In aspects, a subject in need thereof is administered 3000 mg of a Tn3 scaffold on Day 1 and Day 57 (±7 days) post treatment initiation, and then every 12 weeks thereafter as needed.

In aspects, a subject in need thereof receives a dose of a Tn3 scaffold on Day 1, Day 15 (−3 days to +1 day), Day 29 (±4 days), Day 57 (±7 days), Day 85 (±7 days), Day 113 (±7 days), and Day 141 (±7 days). In aspects, a subject in need thereof receives an effective dose of a Tn3 scaffold on Day 169 (±7 days), Day 197 (±7 days), Day 225 (±7 days), Day 253 (±7 days), Day 281 (±7 days), and Day 309 (±7 days).

In aspects, a subject in need thereof is administered an effective dose of a Tn3 scaffold once every 2-4 weeks. In aspects, a subject in need thereof is administered an effective dose of a Tn3 scaffold once every 2 weeks, 4 weeks, 6 weeks, 8 weeks, or 12 weeks. In aspects, a subject in need thereof is administered 1500 mg of a Tn3 scaffold once every 2 weeks for at least 3 doses, once every 4 weeks for at least 12 doses, once every 4 weeks for at least 13 doses, or a combination thereof. In aspects, a subject in need thereof is administered 3000 mg of a Tn3 scaffold once every 2 weeks for at least 3 doses, once every 4 weeks for at least 4 doses, once every 4 weeks for at least 5 doses, or a combination thereof. In aspects, 3000 mg of a Tn3 scaffold is administered every 3 months. In aspects, 3000 mg of a Tn3 scaffold is administered every 12 weeks.

In aspects, a Tn3 scaffold is administered at a dose of about 1500 mg once about every 2 weeks for at least 2 doses and is administered about once a month thereafter. In aspects, a Tn3 scaffold is administered at a dose of about 1500 mg once about every 2 weeks for at least 3 doses and is administered about once a month thereafter. In aspects, a Tn3 scaffold is administered at a dose of about 1500 mg once about every 2 weeks for at least 3 doses and is administered every 4 weeks thereafter. In aspects, a Tn3 scaffold is administered at a dose of about 1500 mg once about every month, once about every two months, or once about every three months. In aspects, a Tn3 scaffold is administered at a dose of about 3000 mg once about every 2 weeks for at least about 2 doses and is administered about once a month, every two months, or every three months thereafter, or combinations thereof. In aspects, a Tn3 scaffold is administered at a dose of about 3000 mg once about every 4 weeks for at least about 2 doses and is administered about once a month, every two months, or every three months thereafter, or combinations thereof. In aspects, a Tn3 scaffold is administered at a dose of about 3000 mg once about every 4 weeks for at least about 2 doses and is administered about every twelve weeks thereafter.

In aspects, a Tn3 scaffold is administered at a dose of about 3000 mg once about every month, once about every two months, or once about every three months. In aspects, a Tn3 scaffold is administered for two or more doses. In aspects, a subject is administered 1500 mg of a Tn3 scaffold of the disclosure every 2 weeks. In aspects, a subject is administered 1500 mg of a Tn3 scaffold of the disclosure every 4 weeks. In aspects, a subject is administered 3000 mg of a Tn3 scaffold of the disclosure every 12 weeks.

In aspects, a method of the disclosure comprises administration of a Tn3 scaffold at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold a dose of 1500 mg of a Tn3 scaffold every 4 weeks. In aspects, a method of the disclosure comprises administration of a Tn3 scaffold comprising SEQ ID NO: 1 at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold comprising SEQ ID NO: 1 at a dose of 1500 mg of a Tn3 scaffold every 2 weeks. In aspects, a method of the disclosure comprises administration of a Tn3 scaffold comprising SEQ ID NO: 1 at a dose of 1500 mg of a Tn3 scaffold every 4 weeks. In aspects, a method of the disclosure comprises administration of a Tn3 scaffold comprising SEQ ID NO: 1 at a dose of 1500 mg of a Tn3 scaffold every two weeks for 3 administrations and then every 4 weeks thereafter. In aspects, a method of the disclosure comprises administration of a Tn3 scaffold comprising SEQ ID NO: 1 at a dose of 3000 mg of a Tn3 scaffold every two weeks for 3 administrations and then every 12 weeks thereafter.

In aspects, a method of the disclosure comprises administration of a Tn3 scaffold at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold at a dose of 1500 mg of a Tn3 scaffold every 4 weeks. In aspects, a method of the disclosure comprises administration of a Tn3 scaffold comprising SEQ ID NO: 22 or SEQ ID NO: 25 at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold comprising SEQ ID NO: 22 or SEQ ID NO: 25 at a dose of 1500 mg of a Tn3 scaffold every 2 weeks. In aspects, a method of the disclosure comprises administration of a Tn3 scaffold comprising SEQ ID NO: 22 or SEQ ID NO: 25 at a dose of 1500 mg of a Tn3 scaffold every 4 weeks. In aspects, a method of the disclosure comprises administration of a Tn3 scaffold comprising SEQ ID NO: 22 or SEQ ID NO: 25 at a dose of 1500 mg of a Tn3 scaffold every two weeks for 3 administrations and then every 4 weeks thereafter. In aspects, a method of the disclosure comprises administration of a Tn3 scaffold comprising SEQ ID NO: 22 or SEQ ID NO: 25 at a dose of 3000 mg of a Tn3 scaffold every two weeks for 3 administrations and then every 12 weeks thereafter.

In aspects, a method of the disclosure comprises administration of a Tn3 scaffold to a subject in need thereof at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold to a subject in need thereof at a dose of 1500 mg of a Tn3 scaffold every 4 weeks. In aspects, a method of the disclosure comprises administration of a Tn3 scaffold comprising SEQ ID NO: 1 to a subject in need thereof at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold comprising SEQ ID NO: 1 to a subject in need thereof at a dose of 1500 mg of a Tn3 scaffold every 2 weeks. In aspects, a method of the disclosure comprises administration of a Tn3 scaffold comprising SEQ ID NO: 1 to a subject in need thereof at a dose of 1500 mg of a Tn3 scaffold every 4 weeks. In aspects, a method of the disclosure comprises administration of a Tn3 scaffold comprising SEQ ID NO: 1 to a subject in need thereof at a dose of 1500 mg of a Tn3 scaffold every two weeks for 3 administrations and then every 4 weeks thereafter. In aspects, a method of the disclosure comprises administration of a Tn3 scaffold comprising SEQ ID NO: 1 to a subject in need thereof at a dose of 1500 mg of a Tn3 scaffold every 2 weeks for 7 administrations. In aspects, a method of the disclosure comprises administration of a Tn3 scaffold comprising SEQ ID NO: 1 to a subject in need thereof at a dose of 1500 mg of a Tn3 scaffold every 4 weeks for 5 administrations. In aspects, a method of the disclosure comprises administration of a Tn3 scaffold comprising SEQ ID NO: 1 to a subject in need thereof at a dose of 3000 mg of a Tn3 scaffold every two weeks for 3 administrations and then every 12 weeks thereafter.

In aspects, a method of the disclosure comprises administration of a Tn3 scaffold to a subject in need thereof at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold to a subject in need thereof at a dose of 1500 mg of a Tn3 scaffold every 2 weeks. In aspects, a method of the disclosure comprises administration of a Tn3 scaffold to a subject in need thereof at a dose of 1500 mg of a Tn3 scaffold every 4 weeks. In aspects, a method of the disclosure comprises administration of a Tn3 scaffold comprising SEQ ID NO: 22 or SEQ ID NO: 25 to a subject in need thereof at a dose of 3000 mg of a Tn3 scaffold every 12 weeks, with a dose of 3000 mg of a Tn3 scaffold at week 0 and week 4. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every 2 weeks. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every 4 weeks. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every 2 weeks for 7 administrations. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every 4 weeks for 5 administrations. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every two weeks for 3 administrations and then every 4 weeks thereafter. In aspects, a method of the disclosure comprises administration of a Tn3 scaffold comprising SEQ ID NO: 22 or SEQ ID NO: 25 to a subject in need thereof at a dose of 3000 mg of a Tn3 scaffold every two weeks for 3 administrations and then every 12 weeks thereafter.

In aspects, a Tn3 scaffold is administered at a dose of about 1500 mg. In aspects, a Tn3 scaffold is administered at a dose of about 3000 mg. In aspects, a Tn3 scaffold is administered at a dose of about 1500 mg every 2 weeks. In aspects, a Tn3 scaffold is administered at a dose of about 1500 mg every 4 weeks. In aspects, a Tn3 scaffold is administered at a dose of about 1500 mg once every 2 weeks for at least 2 or more doses. In aspects, a Tn3 scaffold is administered at a dose of about 1500 mg twice every 2 weeks for at least 2 or more doses. In aspects, a Tn3 scaffold is administered at a dose of about 1500 mg once every 4 weeks for at least 2 or more doses. In aspects, a Tn3 scaffold is administered at a dose of about 1500 mg twice every 4 weeks for at least 2 or more doses. In aspects, a Tn3 scaffold is administered at a dose of about 1500 mg twice every 4 weeks thereafter. In aspects, a Tn3 scaffold is administered at a dose of about 1500 mg every 2 weeks for 3 doses and then every 4 weeks thereafter. In aspects, a Tn3 scaffold is administered at a dose of about 1500 mg every 2 weeks for 2 doses and then every 4 weeks thereafter. In aspects, a Tn3 scaffold is administered at a dose of about 3000 mg every 2 weeks. In aspects, a Tn3 scaffold is administered at a dose of about 3000 mg every 4 weeks. In aspects, a Tn3 scaffold is administered at a dose of about 3000 mg twice every 2 weeks for at least 2 or more doses. In aspects, a Tn3 scaffold is administered at a dose of about 3000 mg twice every 4 weeks for at least 2 or more doses. In aspects, a Tn3 scaffold is administered at a dose of about 3000 mg twice every 12 weeks for at least 2 or more doses. In aspects, a Tn3 scaffold is administered at a dose of about 3000 mg twice every 12 weeks thereafter. In aspects, a Tn3 scaffold is administered at a dose of about 3000 mg every 2 weeks for 3 doses and then every 12 weeks thereafter. In aspects, a Tn3 scaffold is administered at a dose of about 3000 mg every 4 weeks for at least 2 doses, and then every 12 weeks thereafter.

### Methods

In aspects herein, methods are directed to treat, reduce, or eliminate an autoimmune disease or disorder. In aspects, a method comprises administering a Tn3 scaffold of the disclosure. In aspects, a Tn3 scaffold is used to treat SS. In aspects, a Tn3 scaffold is administered to a subject in need thereof to treat SS using any of the dosing schedules disclosed herein. In aspects, a Tn3 scaffold is administered at a dose of about 1500 mg once about every 2 weeks for at least 2 doses and is administered about once a month thereafter. In aspects, a Tn3 scaffold is administered at a dose of about 1500 mg once about every 2 weeks for at least 3 doses and is administered about once a month thereafter, or once every 4 weeks. In aspects, a Tn3 scaffold is administered at a dose of about 1500 once about every month, once about every two months, or once about every three months. In aspects, a Tn3 scaffold is administered at a dose of about 3000 mg once about every month, once about every two months, or once about every three months. In aspects, a Tn3 scaffold is administered at a dose of about 3000 mg one about every 2 weeks for at least 3 doses, and is administered about once every 3 months thereafter. In aspects, a Tn3 scaffold is administered at a dose of about 3000 mg one about every 4 weeks for at least 2 doses, and is administered about once every 3 months thereafter. In aspects, a Tn3 scaffold is administered at a dose of about 3000 mg one about every 4 weeks for at least 2 doses, and is administered about once 12 weeks thereafter. In aspects, a Tn3 scaffold is administered at a dose of about 3000 mg followed by additional administrations at 4 weeks and 12 weeks, and is administered about once every 3 months, or once every 12 weeks, thereafter. In aspects, a Tn3 scaffold is administered for two or more doses.

In aspects, a method comprises treating a subject with SS. In aspects, a method comprises treating a subject with SS with a European Alliance of Associations for Rheumatology (EULAR) Sjögren's Syndrome Disease Activity Index (ESSDAI) of about 1, about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100, about 110, or about 120, or greater. In aspects, a method comprises treating a subject with SS with a EULAR Sjögren's Syndrome Patient Reported Index (ESSPRI) score of about 1, about 2, about 4, about 5, about 6, about 7, about 8, about 9, or about 10.

In aspects, a method comprises treating a subject in need thereof. In aspects, a method comprises treating a subject with SS. In aspects, a method comprises treating a subject with SS with low systemic disease activity. In aspects, a method comprises treating a subject with SS with low systemic disease activity, but moderate to severe symptom state. In aspects, a method comprises treating a subject with SS with moderate systemic disease activity. In aspects, a method comprises treating a subject with SS with severe systemic disease activity. In aspects, a method comprises treating a subject with SS with moderate to severe systemic disease activity. In aspects, a method comprises treating a subject with SS with moderate to severe systemic disease activity by administering a Tn3 scaffold in any dose at in any schedule disclosed herein. In aspects, a method comprises treating a subject with SS and coexisting rheumatoid arthritis (RA). In aspects, a method comprises treating a subject with SS and coexisting systemic lupus erythematosus (SLE). In aspects, moderate to severe systemic disease activity may be defined by EULAR Sjögren's Syndrome Patient Reported Index (ESSPRI)≥5. In aspects, moderate to severe systemic disease activity may be defined by EULAR Sjögren's Syndrome Disease Activity Index (ESSDAI)≥5. In aspects, low systemic disease activity may be defined by ESSDAI<5. In aspects, a method comprises treating a subject with SS with low subjective symptoms by administering a Tn3 scaffold. In aspects, a method comprises treating a subject with SS with moderate to severe subjective symptoms by administering a Tn3 scaffold. In aspects, the subject may have moderate to severe symptomatic activity as defined by ESSPRI score≥5 but with low systemic disease activity defined by ESSDAI score<5. In aspects, moderate to severe systemic disease activity may be defined by ESSDAI score≥5. In aspects, a method comprises treating a subject with severe SS. In aspects, a method comprises treating a subject with SS with an ESSDAI over 14. In aspects, a method comprises treating a subject with SS with an ESSDAI≥14. In aspects, a method comprises treating a subject with SS with an ESSDAI from 14 to about 20, from 14 to about 30, from 14 to about 40, from 14 to about 50, from 14 to about 60, from 15 to about 40, from about 20 to about 70, from about 30 to about 80, from about 40 to about 90, from about 50 to about 100, from about 60 to about 110, from about 70 to about 120, or from about 80 to about 123. In aspects, a Tn3 scaffold is administered at a dose of 1500 mg. In aspects, a Tn3 scaffold is administered at a dose of 3000 mg.

In aspects, a method of treating a subject in need thereof comprises administering an effective dose of an Tn3 scaffold. In aspects, an administration is effective in reducing a disease or a disorder as compared to: a) the disease or disorder in an otherwise comparable subject lacking the administration; or b) a baseline measurement of the disease or disorder in the subject in need thereof. In aspects, an administration is effective as assessed by Diary for Assessing Sjögren's Patient Reported Index (DASPRI). In aspects, a DASPRI score is reduced in a subject administered a Tn3 scaffold by at least or at most about: 1, 2, 3, 4, 5, 10, 15, 20, 25, 30, 35, 40, 45, 50, 60, 70, 80, 90, 95, 96, 97, 98, 99, or 100. In aspects, an administration is effective as assessed by Sjögren's Tool for Assessing Response (STAR). In aspects, a STAR score is increased in a subject administered a Tn3 scaffold by at least or at more about: 1, 2, 3, 4, 5, 6, 7, or 8 points. In aspects, an administration is effective in a reducing an ESSPRI score in a subject in need thereof as compared to a baseline level of the subject. In aspects, an ESSPRI score is reduced in a subject administered a Tn3 scaffold by at least about or at most about: 1-fold, 5-fold, 10-fold, 25-fold, 50-fold, 75-fold, 100-fold, 150-fold, or 250-fold as compared to a baseline level of the subject. In aspects, an ESSPRI score is reduced in a subject administered a Tn3 scaffold by at least about or at most about 1, 1.5, 2, 2.5, 3, 3.5, 4, 4.5, 5, 5.5, 6, 6.5, 7, 7.5, 8, 8.5, 9, 9.5, or 10 points or more as compared to a baseline level of a subject. In aspects, an ESSPRI score is reduced in a subject administered a Tn3 scaffold by at least about or at most about 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 45%, 50%, 55%, 60%, 65%, 70%, 75%, 80%, 85%, 90%, 95%, or up to about 100% as compared to a baseline level of a subject. In aspects, an administration is effective in a reducing an ESSDAI score in a subject in need thereof as compared to a baseline level of the subject. In aspects, an ESSDAI score is reduced in a subject administered a Tn3 scaffold by at least about or at most about: 1-fold, 5-fold, 10-fold, 25-fold, 50-fold, 75-fold, 100-fold, 150-fold, or 250-fold as compared to a baseline level of the subject. In aspects, an ESSDAI score is reduced in a subject administered a Tn3 scaffold by at least about or at most about 1, 1.5, 2, 2.5, 3, 3.5, 4, 4.5, 5, 5.5, 6, 7, 8, 9, 10, 11, 12, 13, 14, 15, 20, 25, 30, 35, 40, 45, 50, 55, 60, 65, 70, 80, 90, or 100 points or more as compared to a baseline level of the subject. In aspects, an ESSDAI score is reduced in a subject administered a Tn3 scaffold by at least about or at most about 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 45%, 50%, 55%, 60%, 65%, 70%, 75%, 80%, 85%, 90%, 95%, or up to about 100% as compared to a baseline level of a subject.

In aspects, a Tn3 scaffold of the disclosure has an increased response rate in a domain selected from the group consisting of: constitutional, lymphadenopathy, glandular, articular, cutaneous, pulmonary, renal, muscular, peripheral nervous system, central nervous system, hematological, and biological, as compared to subjects administered control. In aspects, a response rate is increased from at least about: 5%, 10%, 15%, 20%, 30%, 40%, or 50% as compared to subjected administered control. In aspects, a response rate is increased in a subject administered a Tn3 scaffold of the disclosure in a constitutional domain as compared to the response of a subject administered control. In aspects, a response rate is increased in a subject administered a Tn3 scaffold of the disclosure in a lymphadenopathy domain as compared to the response of a subject administered control. In aspects, a response rate is increased in a subject administered a Tn3 scaffold of the disclosure in a glandular domain as compared to the response of a subject administered control. In aspects, a response rate is increased in a subject administered a Tn3 scaffold of the disclosure in an articular domain as compared to the response of a subject administered control. In aspects, a response rate is increased in a subject administered a Tn3 scaffold of the disclosure in a cutaneous domain as compared to the response of a subject administered control.

In aspects, a Tn3 scaffold is administered at a dose of about 1500 mg via intravenous administration. In aspects, a Tn3 scaffold is administered at a dose of about 3000 mg via intravenous administration. In aspects, a Tn3 scaffold is administered at a dose of about 1500 mg once every 2 weeks via intravenous administration. In aspects, a Tn3 scaffold is administered at a dose of about 1500 mg once every 4 weeks via intravenous administration. In aspects, a Tn3 scaffold is administered at a dose of about 1500 mg twice every 4 weeks thereafter via intravenous administration. In aspects, a Tn3 scaffold is administered at a dose of about 1500 mg every 2 weeks for 3 doses and then every 4 weeks thereafter via intravenous administration. In aspects, a Tn3 scaffold is administered at a dose of about 1500 mg at Weeks 0, 2, and 4, and then every 4 weeks thereafter (Q4W). In aspects, a Tn3 scaffold is administered at a dose of about 1500 mg at Weeks 0, 2, and 4, and once every 4 weeks thereafter (Q4W) via intravenous administration. In aspects, a Tn3 scaffold is administered at a dose of about 1500 mg every 2 weeks for 7 doses. In aspects, a Tn3 scaffold is administered at a dose of about 1500 mg every 2 weeks for 7 doses via intravenous administration. In aspects, a Tn3 scaffold is administered at a dose of about 1500 mg every 4 weeks for 5 doses. In aspects, a Tn3 scaffold is administered at a dose of about 1500 mg every 4 weeks for 5 doses via intravenous administration. In aspects, a Tn3 scaffold is administered at a dose of about 3000 mg twice every 12 weeks thereafter doses via intravenous administration. In aspects, a Tn3 scaffold is administered at a dose of about 3000 mg every 2 weeks for 3 doses and then every 12 weeks thereafter via intravenous administration. In aspects, a Tn3 scaffold is administered at a dose of about 3000 mg every 2 weeks for at least 2 and then every 12 weeks thereafter via intravenous administration. In aspects, a Tn3 scaffold is administered at a dose of about 3000 mg every 4 weeks for at least 2 doses and then every 12 weeks thereafter via intravenous administration.

In aspects, a method of the disclosure comprises administration of a Tn3 scaffold to a subject in need thereof at a dose of 3000 mg of a Tn3 scaffold every 12 weeks, with a dose of 3000 mg of a Tn3 scaffold at week 0 and week 4, via intravenous administration. In aspects, a method of the disclosure comprises administration of a Tn3 scaffold to a subject in need thereof at a dose of 1500 mg of a Tn3 scaffold every 4 weeks via intravenous administration, with a dose of 1500 mg of the Tn3 scaffold at week 0 and week 2. In aspects, a method of the disclosure comprises administration of a Tn3 scaffold comprising SEQ ID NO: 22 or SEQ ID NO: 25 to a subject in need thereof at a dose of 3000 mg of a Tn3 scaffold every 12 weeks, with a dose of 3000 mg of a Tn3 scaffold at week 0 and week 4, via intravenous administration. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every 4 weeks via intravenous administration. In aspects, a method of the disclosure comprises administration of a Tn3 scaffold comprising SEQ ID NO: 22 or SEQ ID NO: 25 to a subject in need thereof at a dose of 1500 mg of a Tn3 scaffold every two weeks for 3 administrations and then every 4 weeks thereafter via intravenous administration. In aspects, a method of the disclosure comprises administration of a Tn3 scaffold comprising SEQ ID NO: 22 or SEQ ID NO: 25 to a subject in need thereof at a dose of 3000 mg of a Tn3 scaffold every two weeks for 3 administrations and then every 12 weeks thereafter via intravenous administration.

In aspects, a composition comprising a Tn3 scaffold is formulated for administration. In aspects, a Tn3 scaffold thereof is formulated at a concentration of at least about or at most about: 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, 200 mg/mL, 205 mg/mL, 210 mg/mL, 215 mg/mL, 220 mg/mL, 225 mg/mL, 230 mg/mL, 235 mg/mL, 240 mg/mL, 245 mg/mL, 250 mg/mL, 255 mg/mL, 260 mg/mL, 265 mg/mL, 270 mg/mL, 275 mg/mL, 280 mg/mL, 285 mg/mL, 290 mg/mL, 295 mg/mL, 300 mg/mL, 305 mg/mL, 310 mg/mL, 315 mg/mL, 320 mg/mL, 325 mg/mL, 330 mg/mL, 335 mg/mL, 340 mg/mL, 345 mg/mL, 350 mg/mL, 355 mg/mL, 360 mg/mL, 365 mg/mL, 370 mg/mL, 375 mg/mL, 380 mg/mL, 385 mg/mL, 390 mg/mL, 395 mg/mL, or up to about 400 mg/mL. In aspects, a Tn3 scaffold effective dose may be formulated at a concentration of about 100 mg/mL.

In aspects, administration of a Tn3 scaffold is effective in eliminating disease in a subject in need thereof. In aspects, administration of a Tn3 scaffold is effective in reducing disease in a subject in need thereof. In some cases, administration of a Tn3 scaffold is effective in eliminating or reducing disease in a subject in need thereof for at least about 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, at least about 5 years, or for the lifetime of the subject. In aspects, administration of a Tn3 scaffold is effective in eliminating disease in a subject in need thereof for at least about 1 week, 2 weeks, 3 weeks, 4 weeks, 5 weeks, 6 weeks, 7 weeks, 8 weeks, 9 weeks, 10 weeks, 11 weeks, or at least about 12 weeks. In aspects, administration of a Tn3 scaffold is effective in eliminating or reducing disease in a subject in need thereof for at least about 1 month, 2 months, 3 months, 4 months, 5 months, 6 months, 7 months, 8 months, 9 months, 10 months, 11 months, or at least about 12 months. In aspects, administration of a Tn3 scaffold is effective in eliminating or reducing disease in a subject in need thereof for at least about 1 year, 2 years, 3 years, 4 years, 5 years, 6 years, 7 years, 8 years, 9 years, or at least about 10 years. In aspects, administration of a Tn3 scaffold is effective in eliminating or reducing disease for the lifetime of a subject in need thereof.

In aspects, a subject in need thereof is administered a Tn3 scaffold. In aspects, a subject in need thereof is administered a Tn3 scaffold as a first line therapy. In aspects, a subject has not been administered one or more prior and/or concomitant therapies for the treatment of SS prior to the administration of a Tn3 scaffold of the disclosure. In aspects, a subject in need thereof has been administered one or more prior and/or concomitant therapies for the treatment of SS prior to the administration of a Tn3 scaffold. In aspects, a prior and/or concomitant therapy comprises a medication and/or vaccine (e.g., over-the-counter [OTC] or prescription medicines, recreational drugs, vitamins, and/or herbal supplements). Exemplary prior and/or concomitant therapies are described below.

### Biologic B-Cell-Depleting Therapy

In aspects, a subject was previously treated with a biologic B-cell-depleting therapy (e.g. rituximab, ocrelizumab, inebilizumab, ofatumumab, belimumab, or ianalumab, or combinations thereof). In aspects, a subject was previously treated with the biologic B-cell-depleting therapy more than about 1 month, 2 months, 3 months, 4 months, 5 months, 6 months, 7 months, 8 months, 9 months, 10 months, 11 months, or about 12 months prior to screening. In aspects, a subject was previously treated with the biologic B-cell-depleting therapy more than about 3 months prior to screening. In aspects, a subject was previously treated with the biologic B-cell-depleting therapy more than about 12 months prior to screening. In aspects, a subject was previously treated with belimumab more than about 3 months prior to screening. In aspects, a subject was previously treated with rituximab, ocrelizumab, inebilizumab, ofatumumab, or ianalumab, or combinations thereof, more than about 12 months prior to screening.

### Corticosteroids

In aspects, a subject was previously treated with a corticosteroid. In aspects, a subject is concomitantly treated with a corticosteroid. In aspects, a corticosteroid is a glucocorticoid. In aspects, a subject was previously treated with an injectable corticosteroid (e.g. intra-articular [IA] or intra-muscular [IM]) or an oral dose of prednisone (or equivalent) at >10 mg day. In aspects, a subject was previously treated with an injectable corticosteroid (e.g. intra-articular [IA] or intra-muscular [IM]) or an oral dose (e.g., prednisone or equivalent) at >10 mg day more than 6 weeks prior to screening. In aspects, a subject was previously treated with a systemic corticosteroid for 2 or more weeks more than 6 months prior to screening. In aspects, a subject is concomitantly treated with an oral corticosteroid at a dose of <10 mg/day (e.g., prednisone or equivalent) wherein the dose is stable for at least 2 weeks or more prior to screening. In aspects, a subject is concomitantly treated with an inhaled corticosteroid, intranasal corticosteroid, or topical corticosteroid at a stable dose. In aspects, a subject is not treated with a corticosteroid.

In aspects, a subject that has an ESSDAI score of ≥5 before the administration is treated or was previously treated with a corticosteroid (e.g., <10 mg/day dose). In aspects, the subject is treated with 1500 mg or 3000 mg of a Tn3 scaffold of the disclosure.

In aspects, a subject that has an ESSDAI score≥5, and an ESSPRI score≤5 before the administration is not administered a corticosteroid. In aspects, the subject is treated with 1500 mg or 3000 mg of a Tn3 scaffold of the disclosure.

### Antimalarials

In aspects, a subject is concomitantly treated with an antimalarial (e.g., chloroquine, hydroxychloroquine, quinacrine, and the like). In aspects, a subject was previously treated with an antimalarial. In aspects, a subject is concomitantly treated with an antimalarial and initiated treatment with the antimalarial more than 8 weeks prior to screening. In aspects, a subject is concomitantly treated with an antimalarial and maintains a stable dose of the antimalarial more than 8 weeks prior to screening.

### Immunomodulatory Agent

In aspects, a subject was previously treated with an immunomodulatory agent. In aspects, a subject is concomitantly treated with an immunomodulatory agent. In aspects, an immunomodulatory agent is also a disease modifying antirheumatic drug (DMARD) (e.g., methotrexate, sulfasalazine, leflunomide, hydroxychloroquine, plaquenil, cevimeline, pilocarpine, ciclosporin, cyclosporine, etanercept, baricitinib, tofacitinib, upadacitinib, infliximab, adalimumab, certolizumab, and golimumab, and combinations thereof). In aspects, a subject was previously treated with a DMARD. In aspects, a subject was previously treated with a DMARD 4 weeks or more prior to screening. In aspects, a subject was previously treated with cevimeline, pilocarpine, and/or cyclosporine 2 weeks or more prior to screening. In aspects, a subject is concomitantly treated with cevimeline, pilocarpine, and/or cyclosporine and the dose is stable as of 2 weeks or more prior to screening.

### NSAID

In aspects, a subject was previously treated with a non-steroidal anti-inflammatory drug (NSAID). In aspects, a subject is concomitantly treated with a NSAID. Exemplary NSAIDS may include, but are not limited to, aspirin, ibuprofen, naproxen, and nabumetone. In aspects, a subject is treated with an NSAID and the NSAID has a stable dose at least 2 weeks or more prior to screening. In aspects, a subject is treated with an NSAID and the NSAID is not administered for the first time at least 2 weeks or more prior to screening.

### Cholinergic Agonist

In aspects, a subject was previously treated with a cholinergic agonist. In aspects, a subject is concomitantly treated with a cholinergic agonist. Exemplary cholinergic agonists include, but are not limited to, cevimeline and pilocarpine.

In aspects, one or more prior and/or concomitant therapies are administered more than about 1, about 2, about 3, about 4, about 5, about 6, about 7, about 8, about 9, about 10, about 11, about 12, about 13, about 14, about 15, about 20, about 25, about 30, about 35, about 40, about 45, or about 52 weeks or more prior to the administration of a Tn3 scaffold. In aspects, one or more prior or concomitant therapies are administered more than about 1 week, about 2 weeks, about 3 weeks, about 4 weeks, about 1 months, about 2 months, about 3 months, about 4 months, about 5 months, about 6 months, about 7 months, about 8 months, about 9 months, about 10 months, about 11 months, or about 12 months or more prior to administration of a Tn3 scaffold.

The dose and dosing regimen of a Tn3 scaffold disclosed herein may be such that any therapeutic effect achieved from administration of a Tn3 scaffold to treat any autoimmune disease or disorder, may be considered to be “long-lasting.” A “long-lasting” effect of a Tn3 scaffold in the treatment of an autoimmune disease or disorder is one in which the therapeutic effect achieved by a Tn3 scaffold is maintained (although a Tn3 scaffold is no longer administered) over at least 4 weeks, at least 6 weeks, at least 8 weeks, at least 10 weeks, at least 12 weeks, at least 16 weeks, at least 20 weeks, at least 24, at least 28 weeks, at least 32 weeks, at least 36 weeks, at least 40 weeks, at least 44 weeks, at least 48 weeks, at least 1 year, at least 2 years, or up to about at least 5 years following administration of the last dose of a course of a Tn3 scaffold. In aspects, less frequent dosing of any of the compositions provided herein may be advantageous. Exemplary advantages of less frequent dosing include but are not limited to reduced frequency of side effects associated with an administered composition, reduced treatment-associated toxicity, increased quality of life for treated subjects, and the like.

### Assessments

In aspects, a subject is assessed. In aspects, a subject is assessed as part of a treatment. In aspects, a subject having a confirmed or probable autoimmune disease or disorder (e.g. SS) is assessed as part of a treatment. An assessment can occur at any point before, during, or after administration with a Tn3 scaffold. In aspects, an assessment is performed before an administration initiates. In aspects, an assessment is performed concurrent with an administration. In aspects, an assessment is performed after an administration finishes.

Any of the below-referenced assessments can occur at any time. In aspects, a subject is assessed by the minute, hourly, daily, weekly, monthly, or yearly. In aspects, an assessment is completed twice daily, biweekly, bimonthly, or semiannually. In aspects, an assessment is performed from day −28, −27,-26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, or up to about 393 days ±7 days post-treatment.

In aspects, a treatment of SS may be characterized by a reduction of at least about 5%, about 10%, about 15%, about 20%, about 30%, about 40%, about 50%, about 60%, about 70%, about 80%, about 90%, about 95%, about 96%, about 97%, about 98%, about 99%, or up to about 100% of clinical symptoms of the disease or disorder, or by a reduction in inflammation, or by a reduction in biomarkers of the disease or disorder, relative to their levels prior to the treatment with a Tn3 scaffold. A reduction of any of these symptoms, or inflammation, or biomarkers, may be a reduction in the symptoms, or inflammation or biomarkers of at least about 10%, 15%, 20%, 25%, about 30%, about 40%, about 50%, about 60%, about 70%, about 75%, about 80%, about 85%, about 90%, about 95%, about 96%, about 97%, about 98%, about 99%, or up to about 100% relative to their levels prior to the initiation of treatment with a Tn3 scaffold. A reduction may be such that the SS is characterized as being in remission.

In aspects, a subject is assessed, and a baseline measurement of disease is determined. In aspects, a subject is assessed, and a baseline measurement of disease is determined at any point before, during, or after administration with a Tn3 scaffold. In aspects, an assessment is performed, and a baseline measurement of disease is determined before an administration with a Tn3 scaffold. In aspects, an assessment is performed, and a baseline measurement of disease is determined concurrent with an administration of a Tn3 scaffold. In aspects, an assessment is performed, and a baseline measurement of disease is determined after an administration of a Tn3 scaffold.

In aspects, a baseline measurement of disease increases or decreases in response to an administration of the disclosure or a lack thereof. In aspects, a baseline measurement of disease may increase and indicate effective treatment of a disease or disorder. In aspects, a baseline measurement of disease may decrease and indicate effective treatment of a disease or disorder. In aspects, a baseline measurement of disease increases or decreases after administration with a Tn3 scaffold. In aspects, a baseline measurement of disease may increase after administration with a Tn3 scaffold and indicate effective treatment of a disease or disorder. In aspects, a baseline measurement of disease may decrease after administration with a Tn3 scaffold and indicate effective treatment of a disease or disorder.

In aspects, a baseline measurement of disease is compared to the measurement of disease of the subject after administration of a Tn3 scaffold. In aspects, a measurement of disease of a subject administered a Tn3 scaffold increases as compared to a baseline measurement of disease measured prior to administration with a Tn3 scaffold. In aspects, a measurement of disease of a subject administered a Tn3 scaffold decreases as compared to a baseline measurement of disease measured prior to administration with a Tn3 scaffold. In aspects, a measurement of disease of a subject administered a Tn3 scaffold increases as compared to a baseline measurement of disease measured prior to administration with a Tn3 scaffold and indicates effective treatment. In aspects, a measurement of disease of a subject administered a Tn3 scaffold decreases as compared to a baseline measurement of disease measured prior to administration with a Tn3 scaffold and indicates effective treatment.

In aspects, an assessment comprises determining treatment efficacy. Efficacy can be determined by any of the assessments of the disclosure. In aspects, effectiveness at preventing, reducing, or eliminating an autoimmune disease or disorder (e.g., SS) is determined by detecting a change in a European Alliance of Associations for Rheumatology (EULAR) Sjögren's Syndrome Disease Activity Index (ESSDAI) score. In aspects, the ESSDAI is reduced by about 1 point, about 2 points, about 3 points, about 4 points, about 5 points, about 6 points, about 7 points, about 8 points, about 9 points, about 10 points, or more from baseline. In aspects, effectiveness at preventing, reducing, or eliminating an autoimmune disease or disorder (e.g., SS) is determined by detecting a change in a EULAR Sjögren's Syndrome Patient Reported Index (ESSPRI) score. In aspects, the ESSPRI is reduced by about 1 point (ESSPRI [1]), about 1.5 points (ESSPRI [1.5]), about 2 points (ESSPRI [2]), about 3 points (ESSPRI [3]), about 4 points (ESSPRI [4]), about 5 points (ESSPRI [5]), about 6 points (ESSPRI [6]), about 7 points (ESSPRI [7]), about 8 points (ESSPRI [8]), about 9 points (ESSPRI [9]), about 10 points (ESSPRI [10]), or more from baseline. In aspects, effectiveness at preventing, reducing, or eliminating an autoimmune disease or disorder is determined evaluating: pharmacokinetic parameters of a Tn3 scaffold used to a treat a subject in need thereof, a change in baseline in a subject's blood level of IgM, rheumatoid factor (RF), sCD40L, CXCL13, a presence of anti-drug antibodies (ADA), and combinations thereof. In aspects, effectiveness at preventing, reducing, or eliminating an autoimmune disease or disorder is determined by evaluating a subject's level of plasma immunoglobulins (IgM, IgG, and IgA), beta-2 microglobulin, high-sensitivity CRP, serum C3, C4, free light chains, cryoglobulins, and serum for anti-SSA (e.g., anti-Ro), anti-SSB (e.g., anti-La), and IgG. In aspects, effectiveness at preventing, reducing, or eliminating an autoimmune disease or disorder is determined by evaluating rheumatoid factor(s) (RF). RF are antibodies with various isotypes and affinities, directed against the Fc portion of immunoglobulin G. The most common rheumatoid factor is an IgM RF, although other immunoglobulin types, including IgG and IgA, can be found.

In aspects, effectiveness at preventing, reducing, or eliminating an autoimmune disease or disorder is determined by evaluating a subject's population of leukocytes, e.g. B lymphocytes. Subject B cells may be present in the peripheral blood. In aspects, subject B cells are present in the salivary glands. In aspects, a subject of the disclosure exhibits disturbed B cell homeostasis comprising diminished frequencies and/or absolute numbers of peripheral CD27+ memory B cells, in particular reduction in the circulating CD27+ IgM+subpopulation. In aspects, a subject of the disclosure comprises an increase in the number of naïve un-switched peripheral memory B cells (CD19+, CD27−, IgD+) with a decrease in the number of the peripheral memory B cells (CD19+, CD27+, IgD−). In aspects, a subject of the disclosure comprises increased frequency of transitional B cells and mature naïve B cells expressing polyreactive antibodies in peripheral blood. Compositions and methods herein are effective at resolving the disturbances of any B cell levels described herein. For example, a subject treated with a composition of the disclosure may exhibit increased frequencies and/or absolute numbers of peripheral CD27+ memory B cells following administration of a composition of the disclosure (e.g., a Tn3 scaffold). In aspects, a subject administered a composition of the disclosure exhibits an increase in the CD27+ IgM+subpopulation as compared to a baseline level of the subject. In aspects, a subject of the disclosure exhibits a decrease in the number of naïve un-switched peripheral memory B cells (CD19+, CD27−, IgD+) with an increase in the number of the peripheral memory B cells (CD19+, CD27+, IgD−) as compared to baseline.

In aspects, effectiveness at preventing, reducing, or eliminating an autoimmune disease or disorder is determined by evaluating a subject's population of CD27+ memory B cells, marginal zone B cells, plasmablasts, plasma cells, and combinations thereof. In aspects, effectiveness at preventing, reducing, or eliminating an autoimmune disease or disorder is determined by evaluating a subject's B cell population, for example, Ki67+ post-switch memory B cells (of CD27+/IgD−/CD19+ cells), Ki67+/CD27+ memory B cells, CD27br/CD38br/IgD-plasmablasts, CD11chigh memory B cells or CD11cbr atypical memory cells, or combinations thereof. In aspects, effectiveness at preventing, reducing, or eliminating an autoimmune disease or disorder is determined by evaluating a subject's level of TfH cells. In aspects, a subject's level of TfH cells are evaluated by evaluating the percent positive CXCR5+ and/or ICOS+ cells. In aspects, a subject's level of TfH cells are evaluated by evaluating the percent positive CXCR5+ and/or ICOS+ cells of identified CD4+ and/or CD3+ cells.

In aspects, a subject of the disclosure exhibits a disturbance of B cells within an exocrine gland infiltrate. For example a disturbance may comprise one or more of: a presence of germinal center-like structures, increased frequency of IgG plasma cells as compared to a healthy subject, autoreactive B cells and/or plasma cells, increased levels of B cell-associated cytokines and chemokines (e.g., IL-6, IL-21, BAFF, APRIL, CXCL12, CXCL13) as compared to a healthy subject, presence of clonal B cell populations and/or autoantibody production plasma cells, and any combination thereof. In aspects, a subject administered a composition of the disclosure exhibits reduced levels of a B cell-associated cytokine or chemokine selected from the group consisting of: IL-6, IL-21, BAFF, APRIL, CXCL12, CXCL13, and any combination thereof. In aspects, a reduction is of at least about or at most about: 5%, 10%, 20%, 40%, 60%, 80%, 100%, or 150%. In aspects, evaluating the level of one or more parameters (e.g., B cells) comprises quantifying the number of said parameter(s) in a sample of a subject.

### Assessment of Inclusion

In aspects, a subject in need thereof is assessed prior to beginning treatment.

In aspects, a subject in need thereof may be either male or female. In aspects, a subject in need thereof is an adult subject. In aspects, a subject in need thereof may be aged at least about 18 years of age. In aspects, a subject is from 18-100 years of age.

In aspects, a subject in need thereof has a diagnosis of SS. In aspects, a subject in need thereof has a diagnosis of SS according to American College of Rheumatology (ACR) criteria. In aspects, a subject in need thereof has a diagnosis of SS according to 2016 American College of Rheumatology (ACR) criteria. In aspects, a subject in need thereof has an ESSPRI score of ≥5. In aspects, a subject in need thereof has an ESSDAI score of <5. In aspects, a subject in need thereof has an ESSDAI score of ≥5. In aspects, a subject in need thereof is positive for anti-SSA (e.g., anti-Ro) autoantibodies. In aspects, a subject in need thereof is positive for rheumatoid factor (RF). In aspects, a subject in need thereof is positive for both anti-SSA (e.g., anti-Ro) autoantibodies and RF. In aspects, a subject in need thereof has a diagnosis of rheumatoid arthritis (RA). In aspects, a subject in need thereof has a diagnosis of systemic lupus erythematosus (SLE). In aspects, a subject in need thereof has SS and coexisting RA. In aspects, a subject in need thereof has SS and coexisting SLE. An assessment of inclusion of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, up to about 0 days post treatment initiation.

### ESSPRI

In aspects, an assessment can comprise an ESSPRI evaluation. In aspects, an ESSPRI evaluation is a self-evaluation (Seror et al, 2011). In aspects, an ESSPRI evaluation uses a 0 to 10 numerical analog scale (ranging from 0 [no symptoms] to 10 [maximal imaginable severity]), one for the assessment of each of the 3 domains: dryness, fatigue, and pain (articular and/or muscular). ESSPRI fatigue has been shown to be a main predictor of poor quality of life (QoL) in a subject with SS, along with pain. In aspects, the ESSPRI dryness domain has been shown to correlate with quality of sleep, presence and degree of anxiety and depression (Gandia et al., 2014), and overall QOL (Schmalz et al., 2020). In aspects, a subject in need thereof has an ESSPRI score of ≥5, which may be considered as the cut-off point for “unsatisfactory symptom state” (Seror et al., 2016).

The weights of the domains may be identical and the mean of the scores of the 3 domains represents the final score. The recall period may be stated in each question as “the last 2 weeks”. In aspects, a subject has an ESSPRI score from about 1 to about 100 before administration of a composition of the disclosure. In aspects, a subject has an ESSPRI score from about 1-10, 5-15, 10-20, 15-25, 20-30, 25-35, 30-40, 35-45, 40-50, 45-55, 50-60, 55-65, 60-70, 65-75, 70-80, 75-85, 80-90, 85-95, 90-100, or about 95-100 before administration of a composition of the disclosure. In aspects, a subject has an ESSPRI score from about 5-6, 5-7, 6-8, 6-9, or about 5-10 before administration of a composition of the disclosure.

The ESSPRI [1.5] refers to an at least 1.5-point reduction from baseline in an ESSPRI score.

In aspects, treatment with a composition of the disclosure is effective in reducing an ESSPRI score. In aspects, an ESSPRI score is reduced by at least about: 0.6, 0.7, 0.8, 0.9 1, 1.1, 1.3, 1.4, 1.5, 1.6, 1.7, 1.8, 1.9, 2, 3, 4, 5, 6, 7, 8, 9, or 10 points. In aspects, an ESSPRI score is reduced by up to about 0.6, 0.7, 0.8, 0.9, 1, 1.1, 1.3, 1.4, 1.5, 1.6, 1.7, 1.8, 1.9, 2, 3, 4, 5, 6, 7, 8, 9, or 10 points. In aspects, an ESSPRI score is reduced from about 0.6-5, 0.6-10, 1-10, 1-5, 2-10, or 5-10 points. In aspects, an ESSPRI score is reduced as compared to an otherwise comparable method wherein the subject of the otherwise comparable method is not administered a Tn3 scaffold, for example, the reduction may be about: 10%, 15%, 20%, 25%, 50%, 60%, 70%, 80%, 90%, 95%, 96%, 97%, 98%, 99%, or up to 100% as compared to an ESSPRI score in an otherwise comparable method wherein the subject of the otherwise comparable method is not administered a Tn3 scaffold. In aspects, an ESSPRI score is reduced as compared to an otherwise comparable method wherein the subject of the otherwise comparable method is not administered a Tn3 scaffold, for example, the reduction may be about: 10%, 15%, 20%, 25%, 50%, 60%, 70%, 80%, 90%, 95%, 96%, 97%, 98%, 99%, or up to 100% as compared to baseline.

In aspects, the ESSPRI score is assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, the ESSPRI score is assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. As described herein, an ESSPRI score can be assessed at any time before, during, or after treatment with a Tn3 scaffold of the disclosure.

Patient-Reported Outcomes Measurement Information System (PROMIS) Fatigue Short Form 10a survey

In aspects, an assessment comprises a PROMIS fatigue 10a survey. A PROMIS fatigue 10a survey may assess a range of self-reported symptoms, from mild subjective feelings of tiredness to an overwhelming, debilitating, and sustained sense of exhaustion. In aspects, fatigue is divided into the experience of fatigue (frequency, duration, and intensity) and the impact of fatigue on physical, mental, and social activities. In aspects, a PROMIS fatigue 10a survey assesses symptoms over the past week. In aspects, a PROMIS fatigue 10a survey may comprise assessing a subject in need thereof administered a Tn3 scaffold of the disclosure as compared to baseline. In aspects, a subject's self-reported symptoms are improved as compared to an otherwise comparable method wherein the subject of the otherwise comparable method is not administered Tn3 scaffold of the disclosure.

In aspects, a PROMIS fatigue 10a survey to Tn3 scaffold of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, PROMIS fatigue is assessed at any time before, during, or after treatment with a Tn3 scaffold of the disclosure.

5-level EQ-ED (EQ-5D-5L)

In aspects, an assessment can comprise a EQ-5D-5L survey. The EQ-5D-5L survey provided to a subject to capture information covering five dimension of their health state: mobility, self-care, usual activities, pain/discomfort, and anxiety/depression. The EQ-5D-5L survey has five response levels: no problems (1), slight problems (2), moderate problems (3), severe problems (4), unable to/extreme problems. The subject indicates their health by choosing the most appropriate response level for each of the five dimensions. In aspects, a EQ-5D-5L survey assesses symptoms over the past week. In aspects, a EQ-5D-5L survey may comprise assessing a subject in need thereof administered a Tn3 scaffold of the disclosure as compared to baseline. In aspects, a subject's self-reported symptoms are improved as compared to an otherwise comparable method wherein the subject of the otherwise comparable method is not administered Tn3 scaffold of the disclosure.

In aspects, a EQ-5D-5L survey to Tn3 scaffold of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, EQ-5D-5L is assessed at any time before, during, or after treatment with a Tn3 scaffold of the disclosure.

36-item Short Form Survey (SF-36)

In aspects, an assessment can comprise a SF-36. The SF-36 is a 36-item general health status assessment that may capture information about 8 health domains: Physical Functioning, Role Physical, Bodily Pain, General Health, Vitality, Social Functioning, Role Emotional, and Mental Health. The SF-36 provides scores for each domain as well as 2 psychometrically based summary scores: Physical Component Score (PCS) and Mental Component Score (MCS). The recall period for the acute version is one week (i.e., “last week”). The SF-36 is a widely used, validated professional quality of life score that encompasses multiple domains and is sensitive to change (Hemingway et al, 1997). The SF-36 PCS is derived from multiple physical domains and has been shown to be validated and responsive in related autoimmune diseases (Kosinski et al, 1999; Devilliers et al, 2015). The SF-36 PCS score can comprehensively capture improvements in physical activity and mental functioning, as these scores are based on the assessment of several symptoms proximal to subjects with SS such as pain and mental and physical health (Sjögren's Foundation 2021).

In aspects, a SF-36 survey may comprise assessing a subject in need thereof administered a Tn3 scaffold compared to baseline. In aspects, a subject's functional health and wellbeing is improved as compared to an otherwise comparable method wherein the subject of the otherwise comparable method is not administered a Tn3 scaffold.

In aspects, an SF-36 to a Tn3 scaffold of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a SF-36 to a Tn3 scaffold of the disclosure can be assessed on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, SF-36 is assessed at any time before, during, or after treatment with a Tn3 scaffold of the disclosure.

### Location of Dryness Improvement Items

Assessments of the disclosure can comprise location of dryness improvement items. The Location of Dryness Improvement Items is a series of questions assessing location of dryness. In aspects, questions are completed during screening and asks subjects to rank locations of dryness most important to them to improve (1=most important location to improve, 4=least important location to improve). Follow-on questions comprise asking subjects to pick the location that has improved the most (if any) at 2 different time points: Week 24 (Question 2) and Week 48 (Question 3). Additional timepoints are contemplated herein including 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, location of dryness improvement is assessed at any time before, during, or after treatment with a Tn3 scaffold of the disclosure.

In aspects, treatment with a composition of the disclosure is effective in reducing dryness as determined by the location of dryness improvement assay. In aspects, a subject treated with a composition of the disclosure experiences reduced dryness at one or more locations with identified dryness prior to treatment. Dryness can be self-identified or medically identified by a professional. In aspects, reduced dryness is determined by self-identification and comprises a reduction in self-identification of at least about or at most about 1 event, 2 events, 3 events, 4 events or 5 events.

### Functional Assessment of Chronic Illness Therapy (FACIT)-Fatigue

In aspects, an assessment can comprise a FACIT-Fatigue evaluation. The FACIT-Fatigue is a subject-completed, 13-item questionnaire used to assess the impact of fatigue. The FACIT-Fatigue recall period is 7 days. Responses range from 0 (Not at all) to 4 (Very Much). In aspects, a FACIT-Fatigue score for a question is from about 0, 1, 2, 3, or up to about 4. In aspects, a FACIT-Fatigue score for a question is from about 0 to about 4 or about 1 to about 4. To calculate the total score, the negatively stated items are reversed by subtracting the response from “4”. Final scores are the sum of the responses and range from 0 to 52. In aspects, a FACIT-Fatigue total score is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or up to about 52. Higher scores indicate better quality of life. In aspects, an FACIT-Fatigue evaluation may comprise assessing a subject in need thereof administered a Tn3 scaffold as compared to baseline. In aspects, a subject's FACIT-Fatigue score is increased as compared to an otherwise comparable method wherein the subject of the otherwise comparable method is not administered a Tn3 scaffold.

In aspects, treatment with a composition of the disclosure is effective in reducing a FACIT-Fatigue score. In aspects, a FACIT-Fatigue score is reduced by at least about: 1, 2, 3, 4, 5, 6, 7, 8, 9, 10, 15, 20, 25, 30, 35, 40, 45, 50, or 52 points. In aspects, a FACIT-Fatigue score is reduced by up to about 1, 2, 3, 4, 5, 6, 7, 8, 9, 10, 15, 20, 25, 30, 35, 40, 45, 50, or 52 points. In aspects, a FACIT-Fatigue score is reduced from about 1-5, 2-10, 5-10, 5-20, 10-20, 25-45, or 20-30 points.

In aspects, a FACIT-Fatigue evaluation to a Tn3 scaffold of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a FACIT-fatigue evaluation to a Tn3 scaffold of the disclosure can be assessed on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, FACIT-Fatigue is assessed at any time before, during, or after treatment with a Tn3 scaffold of the disclosure.

Visual Analog Scale (VAS) of Ocular, Oral, and/or Vaginal Dryness

In aspects, an assessment can comprise a VAS of ocular, oral, and/or vaginal dryness. The VAS Oral/Ocular/Vaginal consists of 3 instruments that use a continuous 100 mm VAS (0 mm=best, 100 mm=worst) to assess change in the severity of oral, ocular, and vaginal dryness throughout the study. The subject is asked to place a line perpendicular to the VAS line at the point that represents the symptom intensity over the past 2 weeks. The VAS oral rates the question “How dry your mouth feels most of the time” (not dry at all 0 mm; dry as a desert 100 mm). The VAS ocular rates the question “How dry do your eyes feel most of the time” (not dry at all 0 mm; very dry 100 mm). The VAS vaginal ask respondents (as appropriate) to rate symptoms of vaginal dryness (no symptoms 0 mm; worst possible symptoms 100 mm). In aspects, a VAS Oral/Ocular/Vaginal survey is scored from about 0 to about 100, about 1 to about 100, about 10 to about 100, or from about 0 to about 90. In aspects, a VAS ocular/oral/vaginal survey is scored from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to up to about 100. In aspects, a VAS Oral/Ocular/Vaginal evaluation may comprise assessing a subject in need thereof administered a Tn3 scaffold as compared to baseline. In aspects, a subject's VAS Oral/Ocular/Vaginal score is decreased as compared to an otherwise comparable method wherein the subject of the otherwise comparable method is not administered a Tn3 scaffold. In aspects, a baseline (VAS) Oral/Ocular/Vaginal score is reduced by about 3%, 5%, 10%, 20%, 30%, 40%, 50%, 60%, 70%, 80%, 90%, or up to about 100% as compared to a subject's VAS Oral/Ocular/Vaginal score prior to administration of a Tn3 scaffold.

In aspects, treatment with a composition of the disclosure is effective in reducing a VAS Oral/Ocular/Vaginal score. In aspects, a VAS Oral/Ocular/Vaginal score is reduced by at least about: 1, 2, 3, 4, 5, 6, 7, 8, 9, 10, 15, 20, 25, 30, 35, 40, 45, 50, or 55 points. In aspects, a VAS Oral/Ocular/Vaginal score is reduced by up to about 1, 2, 3, 4, 5, 6, 7, 8, 9, 10, 15, 20, 25, 30, 35, 40, 45, 50, 55, 60, 65, 70, 75, 80, 85, 90, 95, or up to about 100 points. In aspects, a VAS Oral/Ocular/Vaginal score is reduced from about 1-5, 2-10, 5-10, 5-20, 10-20, 20-30, 30-40, 40-50, 50-60, 60-70, 70-80, 80-90, or about 90-100 points.

In aspects, a VAS Oral/Ocular/Vaginal evaluation to a Tn3 scaffold of the disclosure can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a VAS Oral/Ocular/Vaginal evaluation to a Tn3 scaffold of the disclosure can be assessed on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, VAS Oral/Ocular/Vaginal dryness is assessed at any time before, during, or after treatment with a Tn3 scaffold of the disclosure.

### Patient Global Impression of Severity (PGIS)

In aspects, an assessment can comprise a PGIS survey. The PGIS is a single-item questionnaire designed to capture a subject's perception of the severity of the worst overall SS symptoms (e.g., pain, fatigue, and/or dryness) over the last 7 days on a 5-point categorical response scale (1=none, 2=mild, 3=moderate, 4=severe, or 5=very severe). In aspects, a PGIS score may comprise assessing a subject in need thereof administered a Tn3 scaffold as compared to baseline. In aspects, a subject's PGIS score is decreased as compared to an otherwise comparable method wherein the subject of the otherwise comparable method is not administered a Tn3 scaffold. A reduction can be of from about 1, 2, 3, 4, or up to about 5 points. In aspects, a subject receives 3 symptom-specific PGIS items (e.g., pain, fatigue, and/or dryness) as well as an overall symptom PGIS. An overall symptom PGIS can comprise additional symptoms beyond pain, fatigue, and/or dryness.

In aspects, treatment with a composition of the disclosure is effective in reducing a PGIS score. In aspects, a PGIS score is reduced by at least about: 1, 2, 3, 4, or 5 points. In aspects, a PGIS score is reduced by up to about 1, 2, 3, 4, or 5 points. In aspects, a PGIS score is reduced from about 1-5, 2-5, 1-4, or 3-5 points.

In aspects, the PGIS survey can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, the PGIS survey can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, PGIS is assessed at any time before, during, or after treatment with a Tn3 scaffold of the disclosure.

### Patient Global Impression of Change (PGIC)

In aspects, an assessment can comprise a PGIC survey. The PGIC is a single-item questionnaire designed to capture a subject's perception of change in their SS symptoms (e.g., pain, fatigue, and/or dryness) from starting treatment. Change in severity is captured using a 5-point scale (1-much better, 2=a little better, 3=no change, 4=a little worse, or 5=much worse). In aspects, a PGIC score may comprise assessing a subject in need thereof administered a Tn3 scaffold as compared to baseline. In aspects, a subject's PGIC score is decreased as compared to an otherwise comparable method wherein the subject of the otherwise comparable method is not administered a Tn3 scaffold. A reduction can be of from about 1, 2, 3, 4, or up to about 5 points. In aspects, subjects receive 3 symptom-specific PGIC items (pain, fatigue, and/or dryness), as well as overall symptom PGIC.

In aspects, treatment with a composition of the disclosure is effective in reducing a PGIC score. In aspects, a PGIC score is reduced by at least about: 1, 2, 3, 4, or 5 points. In aspects, a PGIC score is reduced by up to about 1, 2, 3, 4, or 5 points. In aspects, a PGIC score is reduced from about 1-5, 2-5, 1-4, or 3-5 points.

In aspects, the PGIC survey can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, the PGIC survey can be assessed on about Day 15 (−3 days to +1 day), Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, PGIC is assessed at any time before, during, or after treatment with a Tn3 scaffold of the disclosure.

### Unstimulated Salivary Flow

Assessments of the disclosure can comprise an unstimulated salivary flow assay. The unstimulated salivary flow assessment can be performed prior to the stimulated salivary flow collection but other periods are also contemplated. Subjects receiving standard of care for xerostomia at screening may discontinue use of pilocarpine and/or cevimeline for at least about 5 hours, 6 hours, 7 hours, 8 hours, 9 hours, 10 hours, 11 hours, 12 hours, 13 hours, 14 hours, or 15 hours and/or artificial saliva for at least about 1 hour, 2 hours, 3 hours, 4 hours, 5 hours, 6 hours, 7 hours, 8 hours, 9 hours, or 10 hours prior to saliva collection. Participants should refrain from eating or drinking for at least about 30 minutes, 60 minutes, 90 minutes, 120 minutes, or 175 minutes prior to saliva collection.

For the unstimulated salivary flow collection, subjects refrain from swallowing or speaking throughout the collection time, with the exception of a single swallow, immediately prior to the initiation of the timing of the collection. In aspects, the total duration of the procedure is about 1 minutes, 2 minutes, 3 minutes, 4 minutes, 5 minutes, 6 minutes, 7 minutes, 8 minutes, 9 minutes, or 10 minutes during which subjects allow saliva to accumulate in the oral cavity for a period of 60 seconds prior to emptying into a pre-weighed container. This is to be repeated a total of about 1, 2, 3, 4, 5, 6, 7, or 8 times during the test. The weight of the container needs to be recorded prior to the initiation and at the end of the procedure.

In aspects, a subject treated with a composition of the disclosure experiences increased salivary flow as determined by unstimulated salivary flow assay. In aspects, a subject exhibits increased salary flow wherein their container comprises increased weight as compared to the weight of the container at baseline. In aspects, weight is increased by at least about or at most about 5%, 10%, 20%, 30%, 40%, 50%, 75%, 100%, 120%, 150%, or up to about 200%.

### Female Sexual Function Index (FSFI)

In aspects, an assessment can comprise a FSFI survey. The FSFI survey was designed to define female sexual function in clinical and nonclinical samples by establishing clear endpoints and outcomes for female sexual function research (Rosen et al, 2000). The FSFI is 19-item, self-reported, and validated questionnaire assessing function over the past 4 weeks in the following domains: desire, arousal, lubrication, orgasm, satisfaction, and pain. Each question is scored on scale of 0 (no sexual activity) or 1 (maximal dysfunction) to 5 (no sexual dysfunction). The final score is computed in the following manner: to normalize each domain based on item number, the sum of each domain score is first multiplied by a domain factor ratio (0.6 for desire; 0.3 for arousal; 0.3 for lubrication; 0.4 for orgasm; 0.4 for satisfaction; and 0.4 for pain) and then the domain scores are added to produce a final score. In aspects, a FSFI survey is scored from about 0 to about 36, about 1 to about 36, about 2 to about 36, about 0 to about 30, about 1 to about 30, or from about 2 to about 30. In aspects, a FSFI survey is scored from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, to up to about 36. This questionnaire may be completed by female subjects. In aspects, a FSFI score may comprise assessing a subject in need thereof administered a Tn3 scaffold as compared to baseline. In aspects, a subject's FSFI score is increased as compared to an otherwise comparable method wherein the subject of the otherwise comparable method is not administered a Tn3 scaffold.

In aspects, treatment with a composition of the disclosure is effective in increasing a FSFI survey score. In aspects, a FSFI score is increased by at least about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, to up to about 36 points. In aspects, a PGIC score is increased by up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, to up to about 36. In aspects, a FSFI score is increased from about 1-36, 1-30, 1-20, 1-10, 5-10, 15-25, 20-36, or 25-36 points.

In aspects, the FSFI survey can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, the FSFI survey can be assessed on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, FSFI is assessed at any time before, during, or after treatment with a Tn3 scaffold of the disclosure.

### ESSDAI

Assessments of the disclosure can comprise a ESSDAI evaluation. In aspects, a ESSDAI evaluation includes a physical exam. The ESSDAI is a systemic disease activity index that includes organ-by-organ definitions of disease activity (Seror et al, 2010). The ESSDAI grades disease activity in 12 domains (cutaneous, respiratory, renal, articular, muscular, peripheral nervous system, central nervous system, hematological, glandular, constitutional, lymphadenopathic, and biological). The weights of each domain were obtained by multiple regression modeling, using the Physician's Global Assessment of Activity as gold standard.

Each domain is weighted from 1 (Biologic domain) to 6 (Muscular domain) and has 3 or 4 levels of activity per domain, ranging from 0 (no activity) to 3 or 4 (severe activity).

The theoretical range of values for the ESSDAI is 0 to 123, with the final score being calculated as follows: 1) Final score=Sum of all 12 domain scores; 2) Domain score=Activity level×Domain weight.

Low-activity status is defined as ESSDAI<5, moderate-activity as 5≤ESSDAI≤13, and severe activity as ESSDAI≥14 (Seror et al, 2016). The ESSDAI may be an appropriate primary endpoint because it is a validated and widely used measure of systemic disease activity in SS (Seror et al, 2015). It was found to have good construct validity with reliable scoring; in addition, systemic scores demonstrated good sensitivity to change in subjects whose disease activity improves. ESSDAI [3] can refer to a 3 point reduction from baseline in an ESSDAI score of a subject in need thereof previously administered a Tn3 scaffold of the disclosure. ESSDAI [4] can refer to a 4 point reduction from baseline in an ESSDAI score of a subject in need thereof previously administered a Tn3 scaffold of the disclosure. ESSDAI [5] can refer to a 5 point reduction from baseline in an ESSDAI score of a subject in need thereof previously administered a Tn3 scaffold of the disclosure. ESSDAI [6] can refer to a 6 point reduction from baseline in an ESSDAI score of a subject in need thereof previously administered a Tn3 scaffold of the disclosure.

In aspects, an ESSDAI score is from about 0 to 123, about 1 to about 123, about 2 to about 123, about 10 to about 120, or about 5 to about 120. In aspects, an ESSDAI score is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, or up to about 123. In aspects, a subject has an ESSDAI score from about 5-7, 5-10, 5-13, or 10-13 before treatment with a composition of the disclosure.

In aspects, treatment with a composition of the disclosure is effective in reducing an ESSDAI score. In aspects, an ESSDAI score is reduced by at least about: 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, or 120 points. In aspects, an ESSDAI score is reduced by up to about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, or 120 points. In aspects, an ESSDAI score is reduced from about 1-5, 2-10, 5-10, 5-20, 10-20, 20-30, 30-40, 40-50, 50-60, 60-70, 70-80, 80-90, 90-100, 100-110, or 110-120 points.

In aspects, treatment with a composition of the disclosure is effective in reducing an ESSDAI score. In aspects, an ESSDAI score is reduced by at least about or at most about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In aspects, an ESSDAI score is reduced by about 1-10%, 1-20%, 5-20%, 10-30%, 20-30%, 25-30%, 25-35%, 30-40%, 35-55%, 40-60%, 50-70%, 60-80%, 70-90%, 80-100%, or by about 90-100%.

In aspects, the ESSDAI evaluation can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, the ESSDAI evaluation can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, ESSDAI is assessed at any time before, during, or after treatment with a Tn3 scaffold of the disclosure.

### Clinical EULAR Sjögren's Syndrome Disease Activity Index (ClinESSDAI)

In aspects, an assessment can comprise a ClinESSDAI evaluation. The ClinESSDAI is a validated SS disease activity index based on ESSDAI that excludes the biological domain and assigns different weights assigned to each domain. ClinESSDAI was developed to diminish possible associations between the B-cell biomarkers measured by the ESSDAI biological domain and clinical activity measures (Seror et al, 2016). The theoretical range of values for the ClinESSDAI is 0 to 135. Similar to ESSDAI, low-activity status is defined as <5, moderate-activity as 5≤ClinESSDAI≤13, and high-activity as ≥14 (Seror et al, 2016). ClinESSDAI has been validated and shown to correlate well with ESSDAI and is considered a useful tool to detect change independent of biological effect of the drug (Seror et al, 2016; Dumusc et al, 2018; Quartuccio et al, 2017).

In aspects, a ClinESSDAI score is from about 0 to 135, about 1 to about 135, about 2 to about 135, about 10 to about 130, or about 5 to about 130. In aspects, an ClinESSDAI score is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or up to about 135. In aspects, a ClinESSDAI score may comprise assessing a subject in need thereof administered a Tn3 scaffold as compared to baseline. In aspects, a subject's ClinESSDAI score is decreased as compared to an otherwise comparable method wherein the subject of the otherwise comparable method is not administered a Tn3 scaffold.

In aspects, treatment with a composition of the disclosure is effective in reducing a ClinESSDAI score. In aspects, a ClinESSDAI score is reduced by at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135 points. In aspects, a ClinESSDAI score is reduced by up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135 points. In aspects, a ClinESSDAI score is reduced from about 1-5, 2-10, 5-10, 5-20, 10-20, 20-30, 30-40, 40-50, 50-60, 60-70, 70-80, 80-90, 90-100, 100-110, 110-120, 120-130, or 125-135 points.

In aspects, a ClinESSDAI evaluation can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a ClinESSDAI can be made on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, ClinESSDAI is assessed at any time before, during, or after treatment with a Tn3 scaffold of the disclosure.

### 28-Joint Assessment (TJC and SJC)

In aspects, an assessment can comprise a 28-joint assessment. In aspects, a 28-joint assessment comprises a tender joint count (TJC). In aspects, a 28-joint count comprises a swollen-joint count (SJC). In aspects, a 28-joint count comprises a TJC and an SJC. The 28-joint assessment will assess the following joints for tenderness and swelling: left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities, and left and right knee of the lower extremities. Each of the 28 joints may be evaluated for the presence of synovitis. At the start of the 28-joint count (prior to assessment of tenderness and swelling), a subject may be asked if they have experienced or are experiencing pain in any of the 28 joints. In aspects, a 28-joint assessment score is from about 0 to about 28. In aspects, a 28-joint assessment score is from about 0, 1, 2, 3, 4, 5, 6, 7, 8, 9, 10, 11, 12, 13, 14, 15, 16, 17, 18, 19, 20, 21, 22, 23, 24, 25, 26, 27, up to about 28. In aspects, a 28-joint assessment may comprise assessing a subject in need thereof administered a Tn3 scaffold as compared to baseline. In aspects, a subject's 28-joint assessment score is decreased as compared to an otherwise comparable method wherein the subject of the otherwise comparable method is not administered a Tn3 scaffold.

In aspects, treatment with a composition of the disclosure is effective in reducing a 28-joint assessment score. In aspects, a 28-joint assessment score is reduced by at least about: 1, 2, 3, 4, 5, 6, 7, 8, 9, 10, 11, 12, 13, 14, 15, 16, 17, 18, 19, 20, 21, 22, 23, 24, 25, 26, 27, or 28 points. In aspects, a 28-joint assessment score is reduced by up to about 1, 2, 3, 4, 5, 6, 7, 8, 9, 10, 11, 12, 13, 14, 15, 16, 17, 18, 19, 20, 21, 22, 23, 24, 25, 26, 27, or 28 points. In aspects, a 28-joint assessment score is reduced from about 1-28, 1-5, 2-10, 5-10, 1-20, 5-20, 10-15, 10-20, 15-25, or 20-28 points.

In aspects, a 28-joint assessment can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a 28-joint assessment can be made on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, 28 joint count is assessed at any time before, during, or after treatment with a Tn3 scaffold of the disclosure.

### Stimulated Salivary Flow

In aspects, an assessment can comprise a stimulated salivary flow measurement. In aspects, whole stimulated salivary flow will be measured to objectively assess functional changes in the salivary glands during treatment with a Tn3 scaffold of the disclosure. A subject receiving standard of care for xerostomia at screening may discontinue use of pilocarpine or cevimeline for at least 12 hours and artificial saliva for at least 3 hours prior to saliva collection. A subject may be prohibited from eating or drinking for at least 90 minutes prior to saliva collection. Saliva may be collected at the same time across all visits.

For whole stimulated salivary flow rate measurements, an approximately 5×5 cm parafilm square is rolled and given to a subject to be chewed like a gum at a rate of approximately 60 strokes per minute. The subject should be seated upright with eyes open and head tilted slightly forward and start chewing the parafilm for 60 seconds, at which point all the collected saliva should be spit in an extra (not pre-weighed) falcon tube, keeping the parafilm in the mouth. This first collection accustoms the subject to the procedure. Three rounds of salivary collection, each after 20 seconds of chewing, follow in a pre-weighed falcon tube. Those rounds are continuous, but timing should be stopped during the collection of the saliva and restart immediately after the saliva deposition in a pre-weighed falcon tube, for a total stimulation time of 60 seconds. If collection for 60 seconds is not feasible due to the subject inability, the total collection time should be recorded. Total stimulated saliva collected is assessed by subtracting the weight of the tube prior to collection from the final weight of the. In aspects, a change from baseline in stimulated salivary flow is determined.

In aspects, a stimulated salivary flow measurement can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a stimulated salivary flow measurement can be assessed on about Day 1, Day 85 (±7 days), Day 169 (±7 days), Day 253 (±7 days), or on about Day 337 (±7 days) post treatment initiation. In aspects, salivary flow is assessed at any time before, during, or after treatment with a Tn3 scaffold of the disclosure.

### European Quality of Life 5-Dimension 5-Level Version (EQ-5D-5L)

An assessment of the disclosure can be EQ-5D-5L. The 5-domain, 5-level version of the EQ (EQ-5D-5L) is a generic PRO instrument that measures health status. It consists of a descriptive system and the EQ visual analogue scale (EQ VAS). The EQ-5D-5L descriptive system comprises of 5 dimensions of health: mobility, self-care, usual activities, pain/discomfort and anxiety/depression. For each dimension, patients select one of 5 levels of severity: no problems, slight problems, moderate problems, severe problems, and extreme problems. The EQ VAS records the patient's self-rated health on a vertical visual analogue scale from 0 to 100 where the endpoints are labeled the worst and best imaginable health respectively.

In aspects, following treatment with a composition of the disclosure a subject reports an increased level of health as determined by EQ-5D-5L. In aspects, health is increased by at least about or at most about: 5, 10, 20, 30, 40, 50, 60, 70, 80, or 90 points as compared to a baseline level prior to treatment. In aspects, an EQ-5D-5L test is performed at any time before, during, or after treatment with a Tn3 scaffold of the disclosure.

Schirmer's Test

An assessment of the disclosure can be Schirmer's test. In aspects, a Schirmer's test without local anesthesia is performed. Schirmer's test measures lacrimal gland function. It utilizes calibrated strips of a non-toxic filter paper to measure the flow of tears. One end of the strip is placed within the lower eyelid. Both eyes need to be measured simultaneously. After the placement, participants are asked to keep their eyes gently closed for 5 minutes at which point the strips are removed from the eyelids and the extent of the wetting of each of the strips is recorded.

In aspects, following treatment with a composition of the disclosure increased wetting is detected as compared to a level of wetting prior to treatment or a baseline level. In aspects, wetting is increased by at least about or at most about 5%, 10%, 20%, 30%, 40%, or 50% as compared to baseline. In aspects, Schirmer′ test is performed at any time before, during, or after treatment with a Tn3 scaffold of the disclosure.

### Physician Global Impression of Severity (MDGIS)

In aspects, an assessment can comprise a MDGIS survey. The MDGIS represents an assessment of SS disease severity and is a 5-point categorical response scale (1-none, 2-mild, 3=moderate, 4=severe, or 5=very severe). In aspects, a MDGIS score can range from about 0 to about 5 or about 1 to about 5. In aspects, a MDGIS score can be from about 1, 2, 3, 4, or up to about 5. In aspects, a MDGIS score can be from about 0, 1, 2, 3, or up to about 4. In aspects, a MDGIS score may comprise assessing a subject in need thereof administered a Tn3 scaffold as compared to baseline. In aspects, a subject's MDGIS score is decreased as compared to an otherwise comparable method wherein the subject of the otherwise comparable method is not administered a Tn3 scaffold.

In aspects, treatment with a composition of the disclosure is effective in reducing a MDGIS score. In aspects, a MDGIS score is reduced by at least about: 1, 2, 3, 4, or 5 points. In aspects, a MDGIS score is reduced by up to about 1, 2, 3, 4, or 5 points. In aspects, a MDGIS score is reduced from about 1-5, 2-5, 1-4, or 3-5 points.

In aspects, a MDGIS survey can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a MDGIS survey can be made on about Day 1, Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, MDGIS is assessed at any time before, during, or after treatment with a Tn3 scaffold of the disclosure.

### Electrocardiogram (ECG)

In aspects, an assessment can comprise an ECG. A single 12-lead ECG with the subject in the supine position may be performed. Electrocardiogram measurement may be delayed by at least 10 minutes if performed after phlebotomy. Each ECG may include ventricular heart rate and intervals (PR, QRS, QT, RR).

In aspects, an ECG evaluation can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an ECG evaluation can be assessed on about Day 1, Day 169 (±7 days), or on about Day 337 (±7 days) post treatment initiation. In aspects, ECG is assessed at any time before, during, or after treatment with a Tn3 scaffold of the disclosure.

### Clinical Safety Laboratory Tests

In aspects, an assessment can comprise a clinical safety lab test. Blood and urine samples may be collected for laboratory safety tests using clinically acceptable methods and devices. Abnormal laboratory findings associated with the underlying disease may not be considered clinically significant.

All laboratory tests with values considered clinically significantly abnormal during participation in the study or within the protocol follow-up period after the last dose of a Tn3 scaffold may be repeated until the values return to normal or baseline or are no longer considered clinically significant. Additional tests may be performed at any time during the study.

In aspects, a clinical safety laboratory test can include hematology, clinical chemistry, urine testing (urinalysis), pregnancy testing, and other screening tests. Exemplary hematology parameters may include, but are not limited to, platelet count, red blood cell (RBC) count, RBC indices (mean corpuscular volume [MCV] and mean corpuscular hemoglobin [MCH]), white blood cell [WBC] count with differential (e.g., neutrophils, lymphocytes, monocytes, eosinophils, and/or basophils), hemoglobin, hematocrit, and immunoglobulins (IgG, IgM, and IgA). Exemplary clinical chemistry parameters may include, but are not limited to, blood urea nitrogen (BUN), potassium, creatinine, sodium, calcium, chloride, bicarbonate, phosphorous, glucose, aspartate aminotransferase (AST)/serum glutamic-oxaloacetic transaminase (SGOT), alanine aminotransferase (ALT)/serum glutamic-pyruvic transaminase (SPGT), alkaline phosphatase, bilirubin, gamma-glutamyltransferase, albumin, total protein, creatinine kinase, calculated estimated glomerular filtration rate (eGFR), uric acid, total cholesterol, low-density lipoprotein (LDL), high-density lipoprotein (HDL), and triglycerides. Exemplary urine testing parameters include, but are not limited to, specific gravity, pH, glucose, protein, blood, ketones, microscopic examination (e.g., crystals, cast, WBCs, RBCs), and protein: creatinine ratio. Exemplary pregnancy testing parameters may include, but are not limited to, serum beta human chorionic gonadotropin (β-hCG) pregnancy tests and urine pregnancy tests. Other screening test parameters may include, but are not limited to, follicle-stimulating hormone, serology (e.g., hepatitis B virus [HBV], HCV, human immunodeficiency virus-1 [HIV-1], HIV-2), severe acute respiratory syndrome coronavirus 2 (SARS-COV-2) rapid test, tuberculosis (TB) test (e.g., interferon gamma release assay [IGRA]), and a coagulation panel (e.g., prothrombin time, international normalized ratio [INR], partial thromboplastin time [PTT]). In aspects, a clinical safety lab test may comprise assessing a subject in need thereof administered a Tn3 scaffold as compared to baseline. In aspects, a subject's clinical safety lab test is decreased as compared to an otherwise comparable method wherein the subject of the otherwise comparable method is not administered a Tn3 scaffold. In aspects, a subject's clinical safety lab test is increased as compared to an otherwise comparable method wherein the subject of the otherwise comparable method is not administered a Tn3 scaffold.

In aspects, a clinical safety laboratory test can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a clinical safety laboratory test can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 15 (−3 days to +1 day), Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), Day 337 (±7 days), or on about Day 393 (±7 days) post treatment initiation. In aspects, clinical tests are conducted at any time before, during, or after treatment with a Tn3 scaffold of the disclosure.

### Immunogenicity

In aspects, an assessment can comprise determining a level of immunogenicity, if any, of an anti-Tn3 scaffold of the disclosure. Immunogenicity comprises determining the presence of an anti-drug antibody (ADA) to a Tn3 scaffold. The presence of ADA can be evaluated using a plasma sample from a subject administered a Tn3 scaffold. Plasma samples for ADA to a Tn3 scaffold of the disclosure may be taken prior to IP administration according to the visits and assessed using a validated immunoassay. In aspects, ADA are not detected post administration of a Tn3 scaffold. In aspects, ADA levels are reduced as compared to an otherwise comparable method wherein the subject of the otherwise comparable method is not administered a Tn3 scaffold, for example, the reduction may be about: 10%, 15%, 20%, 25%, 50%, 60%, 70%, 80%, 90%, 95%, 96%, 97%, 98%, 99%, or up to 100% as compared to ADA levels in an otherwise comparable method wherein the subject of the otherwise comparable method is not administered a Tn3 scaffold.

Antibodies to a Tn3 scaffold of the disclosure may be evaluated in plasma samples collected from all subjects. Additionally, plasma samples should also be collected at the final visit from subjects who discontinued administration of a Tn3 scaffold or were withdrawn from the study.

Plasma samples may be screened for antibodies binding to a Tn3 scaffold of the disclosure and the titer of confirmed positive samples may be reported. Other analyses may be performed to verify the stability of antibodies to a Tn3 scaffold of the disclosure and/or further characterize the immunogenicity of a Tn3 scaffold of the disclosure.

The detection and characterization of antibodies to a Tn3 scaffold of the disclosure may be performed using a validated assay method. Antibodies may be further characterized and/or evaluated for their ability to neutralize the activity of the study intervention(s). Samples may be stored for up to at least 15 years following the last subject's last visit for the study to enable further analysis of immune responses to a Tn3 scaffold of the disclosure. The number and percentage of subjects who develop ADA and ADA titer may be summarized by visit and by treatment group.

In aspects, an immunogenicity assessment can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an immunogenicity assessment can be made on about Day 1, Day 15 (−3 days to +1 day), Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), Day 337 (±7 days), or on about Day 393 (±7 days) post treatment initiation. In aspects, immunogenicity, or lack thereof, is assessed at any time before, during, or after treatment with a Tn3 scaffold of the disclosure.

### Ultrasound Imaging

In aspects, an assessment can comprise an ultrasound imaging. In aspects, an ultrasound imaging can be of one or more joints. In aspects, an ultrasound imaging can be of one or more salivary glands.

### Salivary Glands Ultrasound Imaging

Ultrasound measurement of the parotid and submandibular glands may be performed. A subject with significant glandular disease may be selected to participate. The glands above may be assessed in longitudinal and transverse planes with subjects in supine position. After image capture and assessment of quality, images will be scored and data summed. Echostructure of each gland on B-mode images may be scored on a 5-point scale (0 to 4) as previously described (Gazeau et al, 2018). Grading criteria will be as follows: Grade 0: normal homogenous gland; Grade 1: small hypoechoic areas with hyperechoic bands; Grade 2: multiple hypoechoic areas<2 mm; Grade 3: multiple hypoechoic areas 2 to 6 mm; Grade 4: multiple hypoechoic areas>6 mm.

At each time point assessed, 4 scores may be collected, one for each parotid and one for each submandibular gland. The Salivary Gland Ultrasound score for each assessment will be the sum of these grades.

### Joints Ultrasound Imaging

Ultrasound evaluation of small peripheral joints may be performed. Subjects with possible articular involvement may be selected. A modified German 7-joint US scoring system may be used (Backhaus et al, 2009). After image capture and assessment of quality, images will be transmitted to a scored and data summed.

Grayscale (GS) ultrasound may be performed on the following clinically dominant hand and foot joints: wrist (dorsal, palmar, and ulnar planes), second and third MCP (MCP2 and MCP3, palmar plane) and PIP (PIP2 and PIP3, palmar planes), and second and fifth metatarsophalangeal (MTP2 and MTP5, dorsal plane) joints. In GS, each joint and each plane may be semi-quantitatively scored on the following scale of 0 to 3 for synovitis: Grade 0=absence of synovitis; Grade 1=mild synovitis (small hypoechoic/anechoic line beneath the joint capsule); Grade 2=moderate synovitis (joint capsule is elevated parallel to joint), and; Grade 3=severe synovitis (maximal distention of joint capsule). The total GS for synovitis score will be the sum of each joint or plane, for a score of 0-27. In aspects, the total GS for synovitis score with be from about 0 to about 27, or from about 1 to about 27. In aspects, the total GS for synovitis score will be from about 0, 1, 2, 3, 4, 5, 6, 7, 8, 9, 10, 11, 12, 13, 14, 15, 16, 17, 18, 19, 20, 21, 22, 23, 24, 25, 26, or up to about 27.

Power doppler ultrasound will be performed on the following clinically dominant hand and foot joints: wrist (dorsal, palmar, and ulnar planes), second and third MCP (MCP2 and MCP3, palmar and dorsal planes) and PIP (PIP2 and PIP3, palmar and dorsal planes), and second and fifth metatarsophalangeal (MTP2 and MTP5, dorsal plane only) joints. Synovitis assessment using power doppler will be scored on a scale of 0 to 3 as follows: Grade 0=no IA color signal; Grade 1=mild synovitis (up to 3 color signals or 2 single and 1 confluent signal in IA space); Grade 2=moderate synovitis (greater than Grade 1 but color occupying less than 50% of IA space), and; Grade 3=severe synovitis (greater than 50% color in IA space). The total GS for synovitis score will be the sum of each joint or plane, for a score of 0-39. In aspects, a total GS score is from about 0 to about 39 or from about 1 to about 39. In aspects, a total GS score is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, up to about 39.

The total synovitis score will be the sum of GS and power doppler scores, for range of 0-66. In aspects, a total synovitis score is from about 0 to about 66 or from about 1 to about 66. In aspects, a total synovitis score is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, up to about 66.

In aspects, a synovitis score of the disclosure may comprise assessing a subject in need thereof administered a Tn3 scaffold as compared to baseline. In aspects, a synovitis score of the disclosure is decreased as compared to an otherwise comparable method wherein the subject of the otherwise comparable method is not administered a Tn3 scaffold.

In aspects, treatment with a composition of the disclosure is effective in reducing a synovitis score. In aspects, a synovitis score is reduced by at least about: 1, 2, 3, 4, 5, 6, 7, 8, 9, 10, 15, 20, 25, 30, 35, 40, 45, 50, 55, 60, 65, or 66 points. In aspects a synovitis score is reduced by up to about 1, 2, 3, 4, 5, 6, 7, 8, 9, 10, 15, 20, 25, 30, 35, 40, 45, 50, 55, 60, 65, or 66 points. In aspects, a synovitis score is reduced from about 1-5, 2-10, 5-10, 5-20, 10-20, 20-30, 30-40, 40-50, 50-60, or 55-66 points.

In aspects, an ultrasound imaging assessment can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an ultrasound imaging assessment can be made on about Day 1, Day 197 (±7 days), or on about Day 337 (±7 days) post treatment initiation. In aspects, imaging is completed at any time before, during, or after treatment with a Tn3 scaffold of the disclosure.

### Transcriptomics

In aspects, an assessment can comprise evaluation of transcriptomics. In aspects, RNA testing is performed. In aspects, RNA testing is performed to measure expression levels of genes associated with disease activity, specific cell types (e.g., plasma cell gene signature), T follicular helper gene signature, and signaling, e.g., the CD40L/CD40 pathway.

In aspects, blood RNA is used to measure the expression levels of genes associated with disease activity, specific cell types (e.g., plasma cell gene signature), T follicular helper gene signature, and signaling, including the CD40L/CD40 pathway. In aspects, a blood sample will be collected using the PAXgene Blood RNA System for the collection, transport, and storage of blood and stabilization of intracellular RNA in a closed tube and subsequent isolation and purification of intracellular RNA from whole blood for microarray analysis and quantitative polymerase chain reaction.

In aspects, expression levels of genes associated with disease activity by way of RNA analysis may be reduced by at least about 10%, 15%, 20%, 25%, 50%, 60%, 70%, 80%, 90%, 95%, 96%, 97%, 98%, 99%, or up to 100% as compared to an otherwise comparable method wherein the subject of the otherwise comparable method is not administered a Tn3 scaffold.

In aspects, a transcriptomics evaluation can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a transcriptomics evaluation can be assessed on about Day 1, Day 15 (−3 days to +1 day), Day 29 (±4 days), Day 85 (±7 days), Day 169 (±7 days), or on about Day 337 (±7 days) post treatment initiation. In aspects, transcriptomics are performed at any time before, during, or after treatment with a Tn3 scaffold of the disclosure.

### Genetic Analyses

In aspects, an assessment provided herein can comprise evaluation of genetics by way of DNA or RNA testing. In aspects, DNA testing is performed. In aspects, RNA testing is performed. In aspects, DNA testing can be performed to measure pharmacogenomic (single nucleotide polymorphism [SNP]) profiling of CD40 and other genes involved in the CD40/CD40L axis. In aspects, DNA is collected for epigenetics analysis, such as DNA methylation, of immune related genes. In aspects, an assessment comprises determining the sequence of genes associated with disease activity by way of whole blood DNA analysis.

In aspects, whole blood can be collected and may be used to evaluate gene sequences before, during, and after treatment with any of the compositions provided herein. Gene sequences found to be modulated by treatment may be analyzed in whole blood using quantitative methods. Samples may be used to examine gene sequences and their changes over time as assessed by NGS, Sanger Sequencing, or PCR.

In aspects, a genetic analysis evaluation can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a genetic evaluation is performed at any time before, during, or after treatment with a Tn3 scaffold of the disclosure.

### Pharmacokinetics

In aspects, a method provided herein can comprise determining a concentration of Tn3 scaffold in a subject in need thereof post administration. In aspects, a method comprises a pharmacokinetic assessment. In aspects, a sample is a blood sample or a plasma sample, or a combination of both. In aspects, a suitable assay to measure pharmacokinetics may comprise electrochemiluminescence (ECL) assay, a bead-based assay, a cell-based assay, and combinations thereof. In aspects, a sample may comprise plasma and the plasma is assessed for Tn3 scaffold concentration by measuring: maximum observed concentration (Cₘₐₓ), area under the concentration-time curve (AUC), clearance (CL), and terminal elimination half-life (t_{1/2}).

Samples will be used to evaluate the PK of a Tn3 scaffold of the disclosure. Samples collected for analyses of a Tn3 scaffold of the disclosure plasma concentration may also be used to evaluate safety or efficacy aspects during or after the study.

In aspects, a pharmacokinetic assessment can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an immunogenicity assessment can be made on about Day 1, Day 85 (±7 days), Day 169 (±7 days), Day 253 (±7 days), Day 337 (±7 days), or on about Day 393 (±7 days) post treatment initiation. In aspects, pharmacokinetics are assessed at any time before, during, or after treatment with a Tn3 scaffold of the disclosure.

### Pharmacodynamics

In aspects, an assessment can comprise evaluation of pharmacodynamics (PD). In aspects, an assessment can occur over a period of time. Whole blood, plasma, urine, saliva, and serum samples may be collected to evaluate PD of a Tn3 scaffold of the disclosure. In aspects, a sample may be assessed by a validated assay, including but not limited to, flow cytometry.

Samples may be collected to assess a level of a biomarker including but not limited to immunoglobulins (IgM, IgG, and IgA), sCD40L, CXCL13, β-2 microglobulin, high-sensitivity CRP, serum C3, C4, free light chains, peripheral blood mononuclear cells (PBMCs), anti-SSA, anti-SSB, cryoglobulins, and serum and urine immunofixation. In aspects, the immunoglobulins are plasma immunoglobulins. In aspects, a biomarker is a cellular biomarker such as one expressed by a subset of B-cells or T-cells. Exemplary B and T cell subsets include, but are not limited to, plasmablasts (e.g., CD27br/CD38br/IgD-subsets of CD19+ cells), precursor memory B cells (e.g., CD11cbr subsets of CD19+ cells), T follicular helper (Tfh) cells (e.g., CXCR5+/ICOS+ subsets of CD3+/CD4+ cells), proliferation of post-switch memory B cells (e.g., Ki67+ subsets of CD27br/IgD−/CD19+ cells), and/or proliferation of total T-cells (e.g., Ki67+ subsets of CD3+ cells). In aspects, an assessment comprises determining a level of one or more of: CD19, CD20, CD27, CD38, and CD138.

In aspects, a level of a biomarker is increased in a subject in need thereof post administration of a Tn3 scaffold of the disclosure. In aspects, a level of a biomarker is decreased in a subject in need thereof post administration of a Tn3 scaffold of the disclosure. In aspects, a reduction of biomarkers may be detected as compared to an otherwise comparable method wherein the subject of the otherwise comparable method is not administered a Tn3 scaffold. In aspects, elimination of a biomarker may be detected as compared to an otherwise comparable method wherein the subject of the otherwise comparable method is not administered a Tn3 scaffold as compared to an otherwise comparable method lacking the administering of a Tn3 scaffold. In aspects, reduction of a biomarker comprises at least about or at most about: 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 115-fold, 120-fold, 125-fold, 130-fold, 135-fold, 140-fold, 145-fold, 150-fold, 155-fold, 160-fold, 165-fold, 170-fold, 175-fold, 180-fold, 185-fold, 19-fold, 195-fold, 200-fold, 210-fol, 220-fold, 230-fold, 240-fold, 250-fold, 260-fold, 270-fold, 280-fold, 290-fold, or up to about 300-fold reduction as compared to an otherwise comparable method lacking the administering. In aspects, treatment efficacy of a Tn3 scaffold on a biomarker can be assessed over time using a suitable immunoassay. In aspects, effects of a Tn3 scaffold are assessed over time using a qualified immunoassay.

The disclosure provides for Tn3 scaffold-containing compositions that alter the CD40/CD40L pathway in a subject. The disclosure provides for Tn3 scaffold containing compositions that efficiently reduce or deplete soluble CD40L (sCD40L) in a subject. Because a Tn3 scaffold binds to and depletes a biomarker, the reduction or elimination of a biomarker can be used as a measure of treatment efficacy. In aspects, sCD40L is a measure of target engagement. In aspects, suitable assays to assess sCD40L levels may comprise flow cytometry, histology, immunohistochemistry, blood analysis, microscopy, PCR, ELISA, and combinations thereof.

The disclosure provides for Tn3 scaffold-containing compositions that efficiently reduce, eliminate, or inhibit major leukocyte populations (e.g., B lymphocytes), anti-SSA (Ro), anti-SSB (La), antinuclear antibodies, rheumatoid factor (RF), and combinations thereof. Suitable assays to assess leukocyte populations, anti-SSA (Ro), anti-SSB (La), antinuclear antibodies, and rheumatoid factor (RF) may comprise flow cytometry, histology, immunohistochemistry, blood analysis, microscopy, PCR, ELISA, and combinations thereof.

In aspects, Tn3 scaffolds of the disclosure may achieve at least about 10%, 15%, 20%, 25%, 50%, 60%, 70%, 80%, 90%, 95%, 96%, 97%, 98%, 99%, or up to about 100% reduction in CD40L as compared to an otherwise comparable method wherein the subject of the otherwise comparable method is not administered a Tn3 scaffold. Reduction of major leukocyte populations may persist for extended periods of time. In aspects, depletion of CD40L major leukocyte populations, anti-SSA (Ro), anti-SSB (La), antinuclear antibodies, and rheumatoid factor (RF), or a combination thereof, may persist for at least 1 day, at least 2 days, at least 3 days, at least 4 days, at least 5 days, at least 6 days, at least 7 days, at least 8 days, at least 9 days, at least 10 days, at least 15 days, at least 20 days, at least 25 days, or at least 30 days. In aspects, depletion of CD40L, major leukocyte populations, anti-SSA (Ro), anti-SSB (La), antinuclear antibodies, and rheumatoid factor (RF), or a combination thereof, may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In aspects, depletion of CD40L, major leukocyte populations, anti-SSA (Ro), anti-SSB (La), antinuclear antibodies, and rheumatoid factor (RF), or a combination thereof, may persist for at least 1 month, at least 2 months, at least 3 months, at least 4 months, at least 5 months, at least 6 months, at least 7 months, at least 8 months, at least 9 months, at least 10 months, at least 11 months, or at least 12 months.

In aspects, a pharmacodynamics assessment can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a pharmacodynamics assessment can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Day 15 (−3 days to +1 day), Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, pharmacodynamics are assessed at any time before, during, or after treatment with a Tn3 scaffold of the disclosure.

### Inflammatory Markers

In aspects, an assessment comprises determining a level of markers of inflammation. Exemplary markers of inflammation include but are not limited to: immunoglobulins (IgM, IgG, IgA), beta-2 microglobulin, C-reactive protein (CRP), CXCL13, serum C3, C4 and free light chains, cryoglobulins, and serum and urine immunofixation and combinations thereof. In aspects, whole blood, plasma, serum, and urine is collected to assess markers of inflammation. In aspects, a change as compared to a baseline is determined. In aspects, a change from baseline in levels of markers of inflammation (immunoglobulins, beta-2 microglobulin, CRP, CXCL13, serum C3, C4 and free light chains) is determined. In aspects, suitable assays to assess a level of inflammation comprise: ELISA, high sensitivity (hs)-CRP test, CRP test, Luminex, and combinations thereof. In aspects, administration of a Tn3 scaffold of the disclosure is effective in reducing a level of a biomarker of inflammation in a subject by at least about or at most about: 20%, 30%, 40%, 45%, 50%, 60%, 75%, 80%, 90%, 95%, 96%, 97%, 98%, 99%, or up to 100% as compared to of the levels of autoantibodies in an otherwise comparable method wherein the subject of the otherwise comparable method is not administered a Tn3 scaffold. In aspects, administration of a Tn3 scaffold of the disclosure is effective in reducing a level of a biomarker of inflammation in a subject by at least about or at most about: 3%-5%, 5%-10%, 10%-20%, or 5%-25% as compared to a baseline level prior to the administration.

In aspects, an assessment of inflammatory markers can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an assessment of inflammatory markers can be made on about Day 1, Day 15 (−3 days to +1 day), Day 29 (±4 days), Day 57 (±7 days), Day 85 (±7 days), Day 113 (±7 days), Day 141 (±7 days), Day 169 (±7 days), Day 197 (±7 days), Day 225 (±7 days), Day 253 (±7 days), Day 281 (±7 days), Day 309 (±7 days), or on about Day 337 (±7 days) post treatment initiation. In aspects, markers are assessed at any time before, during, or after treatment with a Tn3 scaffold of the disclosure.

### American College of Rheumatology/European Alliance of Associations for Rheumatology (ACR/EULAR)

In aspects, an assessment comprises an ACR/EULAR classification. In aspects, an assessment comprises a 2016 ACR/EULAR classification. In aspects, an assessment comprises a 2016 ACR/EULAR classification for primary Sjögren's Syndrome. In aspects, a classification comprises a score based on the following items: labial salivary gland with focal lymphocytic sialadenitis and focus score, Anti-SSA (Ro) positive, ocular staining score and/or van Bijsterveld score, Schirmer score, and a measure of unstimulated whole saliva flow rate. In aspects, a labial salivary gland with focal lymphocytic sialadenitis and focus score is >1. In aspects, an ocular staining score is ≥5 on at least one eye. In aspects, a van Bijsterveld score is ≥4. In aspects, a Schirmer score is ≤5 mm/5 min in at least one eye. In aspects, an unstimulated whole saliva flow rate is ≤0.1 mL/min. In aspects, one or more scores from an ACR/EULAR classification is weighted. In aspects, a labial salivary gland with focal lymphocytic sialadenitis and focus is weighted 3. In aspects, an ocular staining score is weighted 3. In aspects, a van Bijsterveld score is weighted 1. In aspects, a Schirmer score is weighted 1. In aspects, an unstimulated whole saliva flow rate is weighted 1. In aspects, a subject in need thereof has a combined weighed score of >4. In aspects, a subject in need thereof may be asked at least one of the following questions: 1) Have you had daily, persistent, troublesome dry eyes for more than 3 months?; 2) Do you have a recurrent sensation of sand or gravel in the eyes?; 3) Do you use tear substitutes more than 3 times a day?; 4) Have you had a daily feeling of dry mouth for more than 3 months?; 5) Do you frequently drink liquids to aid in swallowing dry food?

In aspects, an ACR/EULAR assessment can be made on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, an ACR/EULAR assessment can be made on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, or on about Day 0 post treatment initiation. In aspects, ACR/EULAR is assessed at any time before, during, or after treatment with a Tn3 scaffold of the disclosure.

### Ocular Surface Disease Index (OSDI)

In aspects, an assessment comprises an OSDI evaluation. In aspects, an OSDI evaluation comprises an OSDI questionnaire. The OSDI is effective in discriminating among normal, mild to moderate, and severe dry eye disease. In aspects, an OSDI evaluation evaluates three subsets: vision-related function, ocular symptoms, or environmental triggers, or combinations thereof.

In aspects, an OSDI evaluation produces a score. In aspects, an OSDI score ranges from about 0 to about 100:0 to 12 represents normal, 13-22 represents mild dry eye disease, 23-32 represents moderate dry eye disease, 33-100 represents severe dry eye disease. In aspects, an OSDI score ranges from about 0 to about 12, from about 13 to about 22, from about 23 to about 32, from about 33 to about 100, from about 20 to about 50, from about 60 to about 100, from about 40 to about 70, from about 10 to about 30, or from 5 to about 25.

In aspects, an OSDI score is decreased in a subject in need thereof post administration of a Tn3 scaffold of the disclosure. In aspects, an OSDI score may be decreased as compared to an otherwise comparable method wherein the subject of the otherwise comparable method is not administered a Tn3 scaffold as compared to an otherwise comparable method lacking the administering of a Tn3 scaffold. In aspects, an OSDI score is reduced by at least about or at most about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points as compared to an otherwise comparable method lacking the administering.

In aspects, OSDI evaluation can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, OSDI is assessed at any time before, during, or after treatment with a Tn3 scaffold of the disclosure.

### Sjögren's Tool for Assessing Response (STAR)

In aspects, an assessment can comprise the Sjögren's Tool for Assessing Response (STAR). STAR is a composite responder index that was developed by the NECESSITY consortium, supported by an international panel of pSS experts, scientists, methodologists, and patients to assess treatment efficacy based on improvement of disease activity (Seror et al, 2022). The STAR contains 5 domains: systemic activity, symptoms, lacrimal gland function, salivary gland function, and biomarkers of autoimmune activity. The domains are differently weighted. In aspects, a STAR domain comprises another assessment of the disclosure.

In aspects, a STAR survey is scored from about 1 to about 9. In aspects, a STAR survey is scored from about 1, 2, 3, 4, 5, 6, 7, 8, or about 9. In aspects, a STAR survey is scored at about 1 to about 3, at about 1 to about 5, at about 5 to about 9, at about 6 to about 9, at about 7 to about 9, or at about 4 to about 9. In aspects, a STAR survey is scored at about ≥5. In aspects, a STAR survey may comprise assessing a subject in need thereof administered a Tn3 scaffold of the disclosure as compared to baseline. In aspects, a subject's STAR score is increased as compared to an otherwise comparable method wherein the subject of the otherwise comparable method is not administered a Tn3 scaffold of the disclosure. In aspects, a subject in need thereof administered a Tn3 scaffold of the disclosure has an increased STAR score as compared to baseline. In aspects, a STAR score is increased by about 1, 2, 3, 4, 5, 6, 7, or 8 points.

In aspects, a STAR survey can be assessed on about −35 days, −34 days, −33 days, −32 days, −31 days, −30 days, −29 days, −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a STAR survey can be assessed on about 1 day, 15 days (+−3 to 1 days), 29 days±4 days, 57 days±7 days, 85 days±7 days, 113 days±7 days, 141±7 days, 169 days±7 days, 197 days±7 days, 225 days±7 days, 253 days±7 days, 281 days±7 days, 309 days±7 days, 337 days±7 days, or 421 days±7 days post treatment initiation. In aspects, a STAR survey is assessed at any time before, during, or after treatment with a Tn3 scaffold of the disclosure.

### Diary for Assessing Sjögren's Patient Reported Index (DASPRI)

In aspects, an assessment can comprise the Diary for Assessing Sjögren's Patient Reported Index (DASPRI). In aspects, DASPRI is also known as SS Symptom Diary (SSSD). DASPRI is a participant-completed questionnaire to measure the severity of core symptoms in patients with SS. It can have a recall period of about 24 hours. Subjects rate the severity of each symptom “at their worst” in domains comprising: dryness, fatigue (e.g., feeling of tiredness), and pain (joint or muscular pain in the arms and/or legs), using a numerical rating scale (ranging from 0 [no symptoms] to 10 [maximal imaginable severity]). The dryness domain assesses the severity of location-specific dryness (mouth, eyes, skin, and genital). In addition, subjects will be asked to rank their most bothersome dryness locations. In aspects, endpoints based on the DASPRI are defined as the average daily scores over a 7-day period.

In aspects, a DASPRI domain is scored from about 0 to about 10. In aspects, a DASPRI question is scored from about 0 to about 2, from about 1 to about 5, from about 2 to about 7, from about 0 to about 9, from about 1 to about 10, from about 8 to about 10, or from about 6 to about 10. In aspects, a DASPRI composite score is from about 0 to about 100. In aspects, a DASPRI composite score is about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100. In aspects, a DASPRI composite score is about 0 to about 10, about 5 to about 15, about 20 to about 20, about 15 to about 25, about 20 to about 30, about 20 to about 50, about 30 to about 60, about 40 to about 70, about 50 to about 80, about 60 to about 90, or about 70 to about 100.

In aspects, a DASPRI may comprise assessing a subject in need thereof administered a Tn3 scaffold of the disclosure as compared to baseline. In aspects, a subject's DASPRI score is reduced as compared to an otherwise comparable method wherein the subject of the otherwise comparable method is not administered a Tn3 scaffold of the disclosure. In aspects, a subject in need thereof administered a Tn3 scaffold of the disclosure has an reduced DASPRI as compared to baseline. In aspects, a DASPRI score is reduced by about 1, 2, 3, 4, 5, 10, 15, 20, 25, 30, 35, 40, 45, 50, 60, 70, 80, 90, 95, 96, 97, 98, 99, or 100.

In aspects, a DASPRI survey can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, or at least about 56 weeks post treatment initiation. In aspects, a DASPRI survey can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day and about once a week, twice a week, three times a week, four times a week, five times a week, six times a week, or seven times a week thereafter post-treatment initiation. In aspects, a DASPRI survey is assessed at any time before, during, or after treatment with a Tn3 scaffold of the disclosure.

### Pharmaceutical Compositions

In aspects, provided are pharmaceutical compositions. A pharmaceutical composition can comprise a Tn3 scaffold of the disclosure. In aspects, a pharmaceutical composition is part of a therapeutic regimen that comprises a Tn3 scaffold of the disclosure, and one or more additional therapeutics provided herein.

For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

In aspects, a pharmaceutical composition provided herein is administered via infusion. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours. In aspects, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. In aspects, infusion reactions can occur and include headache, nausea, somnolence, dyspnea, fever, myalgia, rash, or other symptoms. Potential risks associated with administration of a Tn3 scaffold are infection, redness, swelling, pain, and induration at the administration site. Prior to each IV infusion subjects may receive prophylaxis with IV methylprednisolone, oral diphenhydramine, and oral acetaminophen, or equivalent(s) to reduce the risk or severity of potential reactions.

In aspects, a treatment regime comprising a pharmaceutical composition may be dosed according to a body weight of a subject. In subjects who are determined obese (BMI>35) a practical weight may need to be utilized. In aspects, body surface area may be utilized to calculate a dosage.

In aspects, a pharmaceutical composition can be administered either alone or together with a pharmaceutically acceptable carrier or excipient, by any routes, and such administration can be carried out in both single and multiple dosages. More particularly, a pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, pharmaceutical formulations can be suitably sweetened and/or flavored by means of various agents of the type commonly employed for such purposes. Exemplary carriers and excipients can include dextrose, sodium phosphate monobasic, sodium phosphate dibasic, sodium phosphate monobasic/dibasic, sodium chloride (NaCl), sucrose, lactose, cellulose, xylitol, sorbitol, maltitol, gelatin, PEG, PVP, histidine/histidine hydrochloride, trehalose dihydrate, polysorbate 80, poloxamer 188 (pH 7.4) and any combination thereof. In aspects, a pharmaceutical composition used in the methods of the invention comprises: sodium phosphate monobasic/dibasic, sucrose, and poloxamer 188 (pH 7.4).

### NUMBERED EMBODIMENTS

### Embodiment Set 1

1. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has moderate-to-severe systemic disease activity as determined by the European Alliance of Associations for Rheumatology (EULAR) SS Disease Activity Index (ESSDAI).

2. The method of embodiment 1, wherein the subject has an ESSDAI score from 5-7, 5-10, 5-13, or 10-13 before the administration of the Tn3 scaffold.

3. The method of embodiment 2, wherein the subject has an ESSDAI score≥5.

4. The method of any one of embodiments 1-3, wherein the administration of the Tn3 scaffold is effective in reducing the EULAR Sjögren's Syndrome Patient Reported Index (ESSPRI) score following the administration.

5. The method of embodiment 4, wherein the ESSPRI score is reduced by at least 2, 3, 4, or 5 points.

6. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has a ESSPRI score≥5.

7. The method of embodiment 6, wherein the subject has an ESSDAI score of <5 before the administering of the Tn3 scaffold.

8. The method of any one of embodiments 6-7, wherein the subject has residual salivary gland function as defined by whole stimulated salivary flow greater than 0.1 mL/min.

9. The method of any one of embodiments 1-8, wherein a sample of the subject is positive for: anti-Ro autoantibodies, rheumatoid factor, or both anti-Ro autoantibodies and rheumatoid factor.

10. The method of any one of embodiments 1-9, wherein the dose is 1500 mg.

11. The method of any one of embodiments 1-9, wherein the dose is 3000 mg.

12. The method of any one of embodiments 2-11, wherein change from baseline in the ESSDAI is determined following the administration of the Tn3 scaffold.

13. The method of embodiment 12, wherein the change from baseline is determined every four weeks through week 48 following the administration of the Tn3 scaffold.

14. The method of any one of embodiments 2-13, wherein the administration of the Tn3 scaffold is effective in reducing the ESSDAI score of the subject following the administration.

15. The method of embodiment 14, wherein the ESSDAI score is reduced by at least 1, 2, 3, 4, or 5 points.

16. The method of embodiment 14, wherein the ESSDAI score is reduced by at least 5 points.

17. The method of any one of embodiments 1-16, wherein the administration of the Tn3 scaffold is effective in reducing a tender and swollen joint count baseline score of the subject following the administration.

18. The method of any one of embodiments 1-16, wherein the administration of the Tn3 scaffold is effective at reducing a Short Form 36 (SF-36) Health Survey baseline score of the subject following the administration.

19. The method of any one of embodiments 1-18, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

20. The method of any one of embodiments 1-19, wherein the administration of the Tn3 scaffold is effective at reducing a baseline level of a biomarker, and wherein the biomarker is selected from the group consisting of: plasma soluble CD40L, B-cells, serum CXCL13, rheumatoid factor autoantibodies, anti-Ro autoantibodies, and combinations thereof.

21. The method of any one of embodiments 1-20, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

22. The method of any one of embodiments 1-21, wherein expression levels of genes associated with SS are reduced in a blood sample of the subject by week 48 following the administration.

23. The method of any one of embodiments 1-22, wherein levels of B cells selected from the group consisting of: CD27+ memory B cells, marginal zone B cells, plasmablasts, plasma cells, and combinations thereof, are reduced in a blood sample of the subject by week 48 following the administration.

24. The method of any one of embodiments 1-23, wherein the Tn3 scaffold is administered as an loading dose and as a maintenance dose thereafter.

25. The method of embodiment 24, wherein the loading dose comprises administering the Tn3 scaffold once about every 2 weeks for at least 3 doses.

26. The method of embodiment 24, wherein the maintenance dose comprises administering the Tn3 scaffold once about every 4 weeks for at least 4 doses.

27. The method of embodiment 24, wherein the time between the last loading dose and the first maintenance dose is about 4 weeks.

28. The method of any one of embodiments 1-27, wherein the Tn3 scaffold is administered once about every 4 weeks, once about every 2 months, once about every 3 months, once about every 4 months, or once about every 6 months.

29. The method of any one of embodiments 1-28, wherein the Tn3 scaffold is administered for at least 4 doses.

30. The method of any one of embodiments 1-28, wherein the Tn3 scaffold is administered for at least 5 doses.

31. The method of any one of embodiments 1-30, wherein the Tn3 scaffold is administered intravenously, subcutaneously, orally, intramuscularly, intrathecally, sublingually, rectally, vaginally, cutaneously, systemically, topically, transdermally, or by way of inhalation.

32. The method of embodiment 31, wherein the Tn3 scaffold is administered intravenously.

33. The method of any one of embodiments 1-32, wherein the Tn3 scaffold comprises two CD40L-specific monomer subunits connected in tandem.

34. The method of embodiment 33, wherein the two CD40L-specific monomer subunits each comprise SEQ ID NO: 3.

35. The method of any of one of embodiments 33-34, wherein the CD40L-specific monomer subunits are connected by a linker.

36. The method of any one of embodiments 33-35, wherein at least one CD40L-specific monomer subunit is fused or conjugated to a polyethylene glycol (PEG) directly.

37. The method of any one of embodiments 33-35, wherein at least one CD40L-specific monomer subunit is fused or conjugated to a polyethylene glycol (PEG) via a linker.

38. The method of embodiment 37, wherein the linker comprises a peptide linker.

39. The method of embodiment 37, wherein the linker comprises SEQ ID NO: 7, SEQ ID NO: 8, SEQ ID NO: 9, or SEQ ID NO: 10.

40. The method of any one of embodiments 33-39, wherein at least one CD40L-specific monomer subunit is fused or conjugated to an albumin.

41. The method of embodiment 40, wherein the albumin is human serum albumin (HSA).

42. The method of embodiment 41, wherein the HSA is a variant HSA comprising SEQ ID NO: 4.

43. The method of any one of embodiments 24-42, wherein the loading and maintenance dose are the same.

44. The method of any one of embodiments 24-42, wherein the loading and maintenance dose are different.

45. The method of any one of embodiments 1-44, wherein the Tn3 scaffold comprises SEQ ID NO: 1.

46. The method of any one of embodiments 10-45, wherein the 1500 mg of the Tn3 scaffold is administered intravenously at weeks 0, 2, and 4, and then once every 4 weeks thereafter.

47. The method of any one of embodiments 11-45, wherein the 3000 mg of the Tn3 scaffold is administered intravenously at weeks 0, 4, and 12 and then once every 12 weeks thereafter.

48. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an ESSDAI score of ≥5 before the administration, and wherein the administration is effective at reducing the ESSDAI score as compared to an otherwise comparable subject dosed with placebo.

49. The method of embodiment 48, wherein the 1500 mg is administered intravenously at weeks 0, 2, and 4, and then once every 4 weeks thereafter.

50. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an ESSDAI score of ≥5 before the administration, and wherein the administration is effective at reducing the ESSDAI score as compared to an otherwise comparable subject dosed with placebo.

51. The method of embodiment 50, wherein the 3000 mg of the Tn3 scaffold is administered intravenously at weeks 0, 4, and 12 and then once every 12 weeks thereafter.

52. The method of any one of embodiments 48-51, wherein the ESSDAI score is reduced by 2 points, 3 points, 4 points, or 5 points.

53. The method of any one of embodiments 48-52, wherein the subject has a ESSDAI score from 5-7, 5-10, 5-13, or 10-13.

54. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

55. The method of embodiment 54, wherein the 1500 mg is administered intravenously at weeks 0, 2, and 4, and then once every 4 weeks thereafter.

56. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

57. The method of embodiment 56, wherein the 3000 mg of the Tn3 scaffold is administered intravenously at weeks 0, 4, and 12 and then once every 12 weeks thereafter.

58. The method of any one of embodiments 54-57, wherein the subject's ESSPRI score is from 5-6, 5-7, 6-8, 6-9, or 5-10 before the administration.

### Embodiment Set 2

1. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has moderate-to-severe systemic disease activity as determined by the European Alliance of Associations for Rheumatology (EULAR) SS Disease Activity Index (ESSDAI).

2. The method of embodiment 1, wherein the subject has an ESSDAI score from 5-7, 5-10, 5-13, or 10-13 before the administration of the Tn3 scaffold.

3. The method of embodiment 2, wherein the subject has an ESSDAI score≥5.

4. The method of any one of embodiments 1-4, wherein the administration of the Tn3 scaffold is effective in reducing the EULAR Sjögren's Syndrome Patient Reported Index (ESSPRI) score following the administration.

5. The method of embodiment 4, wherein the ESSPRI score is reduced by at least 2, 3, 4, or 5 points.

6. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has an ESSPRI score≥5.

7. The method of embodiment 6, wherein the subject has an ESSDAI score of <5 before the administering of the Tn3 scaffold.

8. The method of any one of embodiments 6-7, wherein the subject has residual salivary gland function as defined by whole stimulated salivary flow greater than 0.1 mL/min.

9. The method of any one of embodiments 1-8, wherein a sample of the subject is positive for: anti-Ro autoantibodies, rheumatoid factor, or both anti-Ro autoantibodies and rheumatoid factor. 10. The method of any one of embodiments 1-9, wherein the dose is 1500 mg.

11. The method of any one of embodiments 1-9, wherein the dose is 3000 mg.

12. The method of any one of embodiments 2-11, wherein change from baseline in the ESSDAI is determined following the administration of the Tn3 scaffold.

13. The method of embodiment 12, wherein the change from baseline is determined every four weeks through week 48 following the administration of the Tn3 scaffold.

14. The method of any one of embodiments 2-13, wherein the administration of the Tn3 scaffold is effective in reducing the ESSDAI score of the subject following the administration.

15. The method of embodiment 14, wherein the ESSDAI score is reduced by at least 1, 2, 3, 4, 5, 6, or 7 points.

16. The method of embodiment 14, wherein the ESSDAI score is reduced by at least 6 points.

17. The method of any one of embodiments 1-16, wherein the administration of the Tn3 scaffold is effective in reducing a tender and swollen joint count baseline score of the subject following the administration.

18. The method of any one of embodiments 1-16, wherein the administration of the Tn3 scaffold is effective at reducing a Short Form 36 (SF-36) Health Survey baseline score of the subject by week 48 following the administration.

19. The method of any one of embodiments 1-16, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

20. The method of any one of embodiments 1-18, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

21. The method of any one of embodiments 1-19, wherein the administration of the Tn3 scaffold is effective at reducing a baseline level of a biomarker, and wherein the biomarker is selected from the group consisting of: plasma soluble CD40L, B-cells, serum CXCL13, rheumatoid factor autoantibodies, anti-SSA autoantibodies (i.e., anti-Ro autoantibodies), anti-SSB autoantibodies (i.e. auto-La autoantibodies), and combinations thereof.

22. The method of any one of embodiments 1-20, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

23. The method of any one of embodiments 1-21, wherein expression levels of genes associated with SS are reduced in a blood sample of the subject by week 48 following the administration.

24. The method of any one of embodiments 1-22, wherein levels of B cells selected from the group consisting of: CD27+ memory B cells, marginal zone B cells, plasmablasts, plasma cells, and combinations thereof, are reduced in a blood sample of the subject by week 48 following the administration.

25. The method of any one of embodiments 1-23, wherein the Tn3 scaffold is administered as an loading dose and as a maintenance dose thereafter.

26. The method of embodiment 24, wherein the loading dose comprises administering the Tn3 scaffold once about every 2 weeks for at least 3 doses.

27. The method of embodiment 24, wherein the maintenance dose comprises administering the Tn3 scaffold once about every 4 weeks for at least 4 doses.

28. The method of embodiment 24, wherein the time between the last loading dose and the first maintenance dose is about 4 weeks.

29. The method of any one of embodiments 1-27, wherein the Tn3 scaffold is administered once about every 4 weeks, once about every 2 months, once about every 3 months, once about every 4 months, or once about every 6 months.

30. The method of any one of embodiments 1-28, wherein the Tn3 scaffold is administered for at least 4 doses.

31. The method of any one of embodiments 1-28, wherein the Tn3 scaffold is administered for at least 5 doses.

32. The method of any one of embodiments 1-30, wherein the Tn3 scaffold is administered intravenously, subcutaneously, orally, intramuscularly, intrathecally, sublingually, rectally, vaginally, cutaneously, systemically, topically, transdermally, or by way of inhalation.

33. The method of embodiment 31, wherein the Tn3 scaffold is administered intravenously.

34. The method of any one of embodiments 1-32, wherein the Tn3 scaffold comprises two CD40L-specific monomer subunits connected in tandem.

35. The method of embodiment 33, wherein the two CD40L-specific monomer subunits each comprise SEQ ID NO: 3.

36. The method of any of one of embodiments 33-34, wherein the CD40L-specific monomer subunits are connected by a linker.

37. The method of any one of embodiments 33-35, wherein at least one CD40L-specific monomer subunit is fused or conjugated to a polyethylene glycol (PEG) directly.

38. The method of any one of embodiments 33-35, wherein at least one CD40L-specific monomer subunit is fused or conjugated to a polyethylene glycol (PEG) via a linker.

39. The method of embodiment 37, wherein the linker comprises a peptide linker.

40. The method of embodiment 37, wherein the linker comprises SEQ ID NO: 7, SEQ ID NO: 8, SEQ ID NO: 9, or SEQ ID NO: 10.

41. The method of any one of embodiments 33-39, wherein at least one CD40L-specific monomer subunit is fused or conjugated to an albumin.

42. The method of embodiment 40, wherein the albumin is human serum albumin (HSA).

43. The method of embodiment 41, wherein the HSA is a variant HSA comprising SEQ ID NO: 4.

44. The method of any one of embodiments 24-42, wherein the loading and maintenance dose are the same.

45. The method of any one of embodiments 24-42, wherein the loading and maintenance dose are different.

46. The method of any one of embodiments 1-44, wherein the Tn3 scaffold comprises SEQ ID NO: 1.

47. The method of any one of embodiments 10-45, wherein the 1500 mg of the Tn3 scaffold is administered intravenously at weeks 0, 2, and 4, and then every 4 weeks thereafter.

48. The method of any one of embodiments 11-45, wherein the 3000 mg of the Tn3 scaffold is administered intravenously at weeks 0, 2, and 4, and then every 4 weeks thereafter.

49. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an ESSDAI score of ≥5 before the administration, and wherein the administration is effective at reducing the ESSDAI score as compared to an otherwise comparable subject dosed with placebo.

50. The method of embodiment 48, wherein the 1500 mg is administered intravenously at weeks 0, 2, and 4, and then once every 4 weeks thereafter.

51. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an ESSDAI score of ≥5 before the administration, and wherein the administration is effective at reducing the ESSDAI score as compared to an otherwise comparable subject dosed with placebo.

52. The method of embodiment 50, wherein the 3000 mg of the Tn3 scaffold is administered intravenously at weeks 0, 4, and 12 and then once every 12 weeks thereafter.

53. The method of any one of embodiments 48-51, wherein the ESSDAI score is reduced by 2 points, 3 points, 4 points, 5 points, 6 points or 7 points following the administration.

54. The method of any one of embodiments 48-51, wherein the subject has a ESSDAI score from 5-7, 5-10, 5-13, or 10-13 before the administration.

55. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

56. The method of embodiment 54, wherein the 1500 mg is administered intravenously at weeks 0, 2, and 4, and then once every 4 weeks thereafter.

57. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

58. The method of embodiment 56, wherein the 3000 mg of the Tn3 scaffold is administered intravenously at weeks 0, 4, and 12 and then once every 12 weeks thereafter.

59. The method of any one of embodiments 54-57, wherein the subject's ESSPRI score is from 5-6, 5-7, 6-8, 6-9, or 5-10 before the administration.

60. The method of any one of embodiments 5-58, wherein the administration is effective in achieving at least a 0.6 point, 0.8 point, 1 point, 1.8 point, or 2 point reduction in ESSPRI score as compared to baseline.

61. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject is positive for anti-SSA/Ro antibodies.

62. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject is positive for RF antibodies.

63. The method of any one of embodiments 1-60, wherein the administration is effective in achieving a ≥5%, 10%, 15%, 20%, 40%, or 60% reduction in ESSPRI score as compared to an otherwise comparable subject administered placebo or as compared to a baseline level of the subject.

### Embodiment Set 3

1. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has moderate-to-severe systemic disease activity as determined by the European Alliance of Associations for Rheumatology (EULAR) SS Disease Activity Index (ESSDAI).

2. The method of embodiment 1, wherein the subject has an ESSDAI score from 5-7, 5-10, 5-13, or 10-13 before the administration of the Tn3 scaffold.

3. The method of embodiment 2, wherein the subject has an ESSDAI score≥5.

4. The method of any one of embodiments 1-3, wherein the administration of the Tn3 scaffold is effective at reducing a EULAR Sjögren's Syndrome Patient Reported Index (ESSPRI) score following the administration.

5. The method of embodiment 4, wherein the ESSPRI score is reduced by at least 2, 3, 4, or 5 points.

6. The method of any one of embodiments 1-3, wherein the administration of the Tn3 scaffold is effective at reducing a DASPRI score.

7. The method of embodiment 6, wherein the DASPRI score is reduced by at least about 5, 10, 15, or 20 points.

8. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg to about 3000 mg, and wherein the subject has an ESSPRI score≥5.

9. The method of embodiment 8, wherein the subject has an ESSDAI score of <5 before the administering of the Tn3 scaffold.

10. The method of any one of embodiments 8-9, wherein the subject has residual salivary gland function as defined by whole stimulated salivary flow greater than 0.1 mL/min.

11. The method of any one of embodiments 1-10, wherein a sample of the subject is positive for: anti-Ro autoantibodies, rheumatoid factor autoantibodies, or both anti-Ro autoantibodies and rheumatoid factor autoantibodies.

12. The method of any one of embodiments 1-11, wherein the dose is 1500 mg. 13. The method of any one of embodiments 1-11, wherein the dose is 3000 mg.

14. The method of any one of embodiments 2-13, wherein change from baseline in the ESSDAI is determined following the administration of the Tn3 scaffold.

15. The method of embodiment 14, wherein the change from baseline is determined every four weeks through week 48 following the administration of the Tn3 scaffold.

16. The method of any one of embodiments 2-15, wherein the administration of the Tn3 scaffold is effective in reducing the ESSDAI score of the subject following the administration.

17. The method of embodiment 16, wherein the ESSDAI score is reduced by at least 1, 2, 3, 4, 5, 6, or 7 points.

18. The method of embodiment 16, wherein the ESSDAI score is reduced by at least 6 points.

19. The method of any one of embodiments 8-18, wherein the administration of the Tn3 scaffold is effective at reducing a DASPRI score.

20. The method of embodiment 19, wherein the DASPRI score is reduced by at least about 5, 10, 15, or 20 points.

21. The method of any one of embodiments 1-20, wherein the administration of the Tn3 scaffold is effective in reducing a tender and swollen joint count baseline score of the subject following the administration.

22. The method of any one of embodiments 1-21, wherein the administration of the Tn3 scaffold is effective at reducing a Short Form 36 (SF-36) Health Survey baseline score of the subject by week 48 following the administration.

23. The method of any one of embodiments 1-22, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

24. The method of any one of embodiments 1-23, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

25. The method of any one of embodiments 1-24, wherein the administration of the Tn3 scaffold is effective at reducing a baseline level of a biomarker, and wherein the biomarker is selected from the group consisting of: plasma soluble CD40L, B-cells, serum CXCL13, rheumatoid factor autoantibodies, anti-SSA autoantibodies (i.e., anti-Ro autoantibodies), anti-SSB autoantibodies (i.e. auto-La autoantibodies), and combinations thereof.

26. The method of any one of embodiments 1-25, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

27. The method of any one of embodiments 1-26, wherein expression levels of genes associated with SS are reduced in a blood sample of the subject by week 48 following the administration.

28. The method of any one of embodiments 1-27, wherein levels of B cells selected from the group consisting of: CD27+ memory B cells, marginal zone B cells, plasmablasts, plasma cells, and combinations thereof, are reduced in a blood sample of the subject by week 48 following the administration.

29. The method of any one of embodiments 1-28, wherein the Tn3 scaffold is administered as an loading dose and as a maintenance dose thereafter.

30. The method of embodiment 29, wherein the loading dose comprises administering the Tn3 scaffold once about every 2 weeks for at least 3 doses.

31. The method of embodiment 30, wherein the maintenance dose comprises administering the Tn3 scaffold once about every 4 weeks for at least 4 doses.

32. The method of embodiment 31, wherein the time between the last loading dose and the first maintenance dose is about 4 weeks.

33. The method of any one of embodiments 1-31, wherein the Tn3 scaffold is administered once about every 4 weeks, once about every 2 months, once about every 3 months, once about every 4 months, or once about every 6 months.

34. The method of any one of embodiments 1-33, wherein the Tn3 scaffold is administered for at least 4 doses.

35. The method of any one of embodiments 1-33, wherein the Tn3 scaffold is administered for at least 5 doses.

36. The method of any one of embodiments 1-35, wherein the Tn3 scaffold is administered intravenously, subcutaneously, orally, intramuscularly, intrathecally, sublingually, rectally, vaginally, cutaneously, systemically, topically, transdermally, or by way of inhalation.

37. The method of embodiment 36, wherein the Tn3 scaffold is administered intravenously.

38. The method of any one of embodiments 1-37, wherein the Tn3 scaffold comprises two CD40L-specific monomer subunits connected in tandem.

39. The method of embodiment 38, wherein the two CD40L-specific monomer subunits each comprise SEQ ID NO: 3.

40. The method of any of one of embodiments 38-39, wherein the CD40L-specific monomer subunits are connected by a linker.

41. The method of any one of embodiments 38-40, wherein at least one CD40L-specific monomer subunit is fused or conjugated to a polyethylene glycol (PEG) directly.

42. The method of any one of embodiments 38-40, wherein at least one CD40L-specific monomer subunit is fused or conjugated to a polyethylene glycol (PEG) via a linker.

43. The method of embodiment 42, wherein the linker comprises a peptide linker.

44. The method of embodiment 43, wherein the linker comprises SEQ ID NO: 7, SEQ ID NO: 8, SEQ ID NO: 9, or SEQ ID NO: 10.

45. The method of any one of embodiments 38-40, wherein at least one CD40L-specific monomer subunit is fused or conjugated to an albumin.

46. The method of embodiment 45, wherein the albumin is human serum albumin (HSA).

47. The method of embodiment 46, wherein the HSA is a variant HSA comprising SEQ ID NO: 4.

48. The method of any one of embodiments 29-47, wherein the loading and maintenance dose are the same.

49. The method of any one of embodiments 29-47, wherein the loading and maintenance dose are different.

50. The method of any one of embodiments 1-49, wherein the Tn3 scaffold comprises SEQ ID NO: 1.

51. The method of any one of embodiments 12-50, wherein the 1500 mg of the Tn3 scaffold is administered intravenously at weeks 0, 2, and 4, and then every 4 weeks thereafter.

52. The method of any one of embodiments 13-50, wherein the 3000 mg of the Tn3 scaffold is administered intravenously at weeks 0, 2, and 4, and then every 4 weeks thereafter.

53. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an ESSDAI score of ≥5 before the administration, and wherein the administration is effective at reducing the ESSDAI score as compared to an otherwise comparable subject dosed with placebo.

54. The method of embodiment 49, wherein the 1500 mg is administered intravenously at weeks 0, 2, and 4, and then once every 4 weeks thereafter.

55. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an ESSDAI score of ≥5 before the administration, and wherein the administration is effective at reducing the ESSDAI score as compared to an otherwise comparable subject dosed with placebo.

56. The method of embodiment 55, wherein the 3000 mg of the Tn3 scaffold is administered intravenously at weeks 0, 4, and 12 and then once every 12 weeks thereafter.

57. The method of any one of embodiments 49-56, wherein the ESSDAI score is reduced by 2 points, 3 points, 4 points, 5 points, 6 points or 7 points following the administration.

58. The method of any one of embodiments 49-57, wherein the subject has a ESSDAI score from 5-7, 5-10, 5-13, or 10-13 before the administration.

59. A method of treating Sjögren's syndrome (SS) in a subject in need thereof comprising: administering a Tn3 scaffold that comprises SEQ ID NO: 1 to the subject, wherein the Tn3 scaffold is administered at a dose of about 1500 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

60. The method of embodiment 59, wherein the 1500 mg is administered intravenously at weeks 0, 2, and 4, and then once every 4 weeks thereafter.

61. A method of treating Sjögren's syndrome (SS) in a subject in need thereof comprising: administering a Tn3 scaffold that comprises SEQ ID NO: 1 to the subject, wherein the Tn3 scaffold is administered at a dose of about 3000 mg, wherein the subject has an European Alliance of Associations for Rheumatology (EULAR) SS Disease Activity Index (ESSDAI) score<5, and wherein the subject has a EULAR Sjögren's Syndrome Patient Reported Index (ESSPRI) score≥5 before the administration.

62. The method of embodiment 61, wherein the 3000 mg of the Tn3 scaffold is administered intravenously at weeks 0, 4, and 12 and then once every 12 weeks thereafter.

63. The method of any one of embodiments 59-62, wherein the subject's ESSPRI score is from 5-6, 5-7, 6-8, 6-9, or 5-10 before the administration.

64. The method of any one of embodiments 4-63, wherein the administration is effective in achieving at least a 0.6 point, 0.8 point, 1 point, 1.8 point, or 2 point reduction in ESSPRI score as compared to baseline.

65. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject is positive for anti-SSA/Ro antibodies.

66. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject is positive for RF antibodies.

67. The method of any one of embodiments 1-66, wherein the administration is effective in achieving a ≥5%, 10%, 15%, 20%, 40%, or 60% reduction in ESSPRI score as compared to an otherwise comparable subject administered placebo or as compared to a baseline level of the subject.

68. The method of any one of embodiments 1-67, wherein the administration is effective in achieving a ≥5%, 10%, 15%, 20%, 40%, or 60% reduction in DASPRI score as compared to an otherwise comparable subject administered placebo or as compared to a baseline level of the subject.

### Embodiment Set 4

1. A Tn3 scaffold for use in the treatment of Sjögren's syndrome (SS), the Tn3 scaffold comprising a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered to a subject at a dose of about 1500 mg once every 4 weeks, wherein the subject has moderate-to-severe systemic disease activity as determined by the European Alliance of Associations for Rheumatology (EULAR) SS Disease Activity Index (ESSDAI) of ≥5.

2. The Tn3 scaffold for use according to embodiment 1, wherein prior to the once every 4 week dosing, the subject was administered at least 3 loading doses.

3. The Tn3 scaffold for use according to embodiment 2, wherein the at least 3 loading doses occur at weeks 0, 2, and 4.

4. A Tn3 scaffold for use in the treatment of Sjögren's syndrome (SS), the Tn3 scaffold comprising a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered to a subject at a dose of about 3000 mg once every 12 weeks, wherein the subject has moderate-to-severe systemic disease activity as determined by the European Alliance of Associations for Rheumatology (EULAR) SS Disease Activity Index (ESSDAI) of ≥5.

5. The Tn3 scaffold for use according to embodiment 4, wherein prior to the once every 12 week dosing, the subject was administered at least 3 loading doses.

6. The Tn3 scaffold for use according to embodiment 5, wherein the at least 3 loading doses occur at week 0, 4, and 12.

7. A Tn3 scaffold for use in the treatment of Sjögren's syndrome (SS), the Tn3 scaffold comprising a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered to a subject at a dose of about 1500 mg once every 4 weeks, wherein the subject has moderate-to-severe symptom state defined by ESSPRI score≥5, with a low systemic disease activity defined by ESSDAI score<5.

8. The Tn3 scaffold for use according to embodiment 7, wherein prior to the once every 4 week dosing, the subject was administered at least 3 loading doses.

9. The Tn3 scaffold for use according to embodiment 8, wherein the at least 3 loading doses occur at week 0, 2, and 4.

10. A Tn3 scaffold for use in the treatment of Sjögren's syndrome (SS), the Tn3 scaffold comprising a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered to a subject at a dose of about 3000 mg once every 12 weeks, wherein the subject has moderate-to-severe symptom state defined by ESSPRI score≥5, with a low systemic disease activity defined by ESSDAI score<5.

11. The Tn3 scaffold for use according to embodiment 10, wherein prior to the once every 12 week dosing, the subject was administered at least 3 loading doses.

12. The Tn3 scaffold for use according to embodiment 11, wherein the at least 3 loading doses occur at week 0, 4, and 12.

13. The Tn3 scaffold for use according to any one of embodiments 1-5, wherein the subject has an ESSDAI score from 5-7, 5-10, 5-13, or 10-13 before the administration of the Tn3 scaffold.

14. The Tn3 scaffold for use according to any one of embodiments 1-5, wherein the administration is effective at reducing the ESSDAI.

15. The Tn3 scaffold for use according to embodiment 14, wherein the ESSDAI score is reduced by at least 1, 2, 3, 4, 5, 6, or 7 points.

16. The Tn3 scaffold for use according to embodiment 15, wherein the ESSDAI score is reduced by at least 6 points.

17. The Tn3 scaffold for use according to any one of embodiments 13-16, wherein the ESSDAI score is reduced by at least 1 month, 2 months, 3 months, 4 months, 5 months, 6 months, 7 months, 8 months, 9 months, 10 months, 11 months, or 1 year following administration of the first loading dose as compared to a baseline ESSDAI score.

18. The Tn3 scaffold for use according to embodiment 17, wherein the ESSDAI score is reduced by 11 months following the administration of the first loading dose.

19. The Tn3 scaffold for use according to any one of embodiments 7-12, wherein subject comprises whole stimulated salivary flow>0.1 mL/min at baseline.

20. The Tn3 scaffold for use according to embodiment 19, wherein the ESSPRI score is reduced by at least about 1.5, 2, 3, 4, or 5 points following the week 0 loading dose administration.

21. The Tn3 scaffold for use according to any one of embodiments 7-12, wherein a DASPRI score is reduced following the administration.

22. The Tn3 scaffold for use according to embodiment 21, wherein the DASPRI score is reduced by at least about 5, 10, 15, or 20 points following the week 0 loading dose administration.

23. The Tn3 scaffold for use according to any one of embodiments 1-22, wherein the administration of the Tn3 scaffold is effective in reducing a tender and swollen joint count baseline score of the subject following the administration.

24. The Tn3 scaffold for use according to any one of embodiments 1-23, wherein the administration of the Tn3 scaffold is effective at reducing a Short Form 36 (SF-36) Health Survey baseline score of the subject following the administration.

25. The Tn3 scaffold for use according to any one of embodiments 1-22, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

24. The Tn3 scaffold for use according to any one of embodiments 1-23, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

25. The Tn3 scaffold for use according to any one of embodiments 1-24, wherein the administration of the Tn3 scaffold is effective at reducing a baseline level of a biomarker, and wherein the biomarker is selected from the group consisting of: plasma soluble CD40L, Ki67+CD27+ memory B cell, plasmablast, CD11c bright/high B cell, CD3, CD4, CD8, Tfh cell, serum CXCL13, rheumatoid factor, anti-SSA autoantibodies (i.e., anti-Ro autoantibodies), anti-SSB autoantibodies (i.e. anti-La autoantibodies), and combinations thereof.

26. The Tn3 scaffold for use according to any one of embodiments 1-25, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

27. The Tn3 scaffold for use according to any one of embodiments 1-26, wherein expression levels of genes associated with SS are reduced in a blood sample of the subject by week 48 following the administration.

28. The Tn3 scaffold for use according to any one of embodiments 1-27, wherein levels of B cells selected from the group consisting of: CD27+ memory B cells, marginal zone B cells, plasmablasts, plasma cells, and Combinations thereof, are reduced in a blood sample of the subject by week 48 following the administration.

29. The Tn3 scaffold for use according to any one of embodiments 1-28, wherein the Tn3 scaffold is administered intravenously.

30. The Tn3 scaffold for use according to any one of embodiments 1-29, wherein the Tn3 scaffold comprises two CD40L-specific monomer subunits connected in tandem.

31. The Tn3 scaffold of embodiment 30, wherein the two CD40L-specific monomer subunits each comprise SEQ ID NO: 3.

32. The Tn3 scaffold for use according to any of one of embodiments 30-31, wherein the CD40L-specific monomer subunits are connected by a linker.

33. The Tn3 scaffold for use according to any one of embodiments 30-32, wherein at least one CD40L-specific monomer subunit is fused or conjugated to a polyethylene glycol (PEG) directly.

34. The Tn3 scaffold for use according to any one of embodiments 30-33, wherein at least one CD40L-specific monomer subunit is fused or conjugated to a polyethylene glycol (PEG) via a linker.

35. The Tn3 scaffold for use according to embodiment 34, wherein the linker comprises a peptide linker.

36. The Tn3 scaffold for use according to embodiment 35, wherein the linker comprises SEQ ID NO: 7, SEQ ID NO: 8, SEQ ID NO: 9, or SEQ ID NO: 10.

37. The Tn3 scaffold for use according to any one of embodiments 30-36, wherein at least one CD40L-specific monomer subunit is fused or conjugated to an albumin.

38. The Tn3 scaffold for use according to embodiment 37, wherein the albumin is human serum albumin (HSA).

39. The Tn3 scaffold for use according to embodiment 38, wherein the HSA is a variant HSA comprising SEQ ID NO: 4.

40. The Tn3 scaffold for use according to any one of embodiments 1-39, wherein the Tn3 scaffold comprises SEQ ID NO: 1.

### Embodiment Set 5

1. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg once every 4 weeks, wherein the subject has moderate-to-severe systemic disease activity of 25 as determined by European Alliance of Associations for Rheumatology (EULAR) SS Disease Activity Index (ESSDAI).

2. The method of embodiment 1, wherein prior to the once every 4 week dosing, the subject was administered at least 3 loading doses of 1500 mg each.

3. The method of embodiment 2, wherein the at least 3 loading doses are administered at week 0, 2, and 4.

4. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 3000 mg once every 12 weeks, wherein the subject has moderate-to-severe systemic disease activity of ≥5 as determined by European Alliance of Associations for Rheumatology (EULAR) SS Disease Activity Index (ESSDAI).

5. The method of embodiment 4, wherein prior to the once every 12 week dosing, the subject was administered at least 3 loading doses of 3000 mg each.

6. The method of embodiment 5, wherein the at least 3 loading doses are administered at week 0, 4, and 12.

7. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg once every 4 weeks, wherein the subject has moderate-to-severe symptom state defined by ESSPRI score of ≥5, with a low systemic disease activity defined by ESSDAI score of <5, and wherein a Diary for Assessing Sjögren's Patient Reported Index (DASPRI) score is reduced in the subject following the administration.

8. The method of embodiment 7, wherein prior to the once every 4 week dosing, the subject was administered at least 3 loading doses of 1500 mg each.

9. The method of embodiment 8, wherein the at least 3 loading doses are administered at weeks 0, 2, and 4.

10. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 3000 mg once every 12 weeks, wherein the subject has moderate-to-severe symptom state defined by ESSPRI score≥5, with a low systemic disease activity defined by ESSDAI score<5, and wherein a Diary for Assessing Sjögren's Patient Reported Index (DASPRI) score is reduced in the subject following the administration.

11. The method of embodiment 10, wherein prior to the once every 12 week dosing, the subject was administered at least 3 loading doses of 3000 mg each.

12. The method of embodiment 11, wherein the at least 3 loading doses are administered at week 0, 4, and 12.

13. The method of any one of embodiments 1-5, wherein the subject has an ESSDAI score from 5-7, 5-10, 5-13, or 10-13 before the administration of the Tn3 scaffold.

14. The method of any one of embodiments 1-6, wherein the administration is effective at reducing the ESSDAI score.

15. The method of embodiment 14, wherein the ESSDAI score is reduced by at least 1, 2, 3, 4, 5, 6, or 7 points.

16. The method of embodiment 15, wherein the ESSDAI score is reduced by at least 6 points.

17. The method of any one of embodiments 15-16, wherein the ESSDAI score is reduced by at least 1 month, 2 months, 3 months, 4 months, 5 months, 6 months, 7 months, 8 months, 9 months, 10 months, 11 months, or 1 year following administration of the first loading dose as compared to a baseline ESSDAI score.

18. The method of embodiment 17, wherein the ESSDAI score is reduced by 11 months following the administration of the first loading dose.

19. The method of any one of embodiments 7-18, wherein salivary gland function in the subject is improved following administration of the Tn3 scaffold by about 3 weeks, 1 month, 2 months, or 4 months following the administration.

20. The method of embodiment 19, wherein salivary gland function is determined by whole stimulated salivary flow.

21. The method of any one of embodiments 9-20, wherein the ESSPRI score is reduced by at least about 1.5, 2, 3, 4, or 5 points following the loading dose administrations.

22. The method of any one of embodiments 7-21, wherein the DASPRI score is reduced by 2 weeks, 4 weeks, 1 month, 2 months, 3 months, 4 months, 5 months, or 6 months following the administration.

23. The method of embodiment 22, wherein the DASPRI score is reduced by at least about 5, 10, 15, or 20 points following the loading dose administrations.

24. The method of any one of embodiments 1-23, wherein the administration of the Tn3 scaffold is effective at reducing a tender and swollen joint count baseline score of the subject following the administration.

25. The method of any one of embodiments 1-24, wherein the administration of the Tn3 scaffold is effective at reducing a Short Form 36 (SF-36) Health Survey baseline score of the subject following the administration.

26. The method of any one of embodiments 1-25, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

27. The method of any one of embodiments 1-26, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

28. The method of any one of embodiments 1-27, wherein the administration of the Tn3 scaffold is effective at reducing a baseline level of a biomarker, and wherein the biomarker is selected from the group consisting of: plasma soluble CD40L, Ki67+CD27+ memory B cell, CD19, CD20, CD27, CD38, CD138, CD11c bright/high B cell, CD3, CD4, CD8, Tfh cell, serum CXCL13, rheumatoid factor, anti-SSA autoantibodies (i.e., anti-Ro autoantibodies), anti-SSB autoantibodies (i.e. anti-La autoantibodies), and combinations thereof.

29. The method of any one of embodiments 1-28, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

30. The method of any one of embodiments 1-29, wherein expression levels of genes, or levels of proteins encoded by the genes, associated with SS are reduced in a blood sample of the subject by week 48 following the administration.

31. The method of any one of embodiments 1-30, wherein levels of B cells selected from the group consisting of: CD27+ memory B cells, marginal zone B cells, plasmablasts, plasma cells, and combinations thereof, are reduced in a blood sample of the subject by week 48 following the administration.

32. The method of any one of embodiments 1-31, wherein the subject is positive for anti-Ro autoantibodies, rheumatoid factor (RF), or both anti-Ro autoantibodies and RF.

33. The method of any one of embodiments 1-32, wherein the Tn3 scaffold is administered intravenously.

34. The method of any one of embodiments 1-33, wherein the Tn3 scaffold comprises two CD40L-specific monomer subunits connected in tandem.

35. The method of embodiment 34, wherein the two CD40L-specific monomer subunits each comprise SEQ ID NO: 3.

36. The method of any of one of embodiments 34-35, wherein the CD40L-specific monomer subunits are connected by a linker.

37. The method of any one of embodiments 34-36, wherein at least one CD40L-specific monomer subunit is fused or conjugated to a polyethylene glycol (PEG) directly.

38. The method of any one of embodiments 34-37, wherein at least one CD40L-specific monomer subunit is fused or conjugated to a polyethylene glycol (PEG) via a linker.

39. The method of embodiment 38, wherein the linker comprises a peptide linker.

40. The method of embodiment 39, wherein the linker comprises SEQ ID NO: 7, SEQ ID NO: 8, SEQ ID NO: 9, or SEQ ID NO: 10.

41. The method of any one of embodiments 34-40, wherein at least one CD40L-specific monomer subunit is fused or conjugated to an albumin.

42. The method of embodiment 41, wherein the albumin is human serum albumin (HSA).

43. The method of embodiment 42, wherein the HSA is a variant HSA comprising SEQ ID NO: 4.

44. The method of any one of embodiments 1-42, wherein the Tn3 scaffold comprises SEQ ID NO: 1.

### Embodiment Set 6

1. Use of a Tn3 scaffold for the preparation of a medicament for treating Sjögren's syndrome (SS) in a subject in need thereof, wherein the medicament is formulated for administration at a dose of 1500 mg once every 4 weeks, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject has moderate-to-severe systemic disease activity of ≥5 as determined by European Alliance of Associations for Rheumatology (EULAR) SS Disease Activity Index (ESSDAI).

2. The use of embodiment 1, wherein prior to the once every 4 week dosing, the subject was administered at least 3 loading doses of 1500 mg each.

3. The use of embodiment 2, wherein the at least 3 loading doses are administered at week 0, 2, and 4.

4. Use of a Tn3 scaffold for the preparation of a medicament for treating Sjögren's syndrome (SS) in a subject in need thereof, wherein the medicament is formulated for administration at a dose of 3000 mg once every 12 weeks, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject has moderate-to-severe systemic disease activity of ≥5 as determined by European Alliance of Associations for Rheumatology (EULAR) SS Disease Activity Index (ESSDAI).

5. The use of embodiment 4, wherein prior to the once every 12 week dosing, the subject was administered at least 3 loading doses of 3000 mg each.

6. The use of embodiment 5, wherein the at least 3 loading doses are administered at week 0, 4, and 12.

7. Use of a Tn3 scaffold for the preparation of a medicament for treating Sjögren's syndrome (SS) in a subject in need thereof, wherein the medicament is formulated for administration at a dose of 1500 mg once every 4 weeks, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject has moderate-to-severe symptom state defined by ESSPRI score of ≥5, with a low systemic disease activity defined by ESSDAI score of <5, and wherein a Diary for Assessing Sjögren's Patient Reported Index (DASPRI) score is reduced in the subject following the administration.

8. The use of embodiment 7, wherein prior to the once every 4 week dosing, the subject was administered at least 3 loading doses of 1500 mg each.

9. The use of embodiment 8, wherein the at least 3 loading doses are administered at weeks 0, 2, and 4.

10. Use of a Tn3 scaffold for the preparation of a medicament for treating Sjögren's syndrome (SS) in a subject in need thereof, wherein the medicament is formulated for administration at a dose of 3000 mg once every 12 weeks, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the subject has moderate-to-severe symptom state defined by ESSPRI score≥5, with a low systemic disease activity defined by ESSDAI score<5, and

wherein a Diary for Assessing Sjögren's Patient Reported Index (DASPRI) score is reduced in the subject following the administration.

11. The use of embodiment 10, wherein prior to the once every 12 week dosing, the subject was administered at least 3 loading doses of 3000 mg each.

12. The use of embodiment 11, wherein the at least 3 loading doses are administered at week 0, 4, and 12.

13. The use of any one of embodiments 1-5, wherein the subject has an ESSDAI score from 5-7, 5-10, 5-13, or 10-13 before the administration of the Tn3 scaffold.

14. The use of any one of embodiments 1-6, wherein the administration is effective at reducing the ESSDAI score.

15. The use of embodiment 14, wherein the ESSDAI score is reduced by at least 1, 2, 3, 4, 5, 6, or 7 points.

16. The use of embodiment 15, wherein the ESSDAI score is reduced by at least 6 points.

17. The use of any one of embodiments 15-16, wherein the ESSDAI score is reduced by at least 1 month, 2 months, 3 months, 4 months, 5 months, 6 months, 7 months, 8 months, 9 months, 10 months, 11 months, or 1 year following administration of the first loading dose as compared to a baseline ESSDAI score.

18. The use of embodiment 17, wherein the ESSDAI score is reduced by 11 months following the administration of the first loading dose.

19. The use of any one of embodiments 7-18, wherein salivary gland function in the subject is improved following administration of the Tn3 scaffold by about 3 weeks, 1 month, 2 months, or 4 months following the administration.

20. The use of embodiment 19, wherein salivary gland function is determined by whole stimulated salivary flow.

21. The use of any one of embodiments 9-20, wherein the ESSPRI score is reduced by at least about 1.5, 2, 3, 4, or 5 points following the loading dose administrations.

22. The use of any one of embodiments 7-21, wherein the DASPRI score is reduced by 2 weeks, 4 weeks, 1 month, 2 months, 3 months, 4 months, 5 months, or 6 months following the administration.

23. The use of embodiment 22, wherein the DASPRI score is reduced by at least about 5, 10, 15, or 20 points following the loading dose administrations.

24. The use of any one of embodiments 1-23, wherein the administration of the Tn3 scaffold is effective at reducing a tender and swollen joint count baseline score of the subject following the administration.

25. The use of any one of embodiments 1-24, wherein the administration of the Tn3 scaffold is effective at reducing a Short Form 36 (SF-36) Health Survey baseline score of the subject following the administration.

26. The use of any one of embodiments 1-25, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

27. The use of any one of embodiments 1-26, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

28. The use of any one of embodiments 1-27, wherein the administration of the Tn3 scaffold is effective at reducing a baseline level of a biomarker, and wherein the biomarker is selected from the group consisting of: plasma soluble CD40L, Ki67+CD27+ memory B cell, CD19, CD20, CD27, CD38, CD138, CD11c bright/high B cell, CD3, CD4, CD8, Tfh cell, serum CXCL13, rheumatoid factor, anti-SSA autoantibodies (i.e., anti-Ro autoantibodies), anti-SSB autoantibodies (i.e. anti-La autoantibodies), and combinations thereof.

29. The use of any one of embodiments 1-28, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

30. The use of any one of embodiments 1-29, wherein expression levels of genes, or levels of proteins encoded by the genes, associated with SS are reduced in a blood sample of the subject by week 48 following the administration.

31. The use of any one of embodiments 1-30, wherein levels of B cells selected from the group consisting of: CD27+ memory B cells, marginal zone B cells, plasmablasts, plasma cells, and combinations thereof, are reduced in a blood sample of the subject by week 48 following the administration.

32. The use of any one of embodiments 1-31, wherein the subject is positive for anti-Ro autoantibodies, rheumatoid factor (RF), or both anti-Ro autoantibodies and RF.

33. The use of any one of embodiments 1-32, wherein the Tn3 scaffold is administered intravenously.

34. The use of any one of embodiments 1-33, wherein the Tn3 scaffold comprises two CD40L-specific monomer subunits connected in tandem.

35. The use of embodiment 34, wherein the two CD40L-specific monomer subunits each comprise SEQ ID NO: 3.

36. The use of any of one of embodiments 34-35, wherein the CD40L-specific monomer subunits are connected by a linker.

37. The use of any one of embodiments 34-36, wherein at least one CD40L-specific monomer subunit is fused or conjugated to a polyethylene glycol (PEG) directly.

38. The use of any one of embodiments 34-37, wherein at least one CD40L-specific monomer subunit is fused or conjugated to a polyethylene glycol (PEG) via a linker.

39. The use of embodiment 38, wherein the linker comprises a peptide linker.

40. The use of embodiment 39, wherein the linker comprises SEQ ID NO: 7, SEQ ID NO: 8, SEQ ID NO: 9, or SEQ ID NO: 10.

41. The use of any one of embodiments 34-40, wherein at least one CD40L-specific monomer subunit is fused or conjugated to an albumin.

42. The use of embodiment 41, wherein the albumin is human serum albumin (HSA).

43. The use of embodiment 42, wherein the HSA is a variant HSA comprising SEQ ID NO: 4.

44. The use of any one of embodiments 1-42, wherein the Tn3 scaffold comprises SEQ ID NO: 1.

### EXAMPLES

### Example 1—A Phase 3, Randomized, Double-blind, Placebo-controlled Study to Evaluate the Efficacy and Safety of Dazodalibep in Participants with Sjögren's Syndrome with Moderate-to-severe Systemic Disease Activity

A phase 3, randomized double-blind, placebo-controlled study to evaluate the efficacy and safety of Dazodalibep in participants with Sjögren's Syndrome (SS) with moderate-to-severe systemic disease activity is disclosed herein.

### Objectives and Endpoints:

Study objectives and endpoints are presented in Table 2.

TABLE 2
Exemplary study objectives and endpoints

| | |
|---|---|
| Objectives | Endpoints |

Primary

| | |
|---|---|
| To evaluate the effect of dazodalibep on systemic | Change from baseline in ESSDAI score at |
| manifestations of SS in participants with moderate- | Week 48 |
| to-severe systemic disease activity | |

Secondary

| | |
|---|---|
| To evaluate the effect of dazodalibep on measures of | Proportion of participants achieving |
| systemic activity and PROs in participants with SS | ESSDAI response, defined as a decrease of |
| | at least 5 points from baseline in the |
| | ESSDAI at Week 48 without premature |
| | discontinuation from treatment and without |
| | receiving rescue or potentially confounding |
| | therapy |
| | Change from baseline in ESSPRI dryness |
| | domain score at Week 48 |
| | Change from baseline in tender and swollen |
| | joint counts at Week 48 |
| | Change from baseline in SF-36 PCS score at |
| | Week 48 |

Exploratory Objective(s) and Exploratory Endpoint(s)

| | |
|---|---|
| To evaluate biomarkers of inflammation and | Change from baseline in levels of markers |
| autoantibodies in participants with SS upon | of inflammation (immunoglobulins, β-2 |
| treatment with dazodalibep | microglobulin, and C-reactive protein |
| | [CRP]) |
| To evaluate dazodalibep target engagement (TE) | Change from baseline in levels of plasma |
| | soluble CD40L |
| To evaluate the PD of dazodalibep | Change from baseline in rheumatoid factor |
| | (RF). |
| | Change from baseline in levels of |
| | biomarkers including but not limited to |
| | peripheral blood flow cytometry for changes |
| | in B-cell subsets, serum CXCL 13. |
| To evaluate the effects of dazodalibep on systemic | Proportion of participants achieving |
| disease activity as well as in joints and salivary and | ESSDAI[3] or ESSDAI[4] response |
| lacrimal function in participants with SS. | Proportion of participants achieving |
| | ESSDAI < 5 |
| | Change in Physician Global Impression of |
| | Severity (MDGIS) |
| | Change from baseline in salivary gland |
| | ultrasound imaging (subset of participants) |
| | Change from baseline in joint ultrasound |
| | imaging (subset of participants) |
| To evaluate changes in key subjective complaints | Change from baseline in ESSPRI total score |
| associated with SS. | Change from baseline in ESSPRI pain |
| | domain score |
| | Change from baseline in ESSPRI fatigue |
| | domain score |
| | Proportion of participants achieving ESSPRI |
| | response, defined as a decrease of at least |
| | 1.5 points from baseline in the ESSPRI, |
| | without premature discontinuation from |
| | treatment and without receiving rescue or |
| | potentially confounding therapy. |
| | Change from baseline in FACIT-Fatigue |
| | score |
| | Change from baseline in VAS oral dryness |
| | Change from baseline in VAS ocular |
| | dryness |
| | Change from baseline in VAS vaginal |
| | dryness |
| | Change from baseline in Total Stimulated |
| | Salivary Flow |
| | Change from baseline in Patient Global |
| | Impression of Severity (PGIS) |
| | Patient Global Impression of Change |
| | (PGIC) |
| | Change from baseline in Female Sexual |
| | Function Index (FSFI) (where appropriate) |
| | Change from baseline in Clinical EULAR |
| | Sjogren'sSyndrome Disease Activity Index |
| | (ClinESSDAI) |
| To assess effects of dazodalibep on blood gene | Change from baseline in global gene |
| expression | expression by RNA PAXgene assay |
| To characterize exploratory biomarkers of disease | Change from baseline in levels of plasma, |
| and response to treatment | serum, saliva, and whole blood exploratory |
| | biomarkers |
| To characterize the PK of dazodalibep in participants | Plasma concentration of dazodalibep |
| with SS | |
| To assess the immunogenicity of multiple doses of | Proportion of participants who develop anti- |
| dazodalibep in participants with SS | drug antibodies (ADAs) over time |

### Study Design

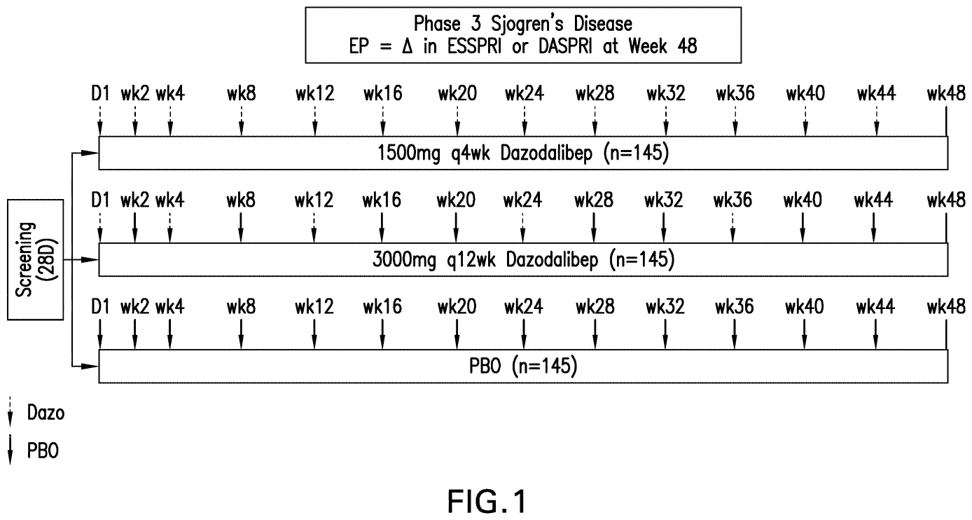

This study is a Phase 3, randomized, double-blind, placebo-controlled study to evaluate the efficacy and safety of Dazodalibep in participants aged ≥18 diagnosed with Sjögren's syndrome (SS) with moderate-to-severe disease activity as defined by EULAR Sjögren's syndrome disease activity index (ESSDAI) score≥5. The study design is provided in FIG. 1.

Individuals will undergo a screening period (see Table 3) of up to 28 days followed by randomization and treatment through the Week 44 visit. Approximately 510 participants will be randomized (1:1:1) to Dazodalibep Dose 1, Dazodalibep Dose 2, or placebo as follows:
Dazodalibep Dose 1:1500 mg intravenously (IV) at Weeks 0, 2, and 4 then once every 4 weeks (Q4W) (13 total doses: 13 doses of dazodalibep) (n=170)
Dazodalibep Dose 2:3000 mg IV at Weeks 0, 4, and 12 then once every 12 weeks (Q12W) (13 total doses: 5 doses of dazodalibep; 8 doses of placebo) (n=170)
Placebo (13 total doses: 13 doses of placebo) (n=170)

Participants will receive randomized treatment dazodalibep or placebo) through Week 44. The primary endpoint visit will be at Week 48. At the Week 44 visit, eligible participants may provide written informed consent to screen for an open-label extension (OLE) study for receipt of open-label dosing with dazodalibep. Participants ineligible for or not wishing to enroll in the OLE extension will have a final study visit at Week 56 after the last dose of investigational product (IP). The expected full duration of each individual's participation in this study, including screening, is up to 420 days.

Eligible participants interested in participating in the OLE will complete participation in this Phase 3 study at the Week 48 visit, after completion of all assessments for the visit. These Week 48 visit values will serve as baseline (Day 1) for the OLE study, and Dose 1 of open-label dazodalibep will be administered at or at approximately the Week 48 visit, their final visit in this Phase 3 study. All participants, including those who enroll in the OLE, will remain blinded to their treatment assignment in Phase 3 until the Phase 3 study is complete.

The primary objective of this study is to evaluate the effect of dazodalibep on systemic manifestations of SS in participants with moderate-to-severe systemic disease activity. This will be assessed by evaluating the change from baseline in ESSDAI score at Week 48.

TABLE 3
Exemplary schedule of screening procedures

| Study Period | Screening |
|---|---|
| Visit Number | V1 |
| Procedure/Study Day | Day -28 to Day -1 |
| ESSPRI | X |
| PGIS | X |
| Medical history | X |
| Assessment of prior and concomitant medications | X |
| 2016 ACR/EULAR Criteria for classification of Sjogren'ssyndrome | X |
| Vital signs | X |
| Complete physical examination (including weight and height) | X |
| 28-joint count for swelling and tenderness | X |
| ESSDAI | X |
| Whole stimulated salivary flow | X |
| Virology: Hepatitis B, Hepatitis C; HIV-1; HIV-2 | X |
| TB test (IGRA) | X |
| Safety Lab Tests: Serum Chemistry, Clinical Chemistry, Hematology, | X |
| Urinalysis, Urine Protein/Creatinine Ratio | |
| Coagulation panel (prothrombin time, INR, PTT) | X |
| Autoantibody panel (anti-SSA [Ro], anti-SSB [La], ANA) | X |
| Rheumatoid factor (RF) | X |
| ESSDAI-required blood tests (serum C3, C4, IgG, serum and urine immunofixation, | X |
| cryoglobulins, serum free light chains) ^{a} | |
| Pregnancy test (serum β-hCG; females of childbearing potential only) | X |
| FSH where appropriate to confirm menopausal state | X |
| Electrocardiogram (if performed after phlebotomy, separate by 10 minutes) | X |
| Chest X-ray ^{b} | X |
| Preliminary determination of eligibility | X |

ACR = American College of Rheumatology;
ANA = antinuclear antibodies;
APL = antiphospholipid;
β-hCG = beta-human chorionic gonadotropin;
C = complement;
ESSDAI = EULAR Sjogren'sSyndrome Disease Activity Index;
ESSPRI = EULAR Sjogren'sSyndrome Patient Reported Index;
EULAR = European Alliance of Associations for Rheumatology;
HIV = human immunodeficiency virus;
Ig = immunoglobulin; IGRA = Interferon Gamma Release Assay;
INR = international normalized ratio;
PGIS = Patient Global Impression of Severity;
PTT = partial thromboplastin time;
FSH = follicle-stimulating hormone;
TB = tuberculosis;
V = visit.
^{a} If cryoglobulin results are delayed such that an individual could not complete screening within 28 days, and if the ESSDAI score is at least 5 without a cryoglobulin result, the individual can be considered to be eligible.
^{b} Chest X-ray not required during screening if performed within 12 weeks prior to screening visit and result is available.

### Study Population

### Inclusion Criteria

To be included in this study, individuals may satisfy all the following criteria:
1. Adults, ≥18 years at time of informed consent.
2. Diagnosed with SS by meeting the 2016 American College of Rheumatology (ACR)/EULAR Classification Criteria.
3. Have an ESSDAI score of ≥5 at screening.
4. Positive for either anti-Ro autoantibodies or rheumatoid factor (RF), or both at screening.
5. Females of childbearing potential who are sexually active with a non-sterilized male partner must use a highly effective method of contraception from signing the informed consent form (ICF) and must agree to continue using such precautions through the end of the study; cessation of contraception after this point should be discussed with a responsible physician. Highly effective methods of contraception include:
   a. Combined (estrogen and progestogen containing) hormonal contraception associated with inhibition of ovulation:
      i. Oral
      ii. Intravaginal
      iii. Transdermal
      iv. Injectable
   b. Progestogen-only hormonal contraception associated with inhibition of ovulation:
      i. Oral
      ii. Injectable
      iii. Implantable
   c. Intrauterine device
   d. Intrauterine hormone-releasing system
   e. Bilateral tubal occlusion
   f. Azoospermic partner (vasectomized or due to a medical cause)
      i. Azoospermia is a highly effective contraceptive method provided that the partner is the sole sexual partner of the woman of childbearing potential and the absence of sperm has been confirmed. If not, an additional highly effective method of contraception should be used. Spermatogenesis cycle is approximately 90 days.
   g. Sexual abstinence
      i. Sexual abstinence is considered a highly effective method only if it is the preferred and usual lifestyle of the participant and the participant agrees to refrain from heterosexual intercourse from screening through the end of the study follow-up. Periodic abstinence, the rhythm method, and the withdrawal method are not acceptable methods of contraception. A recommendation that the female partners (of childbearing potential) of male study participants should use a highly effective method of contraception other than a barrier method should be made.
         1. Females of childbearing potential are defined as those who are not surgically sterile (surgical sterilization includes bilateral tubal ligation, bilateral oophorectomy, or hysterectomy) or those who are not postmenopausal (defined as 12 months with no menses without an alternative medical cause).
         2. Vasectomized partner is a highly effective birth control method provided that the partner is the sole sexual partner of the woman of childbearing potential trial participant and that the vasectomized partner has received medical assessment of the surgical success.
6. Non-sterilized male participants who are sexually active with a female partner of childbearing potential must use a male condom with spermicide and refrain from donating fresh unwashed semen from Day 1 through the end of the study. His female partner should also be advised of the benefit to use a highly effective method of contraception, as a condom may break or leak.
7. Vaccinated against severe acute respiratory syndrome coronavirus 2 (SARS-COV-2) according to current local authority guidelines, if any, at least 2 weeks prior to screening unless the participant refuses vaccination. Initial or subsequent COVID-19 vaccine administration is permitted during the study as long as it is not administered during the screening period or within a week after Dose 1; if vaccine is to be administered during this window, screening should be delayed to complete vaccination.
8. Meets all of the following tuberculosis (TB) criteria:
   a. No history of latent or active TB prior to screening, except for latent TB with documented completion of locally appropriate treatment.
   b. No signs or symptoms suggestive of active TB from medical history or physical examination.
   c. No recent (≤12 weeks of screening) close contact with a person with active TB (close contact is defined as ≥4 hours/week or living in the same household OR in a house where a person with active TB is a frequent visitor).
   d. Negative Interferon Gamma Release Assay (IGRA) test result for TB at screen unless previously treated as per Inclusion Criterion 8 (a). Subjects with an indeterminate test result can repeat the test, but if the repeat test is also indeterminate, they are excluded.
   e. A chest radiograph (obtained during the screening period or any time within 12 weeks prior to screening) with no evidence of current active TB or other infection, or prior TB, malignancy, or clinically significant abnormalities suggesting an active process (unless due to SS).

### Exclusion Criteria

If an individual meets any of the following criteria, he or she is ineligible for this study:
1. Individuals with medical history of confirmed deep venous thrombosis, pulmonary embolism, or arterial thromboembolism within 2 years of screening.
2. History or presence of concomitant polymyositis or dermatomyositis or systemic sclerosis.
3. Active malignancy or history of malignancy within the last 5 years, except as follows:
   a. In situ carcinoma of the cervix treated with apparent success with curative therapy ≥12 months prior to screening; or,
   b. Cutaneous basal cell carcinoma following presumed curative therapy.
4. Individuals who are pregnant or lactating or planning to become pregnant during the study.
5. Individuals who have a positive test for, or have been treated for, hepatitis B or human immunodeficiency virus (HIV) infection. A positive test for hepatitis B infection at screening is defined as: (1) positive for hepatitis B surface antigen; or (2) positive for hepatitis B core antibody. Individuals with a positive test for or a history of treatment for hepatitis C are excluded unless they have a documented sustained viral response to antiviral drugs approved for the treatment of hepatitis C, defined as an undetectable viral level of hepatitis C RNA at least 24 weeks following completion of therapy. Individuals with advanced fibrosis or cirrhosis due to hepatitis C should not be enrolled.
6. Individuals with a positive test for SARS-COV-2 on the day of randomization or symptoms suggestive of SARS-COV-2 at randomization or significant exposure to coronavirus disease 2019 (COVID-19) within 10 days prior to randomization. Individuals with COVID-19 or COVID-19 exposure can delay randomization for 10 days and randomize once recovered; otherwise, they will need to rescreen.
7. Individuals with:
   a. A history of more than one episode of herpes zoster and/or any opportunistic infection in the last 12 months (see Table 4), with the exception of non-invasive herpes simplex at any site, oral candidiasis, vaginal candidiasis, or cutaneous fungal infections, which are permitted within the prior 12 months unless of unusual severity.
   b. Active infections requiring systemic treatment at the time of screening or through randomization, or history of more than 2 infections requiring IV antibiotics within 12 months prior to screening.
8. Individuals with known history of severe allergy or reaction to any component of the IP formulation or to any other biologic therapy.
9. Individuals with any severe cardiovascular, respiratory, endocrine, gastrointestinal, hematological, neurological, psychiatric, or systemic disorder or any other condition that, in the opinion of the Investigator, would place the individual at unacceptable risk of complications, interfere with evaluation of the IP.
10. Individuals who are unable or unwilling to comply with protocol requirements (e.g., active drug or alcohol abuse or for other reasons).
11. Individuals who have received a live (attenuated) vaccine within the 4 weeks prior to randomization or plan to receive a live vaccine during their participation in the study. Non-live vaccines are permitted during the study (see Inclusion Criterion 7 for COVID-19 vaccines); however, for subjects who plan to receive a vaccine within a month after dose 1, completing vaccination prior to starting dosing should be considered.
12. Last administration of experimental or investigational biologic or oral agents (other than those listed in Exclusion Criterion 16)<6 months prior to screening.
13. Individuals who have had previous treatment with any biologic B-cell-depleting therapy (e.g., rituximab, ocrelizumab, inebilizumab, ofatumumab, or ianalumab) within 12 months or other B-cell-targeting therapy (e.g., belimumab)<3 months prior to screening.
14. Injectable corticosteroids (including intra-articular [IA] or intra-muscular [IM]) or treatment with >10 mg/day dose of oral prednisone or equivalent within 6 weeks prior to randomization. Concomitant treatment with oral corticosteroids≤10 mg/day prednisone or equivalent for underlying SS, rheumatoid arthritis (RA), or systemic lupus erythematosus (SLE) is permitted provided that the dose is stable for ≥2 weeks prior to screening through randomization (Day 1) and is expected to remain stable for the duration of the treatment period. Inhaled, intranasal, or topical corticosteroids are allowed provided doses are expected to be stable during the study.
15. Individuals treated with systemic corticosteroids for indications other than SS, RA, and SLE for more than a total of 2 weeks within 6 months prior to screening.
16. Use of the following medications:
   a. Antimalarials (e.g., chloroquine, hydroxychloroquine, quinacrine) if they have been initiated or if the dose has changed within 8 weeks prior to screening.
   b. Methotrexate (MTX) if the dose is >25 mg/week or if there is any change or initiation of a new dose within 4 weeks prior to screening.
   c. Azathioprine if the dose is >150 mg/day or if there is any change in dose or initiation of new dose within 4 weeks prior to screening.
   d. Leflunomide if the dose is >20 mg/day or if there is any change or initiation of new dose within 4 weeks prior to screening.
   e. Mycophenolate mofetil (MMF) if the dose is >2 g/day or if there is any change to or initiation of a new dose within 4 weeks prior to screening.
   f. Any other disease-modifying antirheumatic drug (DMARD), immunosuppressant, or antiproliferative agents if last dose was taken within:
      i. 4 weeks prior to screening; OR
      ii. Drug-specific 5 half-lives elimination period (if longer than 4 weeks).
   g. Any medication that, in the opinion of the Investigator, would interfere with evaluation of the IP or interpretation of study results.
   h. Any increase or initiation of new doses of cyclosporine eye drops (Restasis®) or lifitegrast (Xiidra®) eyedrops within 2 weeks prior to screening.
   i. If being taken, adjustments to the above medications are not permitted during the screening period, and medications are expected to remain stable for the entire study duration.
17. Individuals who have received previous treatment with anti-CD40L compounds at any time before screening.
18. Individuals with blood tests, at screening, of any of the following:
   a. Aspartate aminotransferase (AST)>2×upper limit of normal (ULN)
   b. Alanine aminotransferase (ALT)>2×ULN
   c. Total bilirubin (TBL)>2×ULN, unless Gilbert's syndrome is documented in the
   medical history
   d. Hemoglobin<75 g/L
   e. Neutrophils<0.8×10⁹/L
   f. Lymphocytes<0.8×10⁹/L
   g. Platelets<100×10⁹/L
   h. International normalized ratio (INR) for prothrombin time>1.3×ULN

TABLE 4
Exemplary infections that meet exclusion criteria

| Definite ^{a,b} Opportunistic Infection | Probable ^{c} Opportunistic Infection |
|---|---|
| Pneumocystis jirovecii | Paracoccidioides infections |
| BK virus disease, including polyomavirus-associated nephropathy | Penicillium marneffei (talaromyces) |
| CMV disease | Sporothrix schenckii |
| Post-transplant lymphoproliferative disorder (EBV) | Cryptosporidium species |
| Progressive multifocal leukoencephalopathy | (chronic disease only) |
| Bartonellosis (disseminated disease only) | Microsporidiosis |
| Blastomycosis | Leishmaniasis (visceral only) |
| Toxoplasmosis | Trypanosoma cruzi infection |
| Coccidioidomycosis | (Chagas's disease) (disseminated disease only) |
| Histoplasmosis | Campylobacteriosis (invasive disease only) |
| Aspergillosis (invasive disease only) | Shigellosis (invasive disease only) |
| Candidiasis (invasive disease or pharyngeal) | Vibriosis |
| Cryptococcosis | (invasive disease due to Vibrio vulnificus) |
| Other invasive fungi: Mucormycosis (zygomycosis) | HCV progression |
| (Rhizopus, Mucor, and Lichtheimia), | |
| Scedosporium/Pseudallescheria boydii, Fusarium) | |
| Legionellosis | |
| Listeria monocytogenes (invasive disease only) | |
| TB | |
| Nocardiosis | |
| Non-tuberculous mycobacterium disease | |
| Salmonellosis (invasive disease only) | |
| HBV reactivation | |
| Herpes simplex (invasive disease only) | |
| Herpes zoster (any form) | |
| Strongyloides | |
| (hyperinfection syndrome and disseminated forms only) | |

CMV = cytomegalovirus;
EBV = Epstein-Barr virus;
HBV = hepatitis B virus;
HCV = hepatitis C virus;
TB = tuberculosis. Source: Winthrop et al 2015.
^{a} Generally does not occur in the absence of immunosuppression, but whose presence indicates a potential or likely alteration in host immunity.
^{b} Can occur in patients without recognized forms of immunosuppression, but whose presence indicates a potential or likely alteration in host immunity. Published data are currently lacking, but expert opinion believes that risk is likely elevated in the setting of biologic therapy.

### Lifestyle Consideration

Meat and dietary restrictions: There are no dietary or fasting restrictions during the study.

Activity: There are no activity restrictions for this study.

### Criteria for Temporarily Delaying Randomization

Individuals with COVID-19 or COVID-19 exposure can delay randomization for 10 days and randomize once recovered; otherwise, they may need to rescreen (See Exclusion Criterion 11).

### Study Interventions and Concomitant Therapy

### Investigational Product Administered

Administration of IP will be performed by a blinded administrator. Preparation of IP will be performed by an uninvolved, unblinded pharmacist/IP manager or study-site staff member. The prepared IP must be covered with a bag prior to providing it to the blinded administrator.

Table 5 includes a description of the IPs used in this study, their dose formulation, unit dose strength, dosage level, route of administration, use, sourcing, and packaging.

TABLE 5
Exemplary descriptions of the IPs used in this study

| Intervention | | | |
|---|---|---|---|
| Name | Dazodalibep 1500 mg | Dazodalibep 3000 mg | Placebo |
| Dose | 6R vial with nominal fill | 6R vial with nominal fill volume of | 6R vial with nominal fill |
| Formulation | volume of 5 mL formulated | 5 mL formulated at 100 mg/mL of | volume of 5 mL of 10 |
| | at 100 mg/ml of | dazodalibep in 10 mM sodium | mM sodium phosphate |
| | dazodalibep in 10 mM | phosphate monobasic/dibasic, 250 | monobasic/dibasic, 250 |
| | sodium phosphate | mM sucrose, and 0.02% | mM sucrose, and 0.02% |
| | monobasic/dibasic, 250 | (weight/volume) poloxamer 188, | (weight/volume) |
| | mM sucrose, and 0.02% | pH 7.4. | poloxamer 188, pH 7.4. |
| | (weight/volume) poloxamer | | |
| | 188, pH 7.4. | | |
| Type | Drug | Drug | Placebo |
| Unit Dose | 100 mg/mL dazodalibep | 100 mg/mL dazodalibep nominal | Not applicable |
| Strength(s) | nominal | | |
| Dosage Level(s) | 1500 mg | 3000 mg | Not applicable |
| Route of | IV infusion | IV infusion | IV infusion |
| Administration | | | |

### Blinding and Masking

### Blinding

Participants will be randomly assigned in a 1:1:1 ratio to study arms (see Table 6).

TABLE 6
Exemplary study arm(s)

| Arm Title | 1500 mg Q4W dazodalibep | 3000 mg Q12W dazodalibep | Placebo |
|---|---|---|---|
| Arm Type | Experimental | Experimental/placebo | Placebo |
| Arm | Participants will receive | Participants will receive | Participants will |
| | dazodalibep 1500 mg IV at | dazodalibep 3000 mg IV at Weeks | receive placebo IV |
| | | 0, 4, and 12 then Q12W for a total | |
| Description | Weeks 0, 2, and 4 then Q4W | of 13 total doses (5 doses of | at Weeks 0, 2, and |
| | for a total of 13 total doses | dazodalibep and 8 doses of placebo) | 4, then Q4W for a |
| | | | total of 13 total |
| | | | doses |
| Associated | Dazodalibep 1500 mg | Dazodalibep 3000 mg, placebo | Placebo |
| Intervention | | | |
| Labels | | | |

IV = intravenous;
Q4W = once every 4 weeks;
Q12W = once every 12 weeks.

Continued Access to IP after the End of the Study

Participants will receive randomized treatment (dazodalibep or placebo) through Week 44. The primary endpoint visit will be at Week 48. At the Week 44 visit, eligible participants may screen for an OLE study for receipt of open-label dosing with dazodalibep starting after the completion of this study at Week 48.

The schedule of study assessments during the treatment period is provided in Table 7.

### Prior and Concomitant Therapy

Any medication or vaccine (including over-the-counter or prescription medicines, recreational drugs, vitamins, and/or herbal supplements) or other specific categories of interest that the participant has received in the previous 12 months, is receiving at the time of enrollment, or receives during the study must be recorded along with:
1. Name and indication
2. Reason for use
3. Dates of administration including start and end dates
4. Dosage information including dose and frequency

### Permitted Concomitant Therapy

All concomitant medications (from Visit 1) taken while the participant is in the study will be recorded. All medications needed for the health of the participant are permitted. Prohibited medications are those that may require discontinuation of IP. Participants must continue to study completion even if prohibited medications were taken.

Permitted medications are any medications required that are not specifically prohibited by the protocol during the clinical study (i.e., from Visit 1 to the end of the safety follow-up period).

### Prohibited Concomitant Medications

Prohibited medications are considered to be potentially confounding, unsafe, or both. Prohibited medications are:
Medications that should be avoided, if possible, because they could confound interpretation of the data.
Medications that may be potentially unsafe to co-administer with the IP and will lead to discontinuation of IP.

Participants who discontinue IP may remain in the study and complete all study visits and assessments except those assessments directly related to dosing. For analysis, the following drugs are considered to be prohibited medications. These may or may not require discontinuation of IP.

### Prohibited Medications

Any rescue medicine (see “Rescue Medicines” below).
Use of an increased dose or initiation of oral corticosteroids for general medical purposes (e.g., such as treatment of hives, poison ivy, asthma, etc.) other than use as rescue medication is prohibited in the following situations:
   Any use of dexamethasone (or other long-acting corticosteroid).
   Use of new or increased dose of baseline corticosteroid for longer than 21 cumulative days.
   Any new or increased dose of baseline corticosteroids administered after the Week 36 visit.
   Any dose that exceeds 50 mg prednisone equivalents per day.
Initiation of intravenous (IV) or intramuscular (IM) corticosteroids for general medical purposes (i.e., such as treatment of hives, poison ivy, asthma, etc.) other than use as a rescue medication is prohibited in the following situations:
   Any use of dexamethasone (or other long-acting corticosteroid).
   Use of ≥1 g of methylprednisolone (or equivalent) cumulative exposure.
   Any use of IV or IM corticosteroids after the Week 36 visit.

The following medications or interventions may require discontinuation of IP; however, participants should continue to be followed in accordance with the Table 7 except for activities solely related to infusion unless they withdraw consent for continued participation.
Cytokine or interleukin (e.g., TNF-α, IL-6, IL-12/23, IL-17, IFN, etc.) blocking therapies, complement inhibitors, abatacept, Janus kinase (JAK) inhibitors, rituximab, ocrelizumab, inebilizumab, cyclophosphamide, or tacrolimus.
IV or IM corticosteroids>1 g of methylprednisolone (or equivalent) cumulative exposure.
IV Immunoglobulin (Ig; targeted Ig for treatment or prevention of an infectious disease, such as COVID-19, is permitted).
Bone marrow, stem cell, or solid organ transplant.
Plasmapheresis, plasma exchange.
Investigational agents.
Any concomitant medication or medication dose for treatment of SS that was prohibited in protocol exclusion criteria may or may not determine continuation of IP.

### Rescue Medicines

Rescue medications are new or an increase of baseline medications intended to treat SS or any associated underlying rheumatologic condition. These include:
Any initiation or increase of baseline azathioprine, MTX, leflunomide, mycophenolate mofetil (MMF), or antimalarial (e.g., chloroquine, hydroxychloroquine, quinacrine, etc.)
Initiation of any biologic DMARD, conventional DMARD not listed, or similar systemic immune-modulating or immunosuppressive therapy
Any initiation or increase of baseline oral corticosteroid dose
Initiation of IV, IM, or IA/tendon sheath/bursal injection
Use of corticosteroids or adrenocorticotropic hormone

Use of rescue medications may result in discontinuation of IP.

### Discontinuation of IP

It may be necessary for a participant to permanently discontinue IP. Discontinuation of IP does not require withdrawal from the study, and participants who discontinue IP may be followed until study completion in accordance with the schedule of assessments (Table 7).

Note that missing a final dose of IP does not result in treatment discontinuation.

### Study-Specific Investigational Product Discontinuation Criteria

The IP may be permanently discontinued for the reasons described below.
Receipt of prohibited medications that require discontinuation of IP
Pregnancy, lactation, or a decision to become pregnant
Any of the following liver function abnormalities:
   ALT or AST>8×ULN;
   ALT or AST>5×ULN for more than 2 weeks.
   Liver injury meeting the definition of Hy's law related to dazodalibep or with unknown etiology.
Anaphylaxis or a Grade 4 hypersensitivity reaction attributed to Dazodalibep.
Grade 4 infusion-related reaction. An infusion may be restarted or a participant may be dosed at the next dosing visit after a Grade 1, 2, or 3 infusion-related reaction that did not require hospitalization as long as the participant was not already receiving premedication and can safely be dosed with premedication that does not require glucocorticoids that would require termination of dosing. Participants with recurrent Grade 1 or 2 infusion-related reactions can be managed with non-glucocorticoid premedication.
Any opportunistic infection (Table 4); events of herpes simplex, mucosal/cutaneous fungal infection, or a single event of herpes zoster, unless of unusual severity, would not be considered to be opportunistic.
Malignancy

Participants who discontinue IP early must continue with scheduled visits, with the exception of dosing, until the completion of the study (Week 56 safety follow-up) unless the participant withdraws from the study. Participants who plan to withdraw consent for study participation but agree to hold an additional visit prior to withdrawal of consent should have a final visit that includes all procedures from the Week 48 visit.

A participant who is unwilling to come to the clinic for the visit may have substitution of a telephone visit for the collection of safety information as the participant will permit.

TABLE 7
Exemplary schedule of assessments

| | Visit No. (V) | | | | | | | | | | | | | | |
|---|---|---|---|---|---|---|---|---|---|---|---|---|---|---|---|
| | 2 | 3 | 4 | 5 | 6 | 7 | 8 | 9 | 10 | 11 | 12 | 13 | 14 | 15^{a} | 16^{b} |
| | Week | | | | | | | | | | | | | | |
| | 0 | 2 | 4 | 8 | 12 | 16 | 20 | 24 | 28 | 32 | 36 | 40 | 44 | 48 | 56 |
| | Study day | | | | | | | | | | | | | | |
| | | 15 − 3 d | 29 ± | 57 ± | 85 ± | 113 ± | 141 ± | 169 ± | 197 ± | 225 ± | 253 ± | 281 ± | 309 ± | 337 ± | 393 ± |
| Procedure | 1 | to +1 d | 4 d | 7 d | 7 d | 7 d | 7 d | 7 d | 7 d | 7 d | 7 d | 7 d | 7 d | 7 d | 7 d |
| ESSPRI | X | | X | X | X | X | X | X | X | X | X | X | X | X | |
| SF-36v2 | X | | X | X | X | X | X | X | X | X | X | X | X | X | |
| FACIT-Fatigue | X | | X | X | X | X | X | X | X | X | X | X | X | X | |
| VAS oral, ocular, | X | | X | X | X | X | X | X | X | X | X | X | X | X | |
| and vaginal | | | | | | | | | | | | | | | |
| PGIS | X | | X | X | X | X | X | X | X | X | X | X | X | X | |
| PGIC | | X | X | X | X | X | X | X | X | X | X | X | X | X | |
| FSFI | X | | X | X | X | X | X | X | X | X | X | X | X | X | |
| SARS-CoV-2 rapid | X | | | | | | | | | | | | | | |
| test ^{c} | | | | | | | | | | | | | | | |
| Concomitant | X | X | X | X | X | X | X | X | X | X | X | X | X | X | X |
| medications | | | | | | | | | | | | | | | |
| Vital signs (BP, | X | X | X | X | X | X | X | X | X | X | X | X | X | X | X |
| HR, RR, and | | | | | | | | | | | | | | | |
| temperature) | | | | | | | | | | | | | | | |
| Weight | X | | X | X | X | X | X | X | X | X | X | X | X | X | |
| Full physical exam | X | | | | | | | X | | | | | | X | |
| Symptom-driven | | X | X | X | X | X | X | | X | X | X | X | X | | X |
| physical exam ^{d} | | | | | | | | | | | | | | | |
| Physical exam for | X | | X | X | X | X | X | X | X | X | X | X | X | X | |
| ESSDAI score | | | | | | | | | | | | | | | |
| 28-joint assessment | X | | X | X | X | X | X | X | X | X | X | X | X | X | |
| (TJC and SJC) | | | | | | | | | | | | | | | |
| Stimulated salivary | X | | | | X | | | X | | | X | | | X | |
| flow measurement | | | | | | | | | | | | | | | |
| ESSDAI | X | | X | X | X | X | X | X | X | X | X | X | X | X | |
| Physician Global | X | | X | X | X | X | X | X | X | X | X | X | X | X | |
| Impression of | | | | | | | | | | | | | | | |
| Severity | | | | | | | | | | | | | | | |
| Electrocardiogram | X | | | | | | | X | | | | | | X | |
| Safety lab tests ^{e} | X | X | X | X | X | X | X | X | X | X | X | X | X | X | X |
| Urine pregnancy | X | X | X | X | X | X | X | X | X | X | X | X | X | X | X |
| test in women of | | | | | | | | | | | | | | | |
| childbearing | | | | | | | | | | | | | | | |
| potential | | | | | | | | | | | | | | | |
| Urinalysis | X | X | X | X | X | X | X | X | X | X | X | X | X | X | X |
| Urine | X | | X | X | X | X | X | X | X | X | X | X | X | X | |
| immunofixation | | | | | | | | | | | | | | | |
| and urine | | | | | | | | | | | | | | | |
| protein/creatinine | | | | | | | | | | | | | | | |
| ratio | | | | | | | | | | | | | | | |
| Coagulation tests | X | | | | X | | | X | | | X | | | X | X |
| (prothrombin | | | | | | | | | | | | | | | |
| time, PTT, INR) | | | | | | | | | | | | | | | |
| Autoantibody | | | | | X | | | X | | | X | | | X | |
| panel ^{f} | | | | | | | | | | | | | | | |
| Rheumatoid | X | | X | X | X | X | X | X | X | X | X | X | X | X | |
| factor | | | | | | | | | | | | | | | |
| Inflammatory | X | X | X | X | X | X | X | X | X | X | X | X | X | X | |
| markers ^{g} | | | | | | | | | | | | | | | |
| Serum C3, C4, | X | | X | X | X | X | X | X | X | X | X | X | X | X | |
| free light chains, | | | | | | | | | | | | | | | |
| serum, and | | | | | | | | | | | | | | | |
| cryoglobulins | | | | | | | | | | | | | | | |
| Exploratory flow | X | X | X | | X | | | X | | | | | | X | |
| cytometry sample | | | | | | | | | | | | | | | |
| (whole blood) ^{h} | | | | | | | | | | | | | | | |
| Exploratory | X | X | X | | X | | | X | | | X | | | X | |
| biomarker sample | | | | | | | | | | | | | | | |
| (serum) ^{h} | | | | | | | | | | | | | | | |
| Exploratory | X | X | X | | X | | | X | | | | | | X | |
| biomarker sample | | | | | | | | | | | | | | | |
| (plasma) ^{h} | | | | | | | | | | | | | | | |
| PBMC collection | X | X | X | | X | | | X | | | | | | X | |
| (whole blood) | | | | | | | | | | | | | | | |
| Saliva for protein | X | | | | X | | | X | | | | | | X | |
| biomarkers | | | | | | | | | | | | | | | |
| ADA (plasma) | X | | | | X | | | X | | | X | | | | X |
| RNA PAXgene | X | X | X | | X | | | X | | | | | | X | |
| (whole blood) | | | | | | | | | | | | | | | |
| Pharmacokinetics | X | | | | X | | | X | | | X | | | X | X |
| (plasma) | | | | | | | | | | | | | | | |
| Ultrasound | X | | | | | | | | X | | | | | X | |
| imaging of joints | | | | | | | | | | | | | | | |
| Salivary gland | X | | | | | | | | X | | | | | X | |
| ultrasound | | | | | | | | | | | | | | | |
| imaging | | | | | | | | | | | | | | | |
| Randomization | X | | | | | | | | | | | | | | |
| Administration of | X | X | X | X | X | X | X | X | X | X | X | X | X | X | |
| IP | | | | | | | | | | | | | | | |

ADA = anti-drug antibodies; AE = adverse event; BP = blood pressure; C = complement; d = day; EDV = early discontinuation visit; EULAR = European Alliance of Associations for Rheumatology; ESSDAI = EULAR Sjögren's Syndrome Disease Activity Index; ESSPRI = EULAR Sjögren's Syndrome Patient Reported Index; FACIT-Fatigue = Functional Assessment of Chronic Illness Therapy - Fatigue; FSFI = Female Sexual Functioning Index; HR = heart rate; Ig = immunoglobulin; INR = international normalized ratio; IP = investigational product; OLE = open-label extension; PBMC = peripheral blood mononuclear cells; PGIC = Patient Global Impression of Change; PGIS = Patient Global Impression of Severity; PTT = partial thromboplastin time; RR = respiratory rate; SAE = serious adverse event; SARS-CoV-2 = severe acute respiratory syndrome coronavirus 2; SF-36v2 = 36-Item Short Form Survey version 2; SJC = swollen joint count; TJC = tender joint count; V = Visit; VAS = visual analog scale.
Note:
All laboratory sample collections and assessments on dosing day may be performed predose, unless specified otherwise.
^{a}Final visit for participants who enrolled and were dosed in the OLE.
^{b}Final study visit for all participants who do not enroll or do not receive at least one dose in the OLE study.
^{c} To participate in study, a negative rapid SARS-CoV-2 viral test may be conducted prior to dosing.
^{d} If no symptoms are present, the exam can be limited to assessments required for ESSDAI and total joint count.
^{e} Serum chemistry, clinical chemistry, and hematology. Serum Uric Acid may be analyzed as part of the chemistry panel at Baseline, Week 24 and Week 48.
^{f} Anti-SSA (Ro), anti-SSB (La), and antinuclear antibody.
^{g} Plasma immunoglobulin levels (IgM, IgG, IgA), beta-2 microglobulin, and high-sensitivity C-reactive protein.
^{h} Exploratory biomarkers including but not limited to chemokine (C-X-C motif) ligand 13 (CXCL13) and soluble cluster of differentiation 40 ligand (sCD40L).

### Study Assessments and Procedures

Study procedures and their timing are summarized in the schedule of assessments (Table 7).

All screening evaluations must be completed and reviewed to confirm that potential participants meet all eligibility criteria.

In the event of a significant study-continuity issue (e.g., caused by a pandemic), alternate strategies for participant visits, assessments, medication distribution, and monitoring may be implemented.

Results of laboratory testing for pharmacokinetics (PK), pharmacodynamics (PD), or for research that could unblind the study will not be reported to investigative sites or other blinded personnel until the study has been unblinded.

### Screening

Table 3 summarizes the screening procedures for the study. More than one visit might be needed to complete screening. Patient-reported outcomes should be performed after obtaining informed consent but prior to other procedures for all study visits.

### Unscheduled Visits

Unscheduled visits are permitted. Repeat or unscheduled samples may be taken.

### Efficacy Assessments

Planned timepoints for all efficacy assessments are provided in the schedule of assessments (Table 7).

### ESSDAI

The ESSDAI is a systemic disease activity index that includes organ-by-organ definitions of disease activity (Seror et al, 2010). The ESSDAI grades disease activity in 12 domains (cutaneous, respiratory, renal, articular, muscular, peripheral nervous system, central nervous system, hematological, glandular, constitutional, lymphadenopathic, and biological). The weights of each domain were obtained by multiple regression modeling, using the Physician's Global Assessment of Activity as gold standard.

Each domain is weighted from 1 (Biologic domain) to 6 (Muscular domain) and has 3 or 4 levels of activity per domain, ranging from 0 (no activity) to 3 or 4 (severe activity).

The theoretical range of values for the ESSDAI is 0 to 123, with the final score being calculated as follows:

$$Final score = Sum of all 12 domain scores$$

$$Domain score = Activity level \times Domain weight$$

Low-activity status is defined as ESSDAI<5, moderate-activity as 5≤ESSDAI≤13, and severe activity as ESSDAI≥14 (Seror et al, 2016).

The ESSDAI is an appropriate primary endpoint because it is a validated and widely used measure of systemic disease activity in SS (Seror et al, 2015). It was found to have good construct validity with reliable scoring; in addition, systemic scores demonstrated good sensitivity to change in patients whose disease activity improves.

### ESSDAI [5] Response

The ESSDAI [5] refers to a 5-point reduction from baseline in ESSDAI score. Because improvements in continuous variables, such as the ESSDAI, can be difficult to interpret at the individual patient level, a minimal clinically important improvement (MCII) was developed to identify the minimal change of a continuous variable that enacts a meaningful clinical improvement within an individual (Kvien et al, 2017). While the MCII of an ESSDAI in SS has been identified to be at least 3 points, a retrospective examination of the EULAR Sjögren's cohort suggested the use of a 5-point MCII, underscoring the possible need for a higher MCII in patients with higher systemic activity (Seror et al, 2015; Seror et al, 2016).

### ESSPRI

The ESSPRI is a self-evaluation tool that was developed in a multicenter international cohort of 230 patients (Seror et al, 2011). The ESSPRI uses a 0 to 10 numerical analog scale (ranging from 0 [no symptoms] to 10 [maximal imaginable severity]), one for the assessment of each of the 3 domains: dryness, fatigue, and pain (articular and/or muscular). The weights of the domains are identical and the mean of the scores of the 3 domains represents the final score. The recall period is stated in each question as “the last 2 weeks”.

Sjögren's syndrome patients with exocrine dysfunction and severe subjective symptoms can be defined by an ESSPRI score of ≥5, which is considered as the cut-off point for “unsatisfactory symptom state” (Seror et al, 2016). The MCII in ESSPRI score is defined as a decrease of at least 1 point or 15% (Seror et al, 2016).

The ESSPRI is a validated and widely used tool (Seror et al, 2011; Seror et al, 2015). Although all 3 domains carry importance, the ESSPRI dryness domain has been shown to correlate with quality of sleep and anxiety and depression (Gandía et al, 2014) and overall quality of life (Schmalz et al, 2020).

Tender Joint Counts (TJC) and Swollen Joint Counts (SJC); 28-joint assessment

Arthralgia, morning stiffness, or synovitis have been reported to occur in over 50% of patients with primary SS (pSS), with about 16% experiencing frank non-erosive arthritis (Ramos-Casals et al, 2015). Additionally, 7% to 17% of RA patients have a diagnosis of secondary SS (sSS) (Young et al, 2000, Carmona et al, 2003). The 28-joint assessment will assess the following joints for tenderness and swelling: left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities, and left and right knee of the lower extremities. Each of the 28 joints will be evaluated for the presence of synovitis. At the start of the 28-joint count (prior to assessment of tenderness and swelling), participants will be asked if they have experienced or are experiencing pain in any of the 28 joints. Tender and swollen joint counts are widely used in clinical trials of rheumatologic disorders and are appropriate for use in this study because of the frequent involvement of joints in SS.

36-item Short Form Survey (SF-36)

The SF-36 (acute recall) is a 36-item general health status assessment that captures information about 8 health domains: Physical Functioning, Role Physical, Bodily Pain, General Health, Vitality, Social Functioning, Role Emotional, and Mental Health. The SF-36 provides scores for each domain as well as 2 psychometrically based summary scores: Physical Component Score (PCS) and Mental Component Score (MCS). The recall period for the acute version is one week (i.e., “last week”).

The SF-36 is a widely used, validated professional quality of life score that encompasses multiple domains and is sensitive to change (Hemingway et al, 1997). The PCS is derived from multiple physical domains and has been shown to be validated and responsive in related autoimmune diseases (Kosinski et al, 1999; Devilliers et al, 2015).

The SF-36 PCS score can comprehensively capture improvements in physical activity because its score based on the assessment of several components that are relevant to patients with SS (Sjögren's Foundation 2021).

### Functional Assessment of Chronic Illness Therapy (FACIT)-Fatigue

The FACIT-Fatigue is a participant-completed, 13-item questionnaire used to assess the impact of fatigue. The FACIT-Fatigue recall period is 7 days. Responses range from 0 (Not at all) to 4 (Very Much). To calculate the total score, the negatively stated items are reversed by subtracting the response from “4”. Final scores are the sum of the responses and range from 0 to 52. Higher scores indicate better quality of life. The questionnaire takes 5 to 10 minutes to complete.

### Visual Analog Scale (VAS) of Ocular, Oral, and Vaginal Dryness

The VAS Oral/Ocular/Vaginal consists of 3 instruments that use a continuous 100 mm VAS (0 mm=best, 100 mm=worst) to assess change in the severity of oral, ocular, and vaginal dryness throughout the study. The respondent is asked to place a line perpendicular to the VAS line at the point that represents the symptom intensity over the past 2 weeks. The VAS oral rates the question “How dry your mouth feels most of the time” (not dry at all 0 mm; dry as a desert 100 mm). The VAS ocular rates the question “How dry do your eyes feel most of the time” (not dry at all 0 mm; very dry 100 mm). The VAS vaginal ask respondents (as appropriate) to rate symptoms of vaginal dryness (no symptoms 0 mm; worst possible symptoms 100 mm). Each of the instruments takes less than 1 minute to complete.

### Patient Global Impression of Severity (PGIS)

The PGIS is a single-item questionnaire designed to capture the participant's perception of overall symptom severity over the past week on a 5-point categorical response scale (none, mild, moderate, severe, or very severe).

### Patient Global Impression of Change (PGIC)

The PGIC is a single-item questionnaire designed to capture the participant's perception of change in their overall symptom severity from starting the IP. Change in severity is captured using a 5-point scale (much better, a little better, no change, a little worse, or much worse).

### Stimulated Salivary Flow

Whole stimulated salivary flow will be measured to objectively assess functional changes in the salivary glands during treatment with Dazodalibep. Participants receiving standard of care for xerostomia at screening must discontinue use of pilocarpine or cevimeline for at least 12 hours and artificial saliva for at least 3 hours prior to saliva collection. Participants should be prohibited from eating or drinking for at least 90 minutes prior to saliva collection. To minimize diurnal variation, every attempt should be made to collect saliva at the same time of the day as the Day 1 assessment across all subsequent visits.

For whole stimulated salivary flow rate measurements, an approximately 5×5 cm parafilm square is rolled and given to the participants to be chewed like a gum at a rate of approximately 60 strokes per minute. The participant should be seated upright with eyes open and head tilted slightly forward and start chewing the parafilm for 60 seconds, at which point all the collected saliva should be spit in an extra (not pre-weighed) falcon tube, keeping the parafilm in the mouth. This first collection accustoms the participant to the procedure. Three rounds of salivary collection, each after 20 seconds of chewing, follow in a pre-weighed falcon tube. Those rounds are continuous, but timing should be stopped during the collection of the saliva and restart immediately after the saliva deposition in a pre-weighed falcon tube, for a total stimulation time of 60 seconds. If collection for 60 seconds is not feasible due to the participant's inability, the total collection time should be recorded. Total stimulated saliva collected is assessed by subtracting the final weight of the tube from the weight prior to collection.

### Physician Global Impression of Severity (MDGIS)

The MDGIS represents the Investigator's overall assessment of SS disease severity and is a 5-point categorical response scale (none, mild, moderate, severe, or very severe). If possible, the same Investigator should complete this assessment for the same participant throughout the study.

### Female Sexual Function Index (FSFI)

The FSFI was designed to define female sexual function in clinical and nonclinical samples by establishing clear endpoints and outcomes for female sexual function research (Rosen et al, 2000). The FSFI is 19-item, self-reported, and validated questionnaire assessing function over the past 4 weeks in the following domains: desire, arousal, lubrication, orgasm, satisfaction, and pain. Each question is scored on scale of 0 (no sexual activity) or 1 (maximal dysfunction) to 5 (no sexual dysfunction). The final score is computed in the following manner: to normalize each domain based on item number, the sum of each domain score is first multiplied by a domain factor ratio (0.6 for desire; 0.3 for arousal; 0.3 for lubrication; 0.4 for orgasm; 0.4 for satisfaction; and 0.4 for pain) and then the domain scores are added to produce a final score. This questionnaire may be completed by female participants.

### Clinical EULAR Sjögren's Syndrome Disease Activity Index (ClinESSDAI)

The ClinESSDAI is a validated SS disease activity index based on ESSDAI that excludes the biological domain and assigns different weights assigned to each domain. ClinESSDAI was developed to diminish possible associations between the B-cell biomarkers measured by the ESSDAI biological domain and clinical activity measures (Seror et al, 2016). The theoretical range of values for the ClinESSDAI is 0 to 135. Similar to ESSDAI, low-activity status is defined as <5, moderate-activity as 5≤ClinESSDAI≤13, and high-activity as ≥14 (Seror et al, 2016). ClinESSDAI has been validated and shown to correlate well with ESSDAI and is considered a useful tool to detect change independent of biological effect of the drug (Seror et al, 2016; Dumusc et al, 2018; Quartuccio et al, 2017).

### Ultrasound Imaging

### Salivary Gland

Ultrasound measurement of the parotid and submandibular glands may be performed in a subset of participants at visits indicated in Table 7. Participants with significant glandular disease will be selected from sites with expertise and equipment availability based on their willingness to participate. The glands above will be assessed in longitudinal and transverse planes with participants in supine position. After image capture and assessment of quality, images will be transmitted to a central vendor for scoring and data summation. Echostructure of each gland on B-mode images will be scored on a 5-point scale (0 to 4) as previously described (Gazeau et al, 2018). Grading criteria will be as follows:
Grade 0: normal homogenous gland
Grade 1: small hypoechoic areas with hyperechoic bands
Grade 2: multiple hypoechoic areas<2 mm
Grade 3: multiple hypoechoic areas 2 to 6 mm
Grade 4: multiple hypoechoic areas>6 mm

At each time point assessed, 4 scores will be collected, one for each parotid and one for each submandibular gland. The Salivary Gland Ultrasound score for each assessment will be the sum of these grades.

### Joint

Ultrasound evaluation of small peripheral joints may be performed at visits indicated in Table 7. Participants with possible articular involvement may be selected. A modified German 7-joint US scoring system will be used (Backhaus et al, 2009). After image capture and assessment of quality, images will be transmitted to a central vendor for scoring and data summation.

Grayscale (GS) ultrasound will be performed on the following clinically dominant hand and foot joints: wrist (dorsal, palmar, and ulnar planes), second and third MCP (MCP2 and MCP3, palmar plane) and PIP (PIP2 and PIP3, palmar planes), and second and fifth metatarsophalangeal (MTP2 and MTP5, dorsal plane) joints. In GS, each joint and each plane will be semi-quantitatively scored on the following scale of 0 to 3 for synovitis: Grade 0=absence of synovitis; Grade 1=mild synovitis (small hypoechoic/anechoic line beneath the joint capsule); Grade 2=moderate synovitis (joint capsule is elevated parallel to joint), and; Grade 3=severe synovitis (maximal distention of joint capsule). The total GS for synovitis score will be the sum of each joint or plane, for a score of 0-27.

Power doppler ultrasound will be performed on the following clinically dominant hand and foot joints: wrist (dorsal, palmar, and ulnar planes), second and third MCP (MCP2 and MCP3, palmar and dorsal planes) and PIP (PIP2 and PIP3, palmar and dorsal planes), and second and fifth metatarsophalangeal (MTP2 and MTP5, dorsal plane only) joints. Synovitis assessment using power doppler will be scored on a scale of 0 to 3 as follows: Grade 0=no IA color signal; Grade 1=mild synovitis (up to 3 color signals or 2 single and 1 confluent signal in IA space); Grade 2=moderate synovitis (greater than Grade 1 but color occupying less than 50% of IA space), and; Grade 3=severe synovitis (greater than 50% color in IA space). The total GS for synovitis score will be the sum of each joint or plane, for a score of 0-39.

The total synovitis score will be the sum of GS and power doppler scores, for range of 0-66.

### Physical Examinations

Full physical examination and symptom-driven physical exams will be performed per the screening schedule (Table 3) and schedule of assessments (Table 7). For symptom-driven physical exams, the exam can be limited to examinations needed to assess ESSDAI/TJC and SJC if no symptoms are present. A full physical examination does not require internal pelvic examination for women or rectal examination for men or women.

### Vital Signs

Vital signs, including systolic and diastolic BP (mmHg), pulse rate (beats/min), respiratory rate (breaths/min), body temperature (C), and body weight (kg) will be measured using clinically acceptable methods and devices as defined in the screening schedule (Table 3) and schedule of assessments (Table 7). Vital signs will be measured in a seated position after 5 minutes rest and will include temperature, systolic and diastolic BP, and pulse and respiratory rate.

### Electrocardiograms

A single 12-lead ECG with the participant in the supine position will be performed as specified in the screening schedule (Table 3) and schedule of assessments (Table 7).

Electrocardiogram measurement should be delayed by at least 10 minutes if performed after phlebotomy.

Each ECG will include ventricular heart rate and intervals (PR, QRS, QT, RR).

### Clinical Safety Laboratory Tests

Blood and urine samples will be collected for laboratory safety tests as specified in the screening schedule (Table 3) and schedule of assessments (Table 7).

Abnormal laboratory findings associated with the underlying disease may not be considered clinically significant.

All laboratory tests with values considered clinically significantly abnormal during participation in the study or within the protocol follow-up period after the last dose of IP should be repeated until the values return to normal or baseline or are no longer considered clinically significant. All protocol-required laboratory tests must be conducted in accordance with the laboratory manual and the screening schedule (Table 3) and schedule of assessments (Table 7).

Clinical safety laboratory tests are specified in Table 8 Additional tests may be performed at any time during the study.

TABLE 8
Exemplary clinical safety laboratory tests

| Laboratory | |
|---|---|
| Tests | Parameters |
| Hematology | Platelet count |
| | RBC count |
| | RBC indices |
| | MCV |
| | MCH |
| | WBC count with differential: |
| | Neutrophils |
| | Lymphocytes |
| | Monocytes |
| | Eosinophils |
| | Basophils |
| | Hemoglobin |
| | Hematocrit |
| | Immunoglobulins (IgG, IgM, IgA) |
| Clinical | Blood urea nitrogen (BUN) |
| chemistry ª | Potassium |
| | Creatinine |
| | Sodium |
| | Calcium |
| | Chloride |
| | Bicarbonate |
| | Phosphorus |
| | Glucose |
| | AST/SGOT |
| | ALT/SGPT |
| | Alkaline phosphatase |
| | Total and, if total value is abnormal, fractionation into direct and indirect bilirubin |
| | Gamma-glutamyltransferase |
| | Albumin |
| | Total protein |
| | Creatinine kinase |
| | Calculated eGFR |
| | Uric acid |
| | Total cholesterol |
| | Low-density lipoprotein (LDL) |
| | High-density lipoprotein (HDL) |
| | Triglycerides |
| Urine testing | Specific gravity, pH, glucose, protein, blood, ketones by dipstick |
| | Microscopic examination (crystals, casts, white blood cells, red blood cells) if blood |
| | or protein is abnormal) |
| | Protein: creatinine ratio |
| Pregnancy | Highly sensitive serum β-hCG pregnancy test (as needed for women of childbearing |
| testing | potential) at screening; local urine pregnancy testing for all subsequent testing ^{b} |
| Other screening | Follicle-stimulating hormone (as needed to confirm menopause only) |
| tests | Serology: Hepatitis B, Hepatitis C; HIV-1; HIV-2 |
| | SARS-COV-2 rapid virologic test ^{c} |
| | TB test (IGRA) |
| | Coagulation panel (prothrombin time, INR, PTT) |

ALT = alanine aminotransferase;
AST = aspartate aminotransferase;
BUN = blood urea nitrogen;
eGFR = estimated glomerular filtration rate;
hCG = human chorionic gonadotropin;
HIV = human immunodeficiency virus;
Ig = immunoglobulin;
IGRA = interferon gamma release assay;
INR = international normalized ratio;
IRB/IEC = institutional review board/independent ethics committee;
MCH = Mean corpuscular hemoglobin;
MCV = mean corpuscular volume;
PTT = partial thromboplastin time;
RBC = red blood cell;
SARS-COV-2;
severe acute respiratory syndrome coronavirus 2;
SGOT = serum glutamic-oxaloacetic transaminase;
SGPT = serum glutamic-pyruvic transaminase;
TB = tuberculosis;
WBC = white blood cell.
^{a} Changes in liver chemistries that may indicate liver injury (possible Hy'slaw) must be reported to the Sponsor. See Section 10.4 (Appendix 4).
^{b} Local urine pregnancy testing will be standard for the protocol unless serum testing is required by local regulation or IRB/IEC.
^{c} Individuals should have a negative virologic test for SARS-COV-2 on the day of randomization. The specific test used is in accordance with availability at the site.

### Pregnancy Testing

Serum β-hCG pregnancy test(s) will be completed for all females of childbearing potential during the screening period (before Day 0) and by urine pregnancy test at the visits in the screening schedule (Table 3) and schedule of assessments (Table 7).

### Additional Disease-Related Assessments

Central laboratory assessments related to SS described below will be conducted according to the screening schedule (Table 3) and schedule of assessments (Table 7).

### Autoantibody Testing

Serum will be collected to assess the presence of anti-SSA (Ro), anti-SSB (La), antinuclear antibodies, and RF.

### Inflammatory Markers

Whole blood, plasma, serum, and urine will be collected to assess levels of plasma immunoglobulins (IgM, IgG, and IgA), β-2 microglobulin, high-sensitivity CRP, serum C3, C4, free light chains, cryoglobulins, and serum and urine immunofixation.

### Pregnancy

Details of all pregnancies in female participants and, if indicated, female partners of male participants will be collected after the start of IP through the end of the study.

Any female participant who becomes pregnant while participating in the study will discontinue IP but will continue to be followed through the end of the study.

The participant/pregnant female partner will be followed to determine the outcome of the pregnancy.

### Pharmacokinetics

Plasma samples to determine the concentration of Dazodalibep will be obtained according to the visits specified in Table 7 and assessed using a validated assay.

The timing of sampling may be altered during the course of the study based on newly available data (e.g., to obtain data closer to the time of peak plasma concentrations) to ensure appropriate monitoring.

Samples will be used to evaluate the PK of Dazodalibep. Samples collected for analyses of Dazodalibep plasma concentration may also be used to evaluate safety or efficacy aspects during or after the study.

Investigational product concentration information that may unblind the study will not be reported to investigative sites or blinded personnel until the study has been unblinded to the specific group to whom the data will be provided.

### Pharmacodynamics and Target Engagement

Whole blood, plasma, and serum will be collected to evaluate PD of Dazodalibep on CD40/CD40L pathways in participants with SS upon treatment with Dazodalibep. Samples will be collected according to Table 7.

PD biomarkers of disease activity to be assessed in this study may include but are not limited to:
IgG and IgM RF autoantibodies (PD)
sCD40L (TE)
CXCL13 (PD)
Flow Cytometry of B-cell subsets

Samples will be analyzed using a qualified analytical method.

### Mechanistic/Disease Biomarkers

These data will be analyzed using descriptive statistics.

Whole blood, plasma, and serum will be collected to assess levels of plasma immunoglobulins (IgM, IgG, and IgA), beta-2 microglobulin, high-sensitivity CRP, serum C3, C4, free light chains, cryoglobulins, and serum for anti-SSA, anti-SSB, and IgG.

### Exploratory Flow Cytometry

Whole blood samples may be collected from participants for the assessment of changes in the number, activation status, and frequency of major leukocyte populations, including B lymphocytes using flow cytometry.

These data may be analyzed using descriptive statistics.

### Exploratory Biomarker Samples

Serum, plasma, whole blood, and saliva will be collected to measure changes in exploratory biomarkers of disease activity or drug response.

These data may be analyzed using descriptive statistics.

### RNA PAXgene

Blood RNA will be used to measure the expression levels of genes associated with disease activity, specific cell types (e.g., plasma cell gene signature, T follicular helper gene signature), and signaling (e.g., the CD40L/CD40 pathway).

### Saliva Sampling

Samples collected for assessment of whole stimulated salivary flow will be used for biomarker measurements. Saliva samples will be collected according to Table 7.

### Immunogenicity Assessments

Plasma samples for immunogenicity (ADA to Dazodalibep) may be taken prior to IP administration according to the visits specified in Table 3 and Table 7, and assessed using a validated immunoassay.

Antibodies to Dazodalibep will be evaluated in plasma samples collected from all participants according to Table 7. Additionally, plasma samples should also be collected at the final visit from participants who discontinued IP or were withdrawn from the study.

Plasma samples may be screened for antibodies binding to Dazodalibep and the titer of confirmed positive samples may be reported. Other analyses may be performed to verify the stability of antibodies to Dazodalibep and/or further characterize the immunogenicity of Dazodalibep.

The detection and characterization of antibodies to Dazodalibep will be performed using a validated assay method. Antibodies may be further characterized and/or evaluated for their ability to neutralize the activity of the study intervention(s). Samples may be stored for a maximum of 15 years (or according to local regulations) following the last participant's last visit for the study to enable further analysis of immune responses to dazodalibep.

### Analysis Sets

Full analysis set: The full analysis set (FAS) will include all randomized participants who receive any dose of IP in the study. Participants will be analyzed according to the treatment randomized. The efficacy analysis will be based on the FAS.

PK analysis set: The PK analysis set will include all participants who receive any dose of Dazodalibep in the study and have at least one quantifiable serum PK observation post first dose. Participants will be analyzed according to the treatment that they actually received. The PK analysis will be based on the PK analysis set.

### Immunogenicity Analysis

Number and percentage of participants who develop ADA and ADA titer will be summarized by visit and by treatment group.

### Pharmacokinetics Analysis

Descriptive statistics of the serum Dazodalibep concentration will be tabulated by visit and by treatment group.

### Pharmacodynamics Analysis

The PD biomarkers results, as well as their changes from baseline, will be summarized descriptively by visit and treatment group.

### Exploratory Endpoints Analyses

The exploratory analyses are summarized.

### Example 2—A Phase 3, Randomized, Double-Blind, Placebo-Controlled Study to Evaluate the Efficacy and Safety of Dazodalibep in Participants with Sjögren's Syndrome with Moderate-to-severe Symptom State

A phase 3, randomized double-blind, placebo-controlled study to evaluate the efficacy and safety of Dazodalibep in participants with Sjögren's Syndrome (SS) with moderate-to-severe symptom state is disclosed herein.

### Objectives:

Study objectives are presented in Table 9.

TABLE 9
Exemplary study objectives

| | |
|---|---|
| Objectives | Endpoints |

Primary

| | |
|---|---|
| To evaluate the effect of dazodalibep on systemic | Change from baseline in ESSPRI |
| manifestations of SS in participants with moderate-to-severe | score at week 48 (Plan B) |
| symptom state | Change from baseline in Diary for |
| | Assessing Sjogren'sPatient Reported |
| | Index (DASPRI) score at Week 48 |
| | (Plan A) |

Key Secondary

| | |
|---|---|
| To evaluate the effect of dazodalibep on patient reported | Proportion of participants achieving a |
| | meaningful improvement in DASPRI |
| | (Plan A) or ESSPRI (Plan B) |
| | response, defined as a decrease of at |
| | least 1.5 points from baseline in the |
| | ESSPRI score, without premature |
| | discontinuation from treatment and |
| | without receiving rescue or |
| | potentially confounding therapy. |
| outcomes (PROs) in participants with SS | Change from baseline in DASPRI |
| | Dryness (Plan A) or ESSPRI fatigue |
| | domain score Dryness (Plan B) at |
| | Week 48 |
| | Change from baseline in PROMIS- |
| | Fatigue SF-10a at Week 48 (Plan A |
| | and Plan B) |
| | Change from baseline in DASPRI |
| | Pain (Plan A) or ESSPRI Pain (Plan |
| | B) at Week 48 |
| | Change from baseline in 36-item |
| | Short Form Survey (SF 36-36) |
| | Physical Component Summary (PCS) |
| | score at Week 48 (Plan B only) |

Secondary

| | |
|---|---|
| To evaluate the effect of dazodalibep on measures of | Change from baseline in DASPRI |
| systemic activity, PROS, and salivary flow in participants | total score (Plan A) or ESSPRI total |
| with SS | score (Plan B) at Week 12 and Week |
| | 24 |
| | Change from baseline in DASPRI |
| | Fatigue (Plan A) or ESSPRI Fatigue |
| | (Plan B) at Week 48 |
| | Change from baseline in total |
| | stimulated salivary flow at Week 48 |
| | (Plan A and B). |

Exploratory

| | |
|---|---|
| To assess the immunogenicity of multiple doses of | Proportion of participants who |
| dazodalibep in participants with SS | develop anti-drug antibodies (ADAs) |
| To evaluate biomarkers of inflammation and | over time |
| autoantibodies in participants with SS upon | Change from baseline in levels of |
| treatment with dazodalibep | markers of inflammation |
| To evaluate dazodalibep target engagement (TE) | (immunoglobulins, β-2 |
| To characterize the PK of dazodalibep in | microglobulin, and C-reactive protein |
| participants with SS | [CRP].) |
| To evaluate the PD of dazodalibep in participants | Pharmacokinetic concentration of |
| with SS | dazodalibep in plasma. |
| To evaluate the effects of dazodalibep on systemic | Change from baseline in rheumatoid |
| disease activity as well as in joints and salivary and | factor (RF) autoantibodies. |
| lacrimal function in participants with SS. | Change from baseline in levels of |
| To evaluate changes in key subjective complaints | biomarkers including but not limited |
| associated with SS. | to peripheral blood flow cytometry for |
| To assess effects of dazodalibep on blood gene | changes in B-cell and T-cell subsets, |
| expression | serum CXCL 13 |
| To characterize exploratory biomarkers of disease | Change from baseline in participant- |
| and response to treatment | reported oral, ocular, skin, or genital |
| | dryness (NRS from DASPRI) at Week |
| | 48 (Plan A) |
| | Proportion of participants with > 0.5 |
| | mL/60 seconds total stimulated |
| | salivary flow |
| | Change from baseline in Schirmer's |
| | test |
| | Change from baseline in total |
| | unstimulated salivary flow |
| | Proportion of participants achieving |
| | STAR (where appropriate) |
| | Change from baseline in Physician |
| | Global Impression of Severity |
| | (MDGIS) |
| | Change from baseline PGIS overall |
| | SS symptoms, dryness, pain, and |
| | fatigue score |
| | Change from baseline in PGIC overall |
| | SS symptoms, dryness, pain, and |
| | fatigue score |
| | Change from baseline in SF-36 MCS |
| | Change from baseline in EQ-5D-5L |
| | Change from baseline in Female |
| | Sexual Function Index (FSFI) at |
| | Week 48 (where appropriate) |
| | Change from baseline in ESSDAI |
| | score |
| | Change in baseline in biomarker |
| | levels |
| | Change in baseline in blood |
| | transcriptome |

Plan A = U.S., Plan B = ex-U.S.

### Study Design

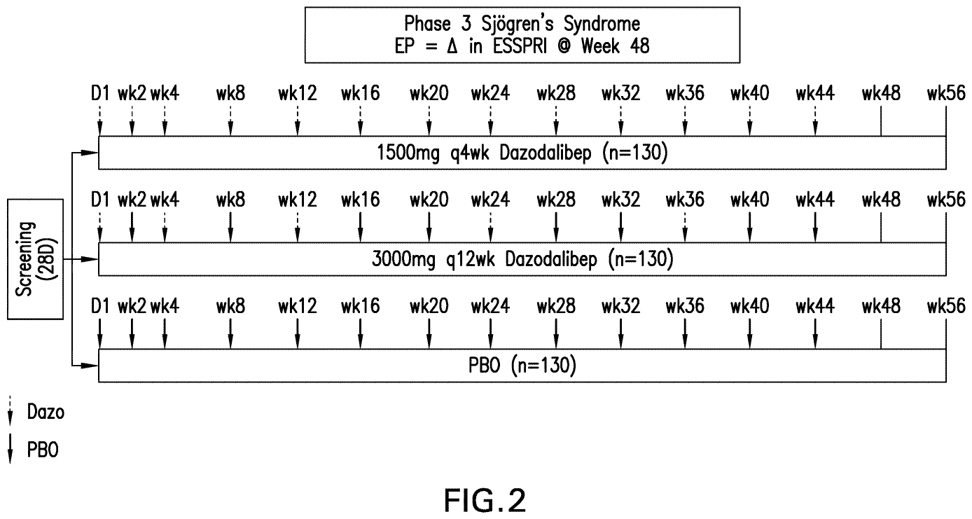

This study is a Phase 3, randomized, double-blind, placebo-controlled study to evaluate the efficacy and safety of Dazodalibep in participants aged≥18 diagnosed with Sjögren's syndrome (SS) with moderate-to-severe symptom state. The study will enroll SS participants with moderate-to-severe symptomatic activity (Seror et al, 2016) as defined by ESSPRI score≥5, and with a low systemic disease activity defined by ESSDAI score<5. The study design is provided in FIG. 2.

Individuals will undergo a screening period of up to 28 days followed by randomization and treatment through the Week 44 visit. Approximately 435 participants will be randomized (1:1:1) to dazodalibep Dose 1, dazodalibep Dose 2, or placebo as follows:
Dazodalibep Dose 1:1500 mg intravenously (IV) at Weeks 0, 2, and 4 then once every 4 weeks (Q4W) (13 total doses: 13 doses of dazodalibep) (n=145)
Dazodalibep Dose 2:3000 mg IV at Weeks 0, 4, and 12 then once every 12 weeks (Q12W) (13 total doses: 5 doses of dazodalibep; 8 doses of placebo) (n=145)
Placebo (13 total doses: 13 doses of placebo) (n=145)

Randomization will be stratified by screening ESSPRI score of <7.5 or ≥7.5, by coexisting RA or systemic lupus erythematosus (SLE) (yes versus no), and by geographical region (North America and Europe versus Japan versus rest of world). Participants will receive randomized treatment (dazodalibep or placebo) through Week 44. At the Week 44 visit, eligible participants may provide written informed consent to screen for an open-label extension (OLE) study for receipt of open-label dosing with dazodalibep. Participants ineligible for or not wishing to enroll in the OLE extension will have a final study visit at Week 56 to complete 12 weeks of follow-up after the last dose of investigational product (IP). The expected full duration of each individual's participation in this study, including screening, but not the OKE, is up to 420 days.

Eligible participants interested in participating in the OLE will complete participation in this Phase 3 study at the Week 48 visit, after completion of all assessments. These Week 48 visit values will serve as baseline (Day 1) for the OLE study, and Dose 1 of open-label dazodalibep will be administered at or at approximately the Week 48 visit, their final visit in this Phase 3 study. All participants will be followed to per-protocol completion of study (Week 48 visit for those who enroll in and are dosed in the OLE study and the Week 56 visit for those not dosed in the OLE study). Participants who discontinue treatment will be followed until the Week 48 visit or 12 weeks after the last dose of IP, whichever is longer, unless the participants withdraw from the study or are lost to follow-up. Participants who refuse to participate in some aspects of the study should, unless consent for all participation is withdrawn, participate in those aspects for which they continue to consent. All participants, including those who enroll in the OLE, will remain blinded to their treatment assignment in Phase 3 until the Phase 3 study is complete.

The primary endpoint for the US is DASPRI (Plan A), and the primary endpoint for ex-US countries is ESSPRI (Plan B). The primary objective of this study is to evaluate the effect of dazodalibep on patient-reported symptoms of SS in participants with moderate-to-severe symptom state. This will be assessed by evaluating the change from baseline in ESSPRI (Plan A) or DASPRI (Plan B) score at Week 48, measures of patients' symptoms in primary SS. Both the ESSPRI and DASPRI assessments will be completed by participants in all regions.

### Study Population

### Inclusion Criteria

To be included in this study, individuals may satisfy all the following criteria:
1. Adults, ≥18 years at time of informed consent.
2. Diagnosed with SS by meeting the 2016 American College of Rheumatology (ACR)/EULAR Classification Criteria. If SS diagnosis is based on positive anti-Ro autoantibody, anti-Ro positivity will be confirmed by central lab.
3. Have an ESSPRI score of ≥5 at screening despite symptomatic or local therapy.
4. Have an ESSDAI score of <5 at screening.
5. Positive for either anti-Ro autoantibodies or rheumatoid factor (RF), or both at screening.
6. Residual salivary gland function as defined by whole stimulated salivary flow>0.1 mL/min.
7. Females of childbearing potential who are sexually active with a non-sterilized male partner must use a highly effective method of contraception from signing the informed consent form (ICF) and must agree to continue using such precautions through the end of the study or 3 months after last IP administration (if participant withdraws from the study); cessation of contraception after this point should be discussed with a responsible physician. A woman of childbearing potential must have a negative highly sensitive serum pregnancy test (as required by local regulations) in screening and must have a negative urine pregnancy test on the day of dosing prior to each dose of IP. Highly effective methods of contraception include:
   a. Combined (estrogen and progestogen containing) hormonal contraception associated with inhibition of ovulation:
      i. Oral
      ii. Intravaginal
      iii. Transdermal
      iv. Injectable
   b. Progestogen-only hormonal contraception associated with inhibition of ovulation:
      i. Oral
      ii. Injectable
      iii. Implantable
   c. Intrauterine device
   d. Intrauterine hormone-releasing system
   e. Bilateral tubal occlusion
   f. Azoospermic partner (vasectomized or due to a medical cause)
      i. Azoospermia is a highly effective contraceptive method provided that the partner is the sole sexual partner of the woman of childbearing potential and the absence of sperm has been confirmed. If not, an additional highly effective method of contraception should be used. Spermatogenesis cycle is approximately 90 days.
   g. Sexual abstinence
      i. Sexual abstinence is considered a highly effective method only if it is the preferred and usual lifestyle of the participant and the participant agrees to refrain from heterosexual intercourse from screening through the end of the study follow-up. Periodic abstinence, the rhythm method, and the withdrawal method are not acceptable methods of contraception. A recommendation that the female partners (of childbearing potential) of male study participants should use a highly effective method of contraception other than a barrier method should be made.
         1. Females of childbearing potential are defined as those who are not surgically sterile (surgical sterilization includes bilateral tubal ligation, bilateral oophorectomy, or hysterectomy) or those who are not postmenopausal (defined as 12 months with no menses without an alternative medical cause).
         2. Vasectomized partner is a highly effective birth control method provided that the partner is the sole sexual partner of the woman of childbearing potential trial participant and that the vasectomized partner has received medical assessment of the surgical success.
8. Non-sterilized male participants who are sexually active with a female partner of childbearing potential must use a male condom with spermicide and refrain from donating fresh unwashed semen from Day 1 through the end of the study. His female partner should also be advised of the benefit to use a highly effective method of contraception, as a condom may break or leak.
9. Vaccinated against severe acute respiratory syndrome coronavirus 2 (SARS-COV-2) according to current local authority guidelines, if any, at least 2 weeks prior to screening unless the participant refuses vaccination. Initial or subsequent COVID-19 vaccine administration is permitted during the study as long as it is not administered during the screening period or within a week after Dose 1; if vaccine is to be administered during this window, screening should be delayed to complete vaccination.
10. Meets all of the following tuberculosis (TB) criteria:
   a. No history of latent or active TB prior to screening, except for latent TB with documented completion of locally appropriate treatment.
   b. No signs or symptoms suggestive of active TB from medical history or physical examination.
   c. No recent (≤12 weeks of screening) close contact with a person with active TB (close contact is defined as ≥4 hours/week or living in the same household OR in a house where a person with active TB is a frequent visitor).
   d. Negative Interferon Gamma Release Assay (IGRA) test result for TB at screen unless previously treated as per Inclusion Criterion 10 (a). Subjects with an indeterminate test result can repeat the test, but if the repeat test is also indeterminate, they are excluded.
   e. A chest radiograph (obtained during the screening period or any time within 12 weeks prior to screening) with no evidence of current active TB or other infection, or prior TB, malignancy, or clinically significant abnormalities suggesting an active process (unless due to SS).

### Exclusion Criteria

If an individual meets any of the following criteria, he or she is ineligible for this study:
1. Individuals with medical history of confirmed deep venous thrombosis, pulmonary embolism, or arterial thromboembolism within 2 years of screening.
2. History or presence of concomitant polymyositis or dermatomyositis or systemic sclerosis.
3. Active malignancy or history of malignancy within the last 5 years, except as follows:
   a. In situ carcinoma of the cervix treated with apparent success with curative therapy≥12 months prior to screening; or,
   b. Cutaneous basal cell carcinoma following presumed curative therapy.
4. Individuals who are pregnant or lactating or planning to become pregnant during the study.
5. Individuals with known history of severe allergy or reaction to any component of the IP formulation or to any other biologic therapy.
6. Individuals with any severe or life-threatening cardiovascular, (including vasculitis), respiratory, endocrine, gastrointestinal, hematological, neurological, psychiatric, or systemic disorder or any other condition that, in the opinion of the Investigator, would place the individual at unacceptable risk of complications, interfere with evaluation of the IP.
7. Individuals who are unable or unwilling to comply with protocol requirements (e.g., active drug or alcohol abuse or for other reasons), including the completion of the DASPRI.
8. Individuals who have a positive test for, or have been treated for, hepatitis B, hepatitis C, or human immunodeficiency virus (HIV) infection. A positive test for hepatitis B infection at screening is defined as: (1) positive for hepatitis B surface antigen (HBsAg); or (2) positive for hepatitis B core antibody (HBcAb). Patients HBsAg negative, hepatitis B surface antibody (HBsAb) positive and HBcAb negative due to vaccination are eligible for the study. Individuals with a positive test for or a history of treatment for hepatitis C are excluded unless they have a documented sustained viral response to antiviral drugs approved for the treatment of hepatitis C, defined as an undetectable viral level of hepatitis C RNA at least 24 weeks following completion of therapy. Individuals with advanced fibrosis or cirrhosis due to hepatitis C should not be enrolled.
9. Individuals with a positive test for SARS-COV-2 on the day of randomization or symptoms suggestive of SARS-COV-2 at randomization or significant exposure to coronavirus disease 2019 (COVID-19) within 10 days prior to randomization. Individuals with COVID-19 or COVID-19 exposure can delay randomization for 10 days and randomize once recovered; otherwise, they will need to rescreen.
10. Individuals with:
   a. A history of more than one episode of herpes zoster and/or any opportunistic infection in the last 12 months (see Table 10), with the exception of non-invasive herpes simplex at any site, oral candidiasis, vaginal candidiasis, or cutaneous fungal infections, which are permitted within the prior 12 months unless of unusual severity.
   b. Active infections requiring systemic treatment at the time of screening or through randomization, or history of more than 2 infections requiring IV antibiotics within 12 months prior to screening.
11. Individuals who have received a live (attenuated) vaccine within the 4 weeks prior to randomization or plan to receive a live vaccine during their participation in the study. Non-live vaccines are permitted during the study (see Inclusion Criterion 9 for COVID-19 vaccines); however, for subjects who plan to receive a vaccine within a month after dose 1, completing vaccination prior to starting dosing should be considered.
12. Last administration of experimental or investigational biologic or oral agents (other than those listed in Exclusion Criterion 16)<6 months prior to screening.
13. Individuals who have had previous treatment with any biologic B-cell-depleting therapy (e.g., rituximab, ocrelizumab, inebilizumab, ofatumumab, or ianalumab) within 12 months or other B-cell-targeting therapy (e.g., belimumab)<3 months prior to screening.
14. Inhaled, intranasal, or topical corticosteroids are allowed provided doses are expected to be stable during the study.
15. Individuals treated with systemic corticosteroids for indications other than SS, RA, and SLE for more than a total of 2 weeks within 6 months prior to screening.
16. Use of the following medications:
   a. Antimalarials (e.g., chloroquine, hydroxychloroquine, quinacrine) if they have been initiated or if the dose has changed within 8 weeks prior to screening or during the screening period.
   b. Oral, intramuscular, IV, or intra-articular corticosteroids within 4 weeks prior to screening. Inhaled, intranasal, or topical corticosteroids are allowed provided doses are expected to be stable during the study.
   c. Methotrexate, azathioprine, leflunomide, mycophenolate mofetil (MMF), other DMARD, immunosuppressant, biologics, or antiproliferative agents if last dose was taken within:
      i. 4 weeks prior to screening; or,
      ii. Drug-specific 5 half-lives elimination period (if longer than 4 weeks)
   d. Any medication that, in the opinion of the Investigator, would interfere with evaluation of the IP or interpretation of study results.
   e. Any increase or initiation of new doses of cyclosporine eye drops (Restasis®) or lifitegrast (Xiidra®) or other any topical (ophthalmic) anti-inflammatory/immunomodulatory eyedrops within 2 weeks prior to screening through randomization (Day 1).
   f. The use of medications for the treatment of underlying autoimmune disease not listed above, including herbal or homeopathic remedies such as sinomenine, tripterium glycosides, or total glucosides of peony, should be discussed with the Medical Monitor during screening to determine participant eligibility.
   g. Use of herbal or homeopathic remedies for underlying rheumatological conditions, such as sinomenine, tripterygium glycosides, or total glucosides of peony, within 4 weeks prior to screening
17. Individuals who have received previous treatment with anti-CD40L compounds at any time before screening.
18. Individuals with blood tests, at screening, of any of the following:
   a. Aspartate aminotransferase (AST)>2× upper limit of normal (ULN)
   b. Alanine aminotransferase (ALT)>2×ULN
   c. Total bilirubin (TBL)>2×ULN, unless Gilbert's syndrome is documented in the medical history
   d. Hemoglobin<90 g/L
   e. Neutrophils<1.0×10⁹/L Lymphocytes<0.5×10⁹/L f.
   g. Platelets<100×10⁹/L
   h. International normalized ratio (INR) for prothrombin time>1.3×ULN

TABLE 10
Exemplary infections that meet exclusion criteria

| Definite ^{a,b} Opportunistic Infection | Probable ^{c} Opportunistic Infection |
|---|---|
| Pneumocystis jirovecii | Paracoccidioides infections |
| BK virus disease, including polyomavirus-associated nephropathy | Penicillium marneffei (talaromyces) |
| CMV disease | Sporothrix schenckii |
| Post-transplant lymphoproliferative disorder (EBV) | Cryptosporidium species |
| Progressive multifocal leukoencephalopathy | (chronic disease only) |
| Bartonellosis (disseminated disease only) | Microsporidiosis |
| Blastomycosis | Leishmaniasis (visceral only) |
| Toxoplasmosis | Trypanosoma cruzi infection |
| Coccidioidomycosis | (Chagas's disease) (disseminated disease only) |
| Histoplasmosis | Campylobacteriosis (invasive disease only) |
| Aspergillosis (invasive disease only) | Shigellosis (invasive disease only) |
| Candidiasis (invasive disease or pharyngeal) | Vibriosis |
| Cryptococcosis | (invasive disease due to Vibrio vulnificus) |
| Other invasive fungi: Mucormycosis (zygomycosis) | HCV progression |
| (Rhizopus, Mucor, and Lichtheimia), | |
| Scedosporium/Pseudallescheria boydii, Fusarium) | |
| Legionellosis | |
| Listeria monocytogenes (invasive disease only) | |
| TB | |
| Nocardiosis | |
| Non-tuberculous mycobacterium disease | |
| Salmonellosis (invasive disease only) | |
| HBV reactivation | |
| Herpes simplex (invasive disease only) | |
| Herpes zoster (any form) | |
| Strongyloides | |
| (hyperinfection syndrome and disseminated forms only) | |

CMV = cytomegalovirus;
EBV = Epstein-Barr virus;
HBV = hepatitis B virus;
HCV = hepatitis C virus;
TB = tuberculosis.
Source: Winthrop et al 2015.
^{a} Generally does not occur in the absence of immunosuppression, but whose presence indicates a potential or likely alteration in host immunity.
^{b} Can occur in patients without recognized forms of immunosuppression, but whose presence indicates a potential or likely alteration in host immunity.
^{c} Published data are currently lacking, but expert opinion believes that risk is likely elevated in the setting of biologic therapy.

### Lifestyle Consideration

Meat and dietary restrictions: There are no dietary or fasting restrictions during the study.

Activity: There are no activity restrictions for this study.

### Criteria for Temporarily Delaying Randomization

Individuals with COVID-19 or COVID-19 exposure can delay randomization for 10 days and randomize once recovered; otherwise, they will need to rescreen (See Exclusion Criterion 9).

### Study Interventions and Concomitant Therapy

### Investigational Product Administered

Preparation of IP will be performed by an unblinded pharmacist/IP manager or study-site staff member. Administration of IP will be performed by a blinded administrator. The prepared IP must be covered with a bag prior to providing it to the blinded administrator. Each subject must receive the entire volume of IP solution in the IV bag over at least 90±10 minutes (approximately 2.8 mL/min); a 90 minute infusion time should be used unless there is reason for delay, such as infusion-related reaction or participant's personal need for a break in infusion (infusion start and stop times must be recorded in EDC, including timing of and reasons for break in infusion).

Table 11 includes a description of the Ips used in this study, their dose formulation, unit dose strength, dosage level, route of administration, use, sourcing, and packaging.

TABLE 11
Exemplary descriptions of the IPs used in this study

| Intervention | | | |
|---|---|---|---|
| Name | Dazodalibep 1500 mg | Dazodalibep 3000 mg | Placebo |
| Dose | 100 mg/mL of dazodalibep | 100 mg/mL of dazodalibep in 10 | 10 mM sodium |
| Formulation | in 10 mM sodium | mM sodium phosphate | phosphate |
| | phosphate | monobasic/dibasic, 250 mM | monobasic/dibasic, 250 |
| | monobasic/dibasic, 250 | sucrose, and 0.02% | mM sucrose, and 0.02% |
| | mM sucrose, and 0.02% | (weight/volume) poloxamer 188, | (weight/volume) |
| | (weight/volume) poloxamer | pH 7.4. | poloxamer 188, pH 7.4. |
| | 188, pH 7.4. | | |
| Type | Drug | Drug | Placebo |
| Unit Dose | 100 mg/mL dazodalibep | 100 mg/mL dazodalibep nominal | Not applicable |
| Strength(s) | nominal | | |
| Dosage Level(s) | 1500 mg | 3000 mg | Not applicable |
| Route of | IV infusion | IV infusion | IV infusion |
| Administration | | | |

### Blinding and Masking

### Blinding

Participants will be randomly assigned in a 1:1:1 ratio to study arms (see Table 12).

TABLE 12
Exemplary study arm(s)

| Arm Title | 1500 mg Q4W dazodalibep | 3000 mg Q12W dazodalibep | Placebo |
|---|---|---|---|
| Arm | Participants will receive | Participants will receive | Participants will receive |
| Description | dazodalibep 1500 mg IV at | dazodalibep 3000 mg IV at | placebo IV at Weeks 0, 2, |
| | Weeks 0, 2, and 4 then Q4W | Weeks 0, 4, and 12 then Q12W | and 4, then Q4W via a 90 |
| | via a 90 minute infusion for a | via a 90-minute infusion for a | minute infusion for a total |
| | total of 13 total doses | total of 13 total doses (5 doses of | of 13 total doses |
| | | dazodalibep and 8 doses of | |
| | | placebo) | |
| Associated | Dazodalibep 1500 mg | Dazodalibep 3000 mg, placebo | Placebo |
| Intervention | | | |
| Labels | | | |

IV = intravenous;
Q4W = once every 4 weeks;
Q12W = once every 12 weeks.

### Dose Modification

Dose adjustment of IP is not allowed in this study.

Continued Access to IP after the End of the Study

Participants will receive randomized treatment (Dazodalibep or placebo) through Week 44. At the Week 44 visit, eligible participants may screen for an OLE study for receipt of open-label dosing with Dazodalibep starting after the completion of this study at Week 48.

The schedule of study assessments during the treatment period is provided in Table 13. Prior and Concomitant Therapy

Any medication or vaccine (including over-the-counter or prescription medicines, recreational drugs, vitamins, and/or herbal supplements) or other specific categories of interest that the participant has received in the previous 3 months or 12 months for cDMARD, biologic/other immunosuppressive agents, or any other Sjögren's-related medications (for example, local or symptomatic treatments), is receiving at the time of enrollment, or receives during the study must be recorded along with:
5. Name and indication
6. Reason for use
7. Dates of administration including start and end dates
8. Dosage information including dose and frequency

### Permitted Concomitant Therapy

All concomitant medications (from Visit 1) taken while the participant is in the study will be recorded. All medications needed for the health of the participant are permitted. Prohibited medications are those that may require discontinuation of IP. Participants must continue to study completion even if prohibited medications were taken.

Permitted medications are any medications required that are not specifically prohibited by the protocol during the clinical study (i.e., from Visit 1 to the end of the safety follow-up period).

### Prohibited Concomitant Medications

Prohibited medications are considered to be potentially confounding, unsafe, or both. Prohibited medications are:
Medications that should be avoided, if possible, because they could confound interpretation of the data.
Medications that may be potentially unsafe to co-administer with the IP and will lead to discontinuation of IP.

Participants who discontinue IP may remain in the study and complete all study visits and assessments (through Week 48 or 12 weeks after the last dose of IP, whichever is longer) except those assessments directly related to dosing. For analysis, the following drugs are considered to be prohibited medications. These may or may not require discontinuation of IP.

### Prohibited Medications

Any rescue medicine (see “Rescue Medicines” below).
Use of an increased dose or initiation of oral corticosteroids for general medical purposes (e.g., such as treatment of hives, poison ivy, asthma, etc.) other than use as rescue medication is prohibited in the following situations:
   Any use of dexamethasone (or other long-acting corticosteroid).
   Use of new or increased dose of baseline corticosteroid for longer than 21 cumulative days.
   Any new or increased dose of baseline corticosteroids administered after the Week 36 visit.
   Any dose that exceeds 50 mg prednisone equivalents per day.
Initiation of intravenous (IV) or intramuscular (IM) corticosteroids for general medical purposes (i.e., such as treatment of hives, poison ivy, asthma, etc.) other than use as a rescue medication is prohibited in the following situations:
   Any use of dexamethasone (or other long-acting corticosteroid).
   Use of ≥1 g of methylprednisolone (or equivalent) cumulative exposure.
   Any use of IV or IM corticosteroids after the Week 36 visit.

The following medications or interventions may require discontinuation of IP; however, participants should continue to be followed in accordance with the Table 13 with the exception of dosing, until the Week 48 visit or 12 weeks after the last dose of IP, whichever is longer, unless the participant withdraws from the study.
Cytokine or interleukin (e.g., TNF-α, IL-6, IL-12/23, IL-17, IFN, etc.) blocking therapies, complement inhibitors, abatacept, Janus kinase (JAK) inhibitors, rituximab, ocrelizumab, inebilizumab, cyclophosphamide, or tacrolimus.
IV or IM corticosteroids>1 g of methylprednisolone (or equivalent) cumulative exposure.
IV Immunoglobulin (Ig; targeted Ig for treatment or prevention of an infectious disease, such as COVID-19, is permitted).
Bone marrow, stem cell, or solid organ transplant.
Plasmapheresis, plasma exchange.
Investigational agents.
Any concomitant medication or medication dose for treatment of SS that was prohibited in protocol exclusion criteria may or may not determine continuation of IP.

### Rescue Medicines

Rescue medications are new or comprise an increase of baseline medication(s) intended to treat SS or any associated underlying rheumatologic condition. These include:
Any initiation or increase of baseline azathioprine, methotrexate (MTX), leflunomide, mycophenolate mofetil (MMF), or antimalarial (e.g., chloroquine, hydroxychloroquine, quinacrine, etc.)
Initiation of any biologic DMARD, conventional DMARD not listed, or similar systemic immune-modulating or immunosuppressive therapy
Any initiation or increase of baseline oral corticosteroid dose
Initiation of IV, IM, or IA/tendon sheath/bursal injection
Use of corticosteroids or adrenocorticotropic hormone

Use of rescue medications may result in discontinuation of IP.

Note that missing a final dose of IP does not result in treatment discontinuation.

TABLE 13
Exemplary schedule of assessments

| | Visit No. (V) | | | | | | | | | | | | | | |
|---|---|---|---|---|---|---|---|---|---|---|---|---|---|---|---|
| | | | | | | | | | | | | | | 15/ | |
| | 2 | 3 | 4 | 5 | 6 | 7 | 8 | 9 | 10 | 11 | 12 | 13 | 14 | EDV^{a} | 16^{b} |
| | Week | | | | | | | | | | | | | | |
| | 0 | 2 | 4 | 8 | 12 | 16 | 20 | 24 | 28 | 32 | 36 | 40 | 44 | 48 | 56 |
| | Study day | | | | | | | | | | | | | | |
| | | 15 − 3 d | 29 ± | 57 ± | 85 ± | 113 ± | 141 ± | 169 ± | 197 ± | 225 ± | 253 ± | 281 ± | 309 ± | 337 ± | 393 ± |
| Procedure | 1 | to +1 d | 4 d | 7 d | 7 d | 7 d | 7 d | 7 d | 7 d | 7 d | 7 d | 7 d | 7 d | 7 d | 7 d |
| ESSPRI | X | | X | X | X | X | X | X | X | X | X | X | X | X | |
| PROMIS-Fatigue SF | X | | X | X | X | X | X | X | X | X | X | X | X | X | |
| 10a | | | | | | | | | | | | | | | |
| Location of dryness | | | | | | | | X | | | | | | X | |
| improvement items | | | | | | | | | | | | | | | |
| (post-baseline) | | | | | | | | | | | | | | | |
| Subject diary | X | | | | X | | | X | | | X | | | X | |
| completion, DASPRIc | | | | | | | | | | | | | | | |
| SF-36v2 questionnaire | X | | | | X | | | X | | | X | | | X | |
| EQ-5D-5L | X | | | | X | | | X | | | X | | | X | |
| PGIC (overall SS | | X | | | X | | | X | | | X | | | X | |
| symptoms, dryness, | | | | | | | | | | | | | | | |
| pain, fatigue) | | | | | | | | | | | | | | | |
| PGIS (overall SS | X | | | | X | | | X | | | X | | | X | |
| symptoms, dryness, | | | | | | | | | | | | | | | |
| pain, fatigue) | | | | | | | | | | | | | | | |
| FSFI | X | | | | X | | | X | | | X | | | X | |
| SARS-CoV-2 rapid | X | | | | | | | | | | | | | | |
| test ^{D} | | | | | | | | | | | | | | | |
| Concomitant | X | X | X | X | X | X | X | X | X | X | X | X | X | X | X |
| medications | | | | | | | | | | | | | | | |
| Concomitant | X | X | X | X | X | X | X | X | X | X | X | X | X | X | X |
| procedures | | | | | | | | | | | | | | | |
| Vital signs (BP, HR, | X | X | X | X | X | X | X | X | X | X | X | X | X | X | X |
| RR, and temperature) | | | | | | | | | | | | | | | |
| Weight | X | | X | X | X | X | X | X | X | X | X | X | X | X | |
| Full physical exam | X | | | | | | | X | | | | | | X | |
| Symptom-driven | | X | X | X | X | X | X | | X | X | X | X | X | | X |
| physical exam | | | | | | | | | | | | | | | |
| Physical exam for | X | | X | X | X | X | X | X | X | X | X | X | X | X | |
| ESSDAI score | | | | | | | | | | | | | | | |
| 28-joint assessment | X | | X | X | X | X | X | X | X | X | X | X | X | X | |
| (TJC and SJC)G | | | | | | | | | | | | | | | |
| Unstimulated salivary | X | | | | X | | | X | | | X | | | X | |
| flow measurement | | | | | | | | | | | | | | | |
| Stimulated salivary | X | | | | X | | | X | | | X | | | X | |
| flow measurement | | | | | | | | | | | | | | | |
| ESSDAI | X | | X | X | X | X | X | X | X | X | X | X | X | X | |
| Physician Global | X | | X | X | X | X | X | X | X | X | X | X | X | X | |
| Impression of Severity | | | | | | | | | | | | | | | |
| Electrocardiogram | X | | | | | | | X | | | | | | X | |
| Safety lab tests | X | X | X | X | X | X | X | X | X | X | X | X | X | X | X |
| Urine pregnancy test | X | X | X | X | X | X | X | X | X | X | X | X | X | X | X |
| in women of | | | | | | | | | | | | | | | |
| childbearing potential | | | | | | | | | | | | | | | |
| Urinalysis | X | X | X | X | X | X | X | X | X | X | X | X | X | X | X |
| Urine immunofixation | X | | X | X | X | X | X | X | X | X | X | X | X | X | |
| and urine | | | | | | | | | | | | | | | |
| protein/creatinine ratio | | | | | | | | | | | | | | | |
| Coagulation tests | X | | | | X | | | X | | | X | | | X | X |
| (prothrombin time, | | | | | | | | | | | | | | | |
| PTT, INR) | | | | | | | | | | | | | | | |
| Autoantibody panel ^{K} | | | | | X | | | X | | | X | | | X | |
| Rheumatoid factor | X | | X | X | X | X | X | X | X | X | X | X | X | X | |
| Inflammatory markers ^{L} | X | X | X | X | X | X | X | X | X | X | X | X | X | X | |
| Serum C3, C4, free | X | | X | X | X | X | X | X | X | X | X | X | X | X | |
| light chains | | | | | | | | | | | | | | | |
| immunofixation, and | | | | | | | | | | | | | | | |
| cryoglobulins | | | | | | | | | | | | | | | |
| Exploratory flow | X | X | X | | X | | | X | | | | | | X | |
| cytometry sample | | | | | | | | | | | | | | | |
| (whole blood) ^{m, n} | | | | | | | | | | | | | | | |
| Exploratory biomarker | X | X | X | | X | | | X | | | X | | | X | |
| sample (serum) ^{o} | | | | | | | | | | | | | | | |
| Exploratory biomarker | X | X | X | | X | | | X | | | | | | X | |
| sample (plasma) ^{o} | | | | | | | | | | | | | | | |
| PBMC collection | X | X | X | | X | | | X | | | | | | X | |
| (whole blood) ⁿ | | | | | | | | | | | | | | | |
| Saliva for protein | X | | | | X | | | X | | | | | | X | |
| biomarkers | | | | | | | | | | | | | | | |
| ADA (plasma)p | X | | | | X | | | X | | | X | | | | X |
| RNA PAXgene | X | X | X | | X | | | X | | | | | | X | |
| (whole blood) | | | | | | | | | | | | | | | |
| Pharmacokinetics | Xg | | | | Xg | | | Xg | | | Xg | | | X | X |
| (plasma) | | | | | | | | | | | | | | | |
| Schirmer's test | X | | | | X | | | X | | | X | | | X | |
| (optional in a subset of | | | | | | | | | | | | | | | |
| participants) r | | | | | | | | | | | | | | | |
| Ultrasound imaging of | X | | | | | | | | X | | | | | X | |
| joints | | | | | | | | | | | | | | | |
| Salivary gland | X | | | | | | | | X | | | | | X | |
| ultrasound imaging | | | | | | | | | | | | | | | |
| Randomization | X | | | | | | | | | | | | | | |
| Administration of IP | X | X | X | X | X | X | X | X | X | X | X | X | X | X | |

ADA = anti-drug antibodies; AE = adverse event; BP = blood pressure; C = complement; d = day; DASPRI = Diary for Assessing Sjögren's Patient Reported Index; EDV = early discontinuation visit; EULAR = European Alliance of Associations for Rheumatology; ESSDAI = EULAR Sjögren's Syndrome Disease Activity Index; ESSPRI = EULAR Sjögren's Syndrome Patient Reported Index; FSFI = Female Sexual Functioning Index; HR = heart rate; Ig = immunoglobulin; INR = international normalized ratio; IP = investigational product; OLE = open-label extension; PBMC = peripheral blood mononuclear cells; PGIC = Patient Global Impression of Change; PGIS = Patient Global Impression of Severity; PROMIS-Fatigue SF-10a = Patient-Reported Outcomes Measurement Information System Fatigue-Short Form 10a PTT = partial thromboplastin time; RR = respiratory rate; SAE = serious adverse event; SARS-CoV-2 = severe acute respiratory syndrome coronavirus 2; SF-36v2 = 36-Item Short Form Survey version 2; SJC = swollen joint count; TJC = tender joint count; V = Visit; VAS = visual analog scale.
Note:
All laboratory sample collections and assessments on dosing day may be performed predose, unless specified otherwise.
i. Final visit for participants who enrolled and were dosed in the OLE.
j. Final study visit for all participants who do not enroll or do not receive at least one dose in the OLE study.
^{K} During the treatment period, the diary should be completed daily for at least 10 days prior to each visit indicated in the SoA, and every day between Week 44 and Week 48.
^{L} To participate in study, individuals require a negative SARS-CoV-2 viral test on the day of randomization.
^{m} Vital signs (during and after dosing) and observation (after dosing): participants should be followed for 1 hour post-IP administration after the first 3 dosing visits where the participant receives the IP infusion, and 0.5 hours post-IP administration at all subsequent dosing visits. Vital signs (BP, HR, RR, and temperature) should be obtained in a seated position before infusion, 10 ± 5 minutes after start of infusion, every 30 ± 10 minutes (relative to start of infusion) during infusion, and 30 ± 10 minutes after infusion plus, for the first 3 doses, before discharge from clinic. Vital signs will be monitored until stable if an immediate hypersensitivity or infusion reaction occurs.
ⁿ If no symptoms are present, the exam can be limited to assessments required for ESSDAI and total joint count., unless not required (Visit 3).
^{o} To be performed by an appropriately trained, independent assessor.
pUnstimulated salivary flow measurement should always occur before stimulated salivary flow measurement.
q. If electrocardiogram performed after phlebotomy, separate by 10 minutes.
r. Serum chemistry and hematology.
s. Anti-SSA (Ro), anti-SSB (La), and antinuclear antibody.
t. Plasma immunoglobulin levels (IgM, IgG, IgA), beta-2 microglobulin, and high-sensitivity C-reactive protein.
u. Participating sites to be determined by Sponsor.
v. Visits requiring blood sample collection for PBMC and flow cytometry should be scheduled on Monday, Tuesday, or Wednesday, due to the stability of samples. If the baseline PBMC/flow cytometry sample is not collected or is cancelled/non-evaluable for any reason, no additional blood samples for these analyses should be collected at subsequent visits.
w. Exploratory biomarkers including but not limited to chemokine (C-X-C motif) ligand 13 (CXCL13) and soluble cluster of differentiation 40 ligand (sCD40L).
x. Plasma samples for immunogenicity (ADA to dazodalibep) to be obtained prior to IP administration.
y. Plasma sample for PK to be collected pre- and postdose (within 15 minutes after infusion completion). On V15 (Week 48) (for participants not rolling into OLE) and V16 (Week 56), only one PK sample will be collected. (no dosing at these visits for participants not rolling into OLE). For participants rolling into OLE, a predose sample (relative to OLE first dose) will need to be collected from the V15 kit and postdose PK will need to be collected from the OLE Day 1 kit.
z. Performed at sites with participants willing to administer this test.
aa. Telephone-based interview for a subset of participants at their final study visit in the treatment period (Visit 15, Week 48) to discuss study participation, Unless a participant withdraws consent or is lost to follow-up, all participants should be followed through per-protocol study completion, which is Week 48 for those enrolling in the OLE study and Week 56 for those not enrolling in the OLE study.

### Assessments

### ESSPRI

The ESSPRI is a self-evaluation tool that was developed in a multicenter international cohort of 230 patients (Seror et al, 2011). The ESSPRI uses a 0 to 10 numerical rating scale (with 0 indicating no symptoms), one for the assessment of each of the 3 domains: dryness, fatigue, and pain (articular and/or muscular). The weights of the domains are identical and the mean of the scores of the 3 domains represents the final score. The recall period is stated in each question as “the last 2 weeks”. Sjögren's syndrome patients with exocrine dysfunction and severe subjective symptoms can be defined by an ESSPRI score of ≥5, which is considered as the cut-off point for “unsatisfactory symptom state” (Seror et al, 2016). The MCII in ESSPRI score is defined as a decrease of at least one point or 15% (Seror et al, 2016).

### ESSPRI [1.5] Response

The ESSPRI [1.5] refers to an at least 1.5-point reduction from baseline in ESSPRI score. Because improvements in continuous variables, such as the ESSPRI, can be difficult to interpret at the individual patient level, additional secondary outcome measures will be used to support efficacy evaluation of dazodalibep in SS. A key secondary outcome measure in this study includes the proportion of participants achieving ESSPRI [1.5] response.

### ESSPRI Fatigue

Fatigue has been shown to be one of the 2 main predictors of poor QoL in SS patients (along with pain), independent of overall disease activity and other socio-epidemiological parameters (Dias et al, 2021). A recently published analysis of the results of the National Sjögren's Foundation survey, reiterated that the one of the top 3 symptoms or signs that SS patients hope that new treatments will address is fatigue (McCoy et al, 2022).

### Diary for Assessing Sjögren's Patient Reported Index (DASPRI)

The sponsor-developed DASPRI is a participant-completed questionnaire to measure the severity of core symptoms in patients with SS. It has a recall period of 24 hours. Patients rate the severity of each symptom “at their worst” in three domains: dryness, fatigue (feeling of tiredness) and pain (joint or muscular pain in the arms or legs), using a NRS (ranging from 0 to 10, with 0 indicating no symptoms). The dryness domain assesses the severity of location-specific dryness (mouth, eyes, skin, and genital). In addition, participants will be asked to rank their the most bothersome dryness locations. Endpoints based on the DASPRI are defined as the average daily scores over a 7 day period. The DASPRI will be completed by the patient via electronic diary daily during screening, and for 10 days before Visit 6 (Week 12), Visit 9 (Week 24), and Visit 12 (Week 36), and every day between Visit 14 (Week 44) and Visit 15 (Week 48) or early discontinuation visit.

36-item Short Form Survey

The SF-36 (acute recall) is a 36-item general health status assessment that captures information about 8 health domains: Physical Functioning, Role Physical, Bodily Pain, General Health, Vitality, Social Functioning, Role Emotional, and Mental Health. The SF-36 provides scores for each domain as well as 2 psychometrically based summary scores: Physical Component Summary (PCS) and Mental Component Summary (MCS). The recall period for the acute version is one week (i.e., “last week”). The SF-36 is a validated QOL patient-reported outcome (PRO) that encompasses multiple domains and is sensitive to change (Hemingway et al, 1997). The SF-36 PCS is derived from multiple physical domains and has been shown to be validated and responsive in related autoimmune diseases (Kosinski et al, 1999, Devilliers et al, 2015). In particular, the SF-36 PCS and MCS can comprehensively capture improvements in physical activity and mental functioning, as these scores are based on the assessment of several symptoms proximal to patients with SS such as pain and mental and physical health (Sjögren's Foundation, 2021).

### ESSDAI

The ESSDAI is a systemic disease activity index that includes organ-by-organ definitions of disease activity (Seror et al, 2010). The ESSDAI grades disease activity in 12 domains (cutaneous, respiratory, renal, articular, muscular, peripheral nervous system, central nervous system, hematological, glandular, constitutional, lymphadenopathic, and biological). The weights of each domain were obtained by multiple regression modeling, using the Physician's Global Assessment of Activity as gold standard. Each domain is weighted from 1 (Biologic domain) to 6 (Muscular domain) and has 3 or 4 levels of activity per domain, ranging from 0 (no activity) to 3 or 4 (severe activity). The theoretical range of values for the ESSDAI is 0 to 123, with the final score being calculated as follows: Final score=Sum of all 12 domain scores. Domain score=Activity level×Domain weight. Low activity status is defined as ESSDAI<5, moderate activity as 5≤ESSDAI≤13, and severe activity as ESSDAI≥14 (Seror et al, 2016). ESSDAI will be performed by qualified study Investigators or sub-Investigators after successfully completing the ESSDAI training.

### Tender and Swollen Joint Counts

Arthralgia, morning stiffness, or synovitis have been reported to occur in over 50% of patients with pSS, with about 16% experiencing frank non-erosive arthritis (Ramos-Casals et al, 2015). Additionally, 7% to 17% of RA patients have a diagnosis of sSS (Young et al, 2000, Carmona et al, 2003). The 28-joint assessment will assess the following joints for tenderness and swelling: left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities, and left and right knee of the lower extremities. Each of the 28 joints will be evaluated for the presence of synovitis. At the start of the 28-joint count (prior to assessment of tenderness and swelling), participants will be asked if they have experienced or are experiencing pain in any of the 28 joints. Tender and swollen joint counts are widely used in clinical trials of rheumatologic disorders and are appropriate for use in this study because of the frequent involvement of joints in SS. Tender and swollen joint counts will be performed by an independent and appropriately trained assessor.

### PROMIS Fatigue Short Form 10a

The PROMIS-Fatigue Short Form-10a is a modified, shortened version of the FACIT-Fatigue, which includes 10 items assessing individual components of patients' experience of fatigue. Each item is rated using a 5-point scale (“not at all”, “a little bit”, “somewhat”, “quite a bit”, or “very much”), assessing their severity “during the past 7 days”. The PROMIS-Fatigue SF-10a is a validated measure that measures aspects of fatigue that are relevant to patients with autoimmune disease, such as RA (DDT COA #000015; Griffiths et al, 2022). The PROMIS-Fatigue SF-10a outcome will be scored by Health Measures Scoring Service powered by Assessment Center.

### Location of Dryness Improvement Items

The Location of Dryness Improvement Items is a series of Sponsor-developed questions assessing location of dryness. Question 1 is to be completed during screening and asks participants to rank locations of dryness most important to them to improve (1=most important location to improve, 4=least important location to improve). Questions 2 and 3 ask participants to pick the location that has improved the most (if any) at 2 different time points: Week 24 (Question 2) and Week 48 (Question 3).

### Patient Global Impression of Severity (PGIS)

The PGIS is a single item questionnaire designed to capture the participant's perception of the severity of the worst overall SS symptoms (for example, pain, fatigue, dryness) over the last 7 days on a 5-point categorical response scale (none, mild, moderate, severe, or very severe). Note, participants will receive three symptom-specific PGIS items (pain, fatigue, and dryness), as well as an overall symptom PGIS.

### Patient Global Impression of Change (PGIC)

The PGIC is a single item questionnaire designed to capture the participant's perception of change of the overall change in their SS symptoms (for example: pain, fatigue, dryness) from starting the study. Change is captured using a 5-point scale (much better, a little better, no change, a little worse, or much worse). Note, participants will receive 3 symptom-specific PGIC items (pain, fatigue, and dryness), as well as an overall symptom PGIC.

### Unstimulated Salivary Flow

The unstimulated salivary flow assessment should be performed prior to the stimulated salivary flow collection. Participants receiving standard of care for xerostomia at screening must discontinue use of pilocarpine or cevimeline for at least 12 hours and artificial saliva for at least 3 hours prior to saliva collection. Participants should be prohibited from eating or drinking for at least 90 minutes prior to saliva collection. For the unstimulated salivary flow collection, participants need to refrain from swallowing or speaking throughout the collection time, with the exception of a single swallow, immediately prior to the initiation of the timing of the collection. The total duration of the procedure is 5 minutes, during which the participants will be asked to allow saliva to accumulate in the oral cavity for a period of 60 seconds prior to emptying into a pre-weighed container. This is to be repeated a total of 5 times during the test. The weight of the container needs to be recorded prior to the initiation and at the end of the procedure.

### Stimulated Salivary Flow

Whole stimulated salivary flow will be measured to objectively assess functional changes in the salivary glands during treatment with dazodalibep. Participants receiving standard of care for xerostomia at screening must discontinue use of pilocarpine or cevimeline for at least 12 hours and artificial saliva for at least 3 hours prior to saliva collection. Participants should be prohibited from eating or drinking for at least 90 minutes prior to saliva collection. To minimize diurnal variation, every attempt should be made to collect saliva at the same time of the day as the Day 1 assessment across all subsequent visits. For whole stimulated salivary flow rate measurements, an approximately 5×5 cm parafilm square is rolled and given to the participants to be chewed like a gum at a rate of approximately 60 strokes per minute. The participant should be seated upright with eyes open and head tilted slightly forward and start chewing the parafilm for 60 seconds, at which point all the collected saliva should be spit in an extra (not pre-weighed) falcon tube, keeping the parafilm in the mouth. This first collection accustoms the participant to the procedure. Three rounds of salivary collection, each after 20 seconds of chewing, follow in a pre-weighed falcon tube. Those rounds are continuous, but timing should be stopped during the collection of the saliva and restart immediately after the saliva deposition in a pre-weighed falcon tube, for a total stimulation time of 60 seconds. If collection for 60 seconds is not feasible due to participants' inability, the total collection time should be recorded. Total stimulated saliva collected is assessed by subtracting the weight of the tube prior to collection from the final weight of the tube.

### Physician Global Impression of Severity (MDGIS)

The MDGIS represents the Investigator's overall assessment of SS disease severity and is a 5-point categorical response scale (none, mild, moderate, severe, or very severe). Every attempt should be made to have the same Investigator complete this assessment for each participant throughout the study. The MDGIS assessment should be completed on the day of the participant's visit but may be updated when labs become available.

### Female Sexual Function Index

The Female Sexual Function Index (FSFI) was designed to define female sexual function in clinical and nonclinical samples by establishing clear endpoints and outcomes for female sexual function research (Rosen et al, 2000). The FSFI is 19-item, self-reported, and validated questionnaire assessing function over the past 4 weeks in the following domains: desire, arousal, lubrication, orgasm, satisfaction, and pain. Each question is scored on a Likert scale of 0 (no sexual activity) or 1 (maximal dysfunction) to 5 (no sexual dysfunction). The final score is computed in the following manner: to normalize each domain based on item number, the sum of each domain score is first multiplied by a domain factor ratio (0.6 for desire; 0.3 for arousal; 0.3 for lubrication; 0.4 for orgasm; 0.4 for satisfaction; and 0.4 for pain) and then the domain scores are added to produce a final score. This questionnaire will only be completed by female participants willing and able to self-administer it.

### European Quality of Life 5-Dimension 5-Level Version (EQ-5D-5L)

The 5-domain, 5-level version of the EQ (EQ-5D-5L) is a generic PRO instrument that measures health status. It consists of a descriptive system and the EQ visual analogue scale (EQ VAS). The EQ-5D-5L descriptive system comprises of 5 dimensions of health: mobility, self-care, usual activities, pain/discomfort and anxiety/depression. For each dimension, patients select one of 5 levels of severity: no problems, slight problems, moderate problems, severe problems, and extreme problems. The EQ VAS records the patient's self-rated health on a vertical visual analogue scale from 0 to 100 where the endpoints are labeled the worst and best imaginable health respectively.

### Schirmer's Test

A Schirmer's test without local anesthesia will be performed at the specified time points in a subset of sites willing to perform the procedure and with participants who are willing to have the test. Schirmer's test measures lacrimal gland function. It utilizes calibrated strips of a non-toxic filter paper to measure the flow of tears. One end of the strip is placed within the lower eyelid. Both eyes need to be measured simultaneously. After the placement, participants are asked to keep their eyes gently closed for 5 minutes at which point the strips are removed from the eyelids and the extent of the wetting of each of the strips is recorded.

### Qualitative Patient Interviews (Exit Interviews) (Optional)

A subset of approximately 30 English-speaking participants with SS in the US and United Kingdom will participate in individual, 60-minute qualitative telephone interviews after completing their final study visit in the treatment period (V15, Week 48). The overall aim of these interviews is to understand if/how the QoL of participants and their SS-related symptoms were affected by participating in the study.

### Sjögren's Tool for Assessing Response (STAR)

The Sjögren's Tool for Assessing Response (STAR) is a composite responder index that was developed by the NECESSITY consortium, supported by an international panel of pSS experts, scientists, methodologists and patients to assess treatment efficacy based on improvement of disease activity (Seror et al, 2022). The STAR contains 5 domains: systemic activity, symptoms, lacrimal and glandular gland function, and biomarkers of auto-immune activity. The domains are differently weighted.

### Example 3—A Phase 2, Randomized, Double-blind, Placebo-controlled Study to Evaluate the Efficacy and Safety of Dazodalibep in Participants with Sjögren's Syndrome (SS)

A Phase 2, randomized, double-blind, placebo-controlled study to evaluate the efficacy and safety of dazodalibep in participants with (SS) is disclosed herein.

### Objectives and Endpoints:

Objective: The objective of this study was to characterize the safety and tolerability of multiple intravenous doses of dazodalibep in adult patients with SS and to obtain an assessment of efficacy.

Endpoints are presented in Table 14.

TABLE 14
Exemplary study endpoints
Endpoints
Primary
Change in baseline in ESSDAI at Day 169
Secondary
Change in baseline in ESSPRI at Day 169
Proportion of subjects achieving ESSDAI [3] and ESSDAI [4] responseª at Day 169
Change in baseline in FACIT-Fatigue score at Day 169
Change in baseline in OSDI at Day 169
Change in baseline in PGIS at Day 169
^{a} ESSDAI [3] and ESSDAI [4] response defined as a decrease of at least 3 or 4 points, respectively, from baseline in the ESSDAI at Day 169 without premature discontinuation from the study and without receiving rescue therapy

### Study Design

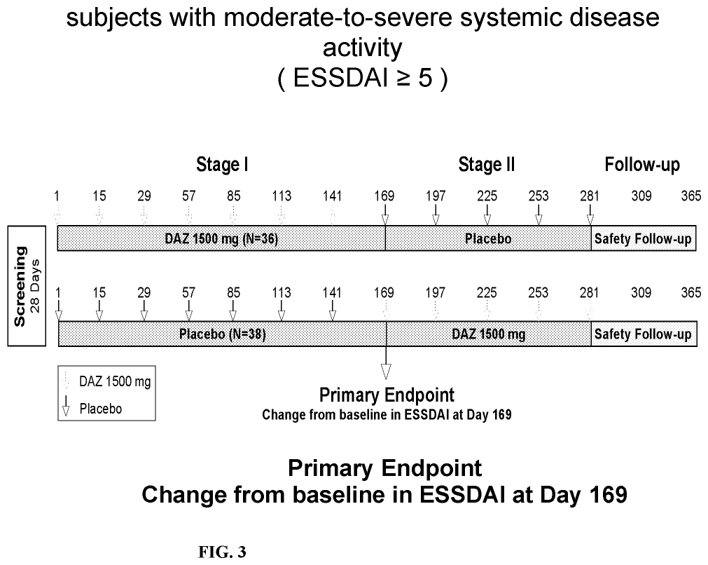

This study was a Phase 2, randomized, double-blind, placebo-controlled study to evaluate the efficacy and safety of dazodalibep in participants aged≥18 diagnosed with Sjögren's syndrome (SS) with moderate-to-severe symptom state. The study enrolled SS participants diagnosed according to the 2016 ACR/EULAR criteria, who had an ESSDAI score≥5, and who were positive for either Anti-SSA (Anti-Ro) or Rheumatoid Factor (RF), or both. The study design is provided in FIG. 3.

The study included 3 periods: screening (4 weeks), treatment period (40 Weeks) and follow-up period (12 weeks). In the treatment period, participants were randomized at 1:1 ratio to receive intravenous (IV) dose of dazodalibep or placebo (Stage I). After completion of Stage I, participants randomized to dazodalibep in Stage I received placebo and participants randomized to placebo in Stage I received dazodalibep (Stage II). Participants who had study drug discontinuation were not eligible for treatment during Stage II. All participants were followed for at least 12 weeks after their last dose of study drug administration.
Dazodalibep (Stage I), placebo (Stage II): 1500 mg dazodalibep intravenously (IV) at Day 1, Day 15, day 29, Day 57, Day 85, Day 113, and Day 141; placebo IV at Day 169, Day 197, Day 225, Day 253, and Day 281. (dazodalibep, 7 doses; placebo, 5 doses; n=36)
Placebo (Stage I), dazodalibep (Stage II): Placebo IV at Day 1, Day 15, day 29, Day 57, Day 85, Day 113, and Day 141; 1500 mg dazodalibep IV at Day 169, Day 197, Day 225, Day 253, and Day 281. (placebo, 7 doses; dazodalibep, 5 doses; n=38)

The primary objective of this study was to characterize the safety and tolerability of multiple IV doses of dazodalibep in adult patients with SS and to obtain an assessment of efficacy. This was assessed by evaluating the change from baseline in ESSDAI score at day 169.

### Study Population

### Inclusion Criteria

Adult subjects: ≥18 years of age, male or female
A diagnosis of Sjögren's according to the 2016 ACR/EULAR criteria
Have an ESSDAI score≥5 at screening
Positive for either Anti-SSA (Anti-Ro) or RF, or both at screening

### Exclusion Criteria

Medical history of confirmed deep venous thrombosis, arterial thromboembolism, or patients requiring treatment with anticoagulant drugs within the last 2 years
Concomitant polymyositis or dermatomyositis or systemic sclerosis
Positive test for, or have been treated for hepatitis B, hepatitis C, or HIV infection
Concomitant polymyositis or dermatomyositis or systemic sclerosis
Previous treatment with any biologic B-cell-depleting therapy within 12 months or other B-cell targeting therapy<3 months before randomization
Treated with systemic corticosteroids for indications other than Sjögren's, rheumatoid arthritis (RA), and systemic lupus erythematosus (SLE) for more than a total of 2 weeks within 24 weeks prior to screening visit

### Assessments

B cell subsets downstream of T cell stimulation (Ki67+ post-switch memory B cells [of CD27+/IgD−/CD19+ cells], CD27br/CD38br/IgD− plasmablasts, and CD11cbr atypical memory cells) were assayed via real time FACS throughout the study period using whole blood samples.
Serum CXCL13 concentrations, a chemokine essential for germinal center formation produced by activated follicular T cells, was measured using a qualified ELISA.
Rheumatoid factor (RF) autoantibodies were also measured using whole blood samples.

### Primary Outcomes

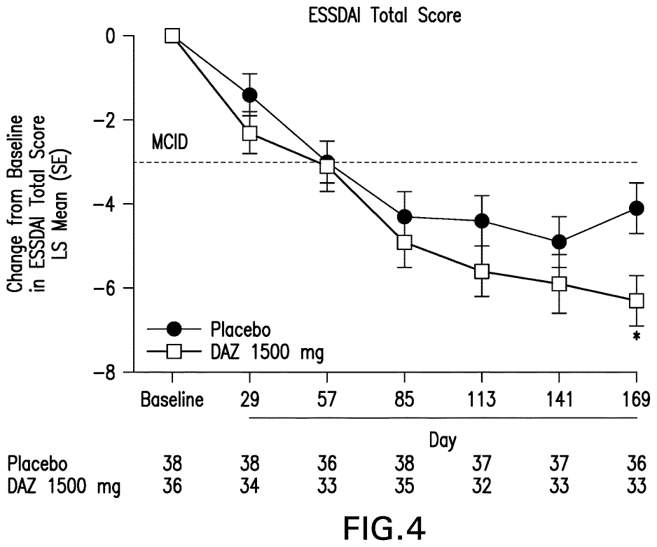

Statistically significant and clinically-meaningful changes in ESSDAI Total score versus placebo were observed in dazodalibep-treated subjects at Day 169 (FIG. 4). Summarized analyses of FIG. 4 are in Table 15.

TABLE 15
Exemplary analyses of changes from baseline in ESSDAI Total score
for dazodalibep-treated subjects on Day 169.

| Day 169 | Placebo | Dazodalibep |
|---|---|---|
| Number of subjects assessed | 36 | 33 |
| Mean change from baseline (standard error) | −4.1 (0.6) | −6.3 (0.6) |
| Mean difference from placebo | | −2.2 |
| 90% confidence interval | | (−3.6, −0.7) |
| p-value | | 0.0167 |

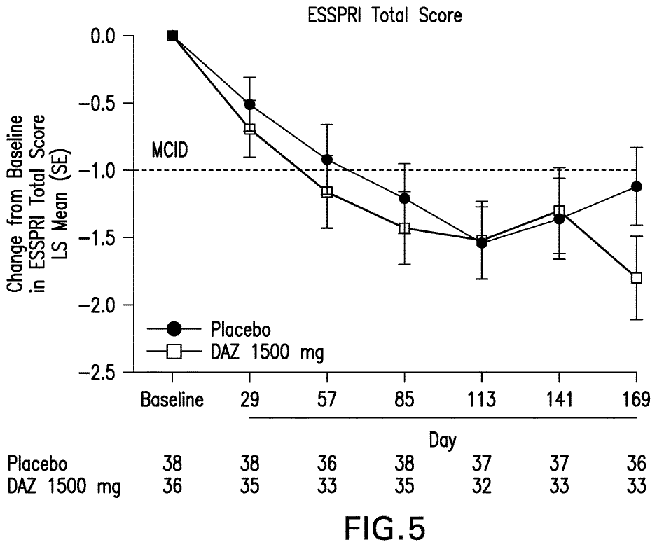

Numerically greater and clinically meaningful changes in ESSPRI Total score were observed in dazodalibep-treated subjects at Day 169 (FIG. 5). Summarized analyses of FIG. 5 are in Table 16.

TABLE 16
Exemplary analyses of changes from baseline in ESSPRI Total
score for dazodalibep-treated subjects on Day 169.

| Day 169 | Placebo | Dazodalibep |
|---|---|---|
| Number of subjects assessed | 36 | 33 |
| Mean change from baseline (standard error) | −1.12 (0.29) | −1.80 (0.31) |
| Mean difference from placebo | | −0.68 |
| 90% confidence interval | | (−1.39, 0.02) |
| p-value | | 0.1112 |

### Secondary Outcomes

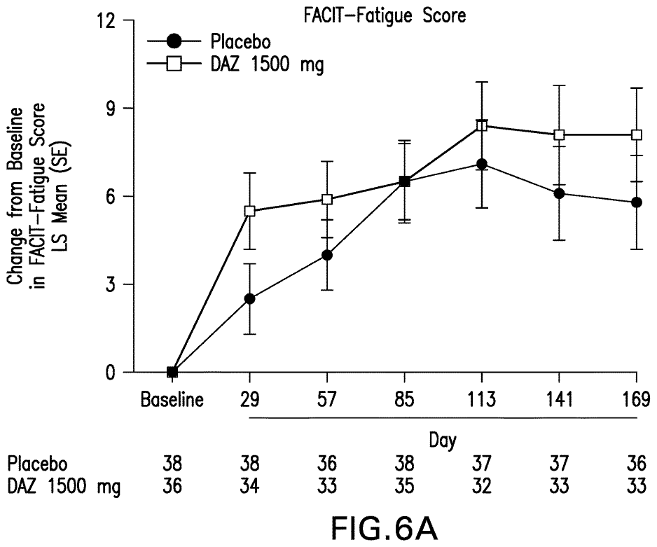

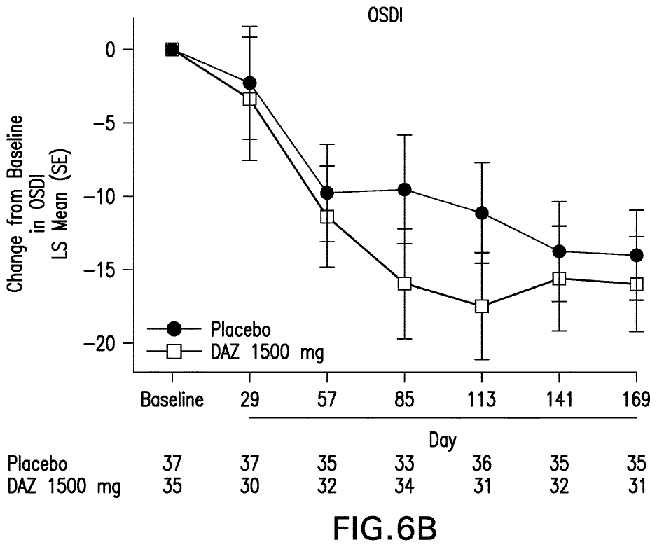

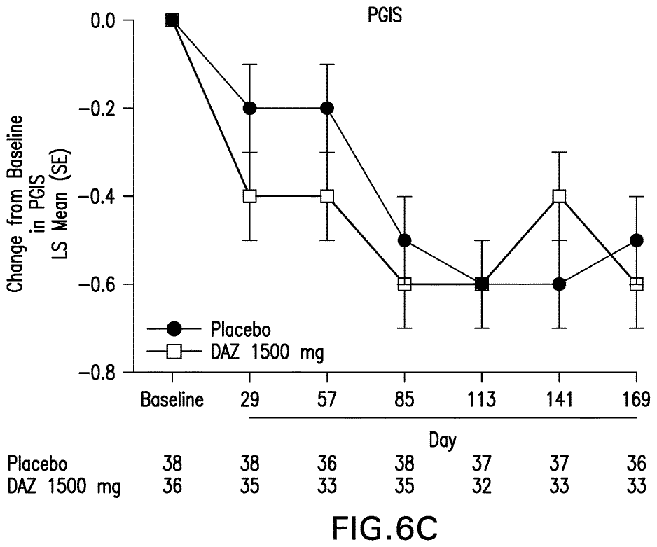

Numerically greater improvements were observed in FACIT-Fatigue Score (FIG. 6A), OSDI (FIG. 6B), and PGIS (FIG. 6C) for dazodalibep-treated subjects on Day 169. Summarized analyses for FIGS. 6A, 6B, and 6C are found in Tables 17, 18 and 19, respectively.

TABLE 17
Exemplary analyses of changes from baseline in FACIT-
Fatigue score for dazodalibep-treated subjects versus
placebo on Day 169.

| Day 169 | Placebo | Dazodalibep |
|---|---|---|
| Number of subjects assessed | 35 | 31 |
| Mean change from baseline (SD/SE/SEM) | 5.8 (1.5) | 8.1 (1.6) |
| Mean difference from placebo | | 2.4 |
| 90% confidence interval | | (−1.4, 6.1) |
| p-value | | 0.2972 |

TABLE 18
Exemplary analyses of changes from baseline in OSDI score for
dazodalibep-treated subjects versus placebo on Day 169.

| Day 169 | Placebo | Dazodalibep |
|---|---|---|
| Number of subjects assessed | 35 | 31 |
| Day 169 | Placebo | Dazodalibep |
| Mean change from baseline (SD/SE/SEM) | −14.10 (3.05) | −16.13 (3.22) |
| Mean difference from placebo | | −2.03 |
| 90% confidence interval | | (−9.42, 5.36) |
| p-value | | 0.6489 |

TABLE 19
Exemplary analyses of changes from baseline in PGIS score
for dazodalibep-treated subjects versus placebo on Day 169.

| Day 169 | Placebo | Dazodalibep |
|---|---|---|
| Number of subjects assessed | 36 | 33 |
| Mean change from baseline (SD/SE/SEM) | −0.5 (0.1) | −0.6 (0.1) |
| Mean difference from placebo | | −0.1 |
| 90% confidence interval | | (−0.4, 0.1) |
| p-value | | 0.3456 |

Clinically-meaningful changes in ESSDAI response was observed in dazodalibep-treated subjects at Day 169. Subjects were assessed via an ESSDAI responder analysis for ESSDAI [3], ESSDAI [4], ESSDAI [5], and ESSDAI [6] criteria (Table 20).

TABLE 20
Exemplary ESSDAI response analysis for ESSDAI [3],
ESSDAI [4], ESSDAI [5], and ESSDAI [6] criteria

| ESSDAI | | | Percent | |
|---|---|---|---|---|
| Response^{a} | Placebo | Dazodalibep | Decrease^{b} | P-value |
| ESSDAI [3] | 59.5% | 72.2% | 12.7% | 0.3283 |
| ESSDAI [4] | 48.6% | 66.7% | 18.1% | 0.1823 |
| ESSDAI [5] | 35.1% | 61.1% | 26.0% | 0.0449 |
| ESSDAI [6] | 34.3% | 60.0% | 25.7% | 0.0462 |

^{a}ESSDAI [3], ESSDAI [4], ESSDAI [5] and ESSDAI [6] response defined as a decrease of at least 3, 4, 5, or 6 points, respectively, from baseline in the ESSDAI at Day 169 without premature discontinuation from the study and without receiving rescue therapy
^{b}Percent decrease in ESSDAI score from baseline to Day 169

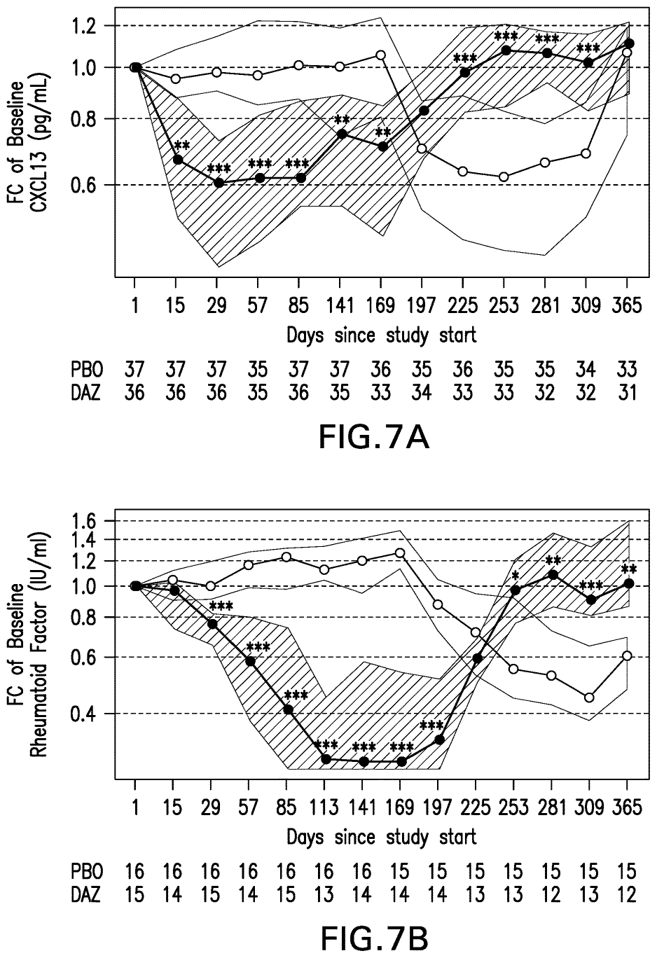

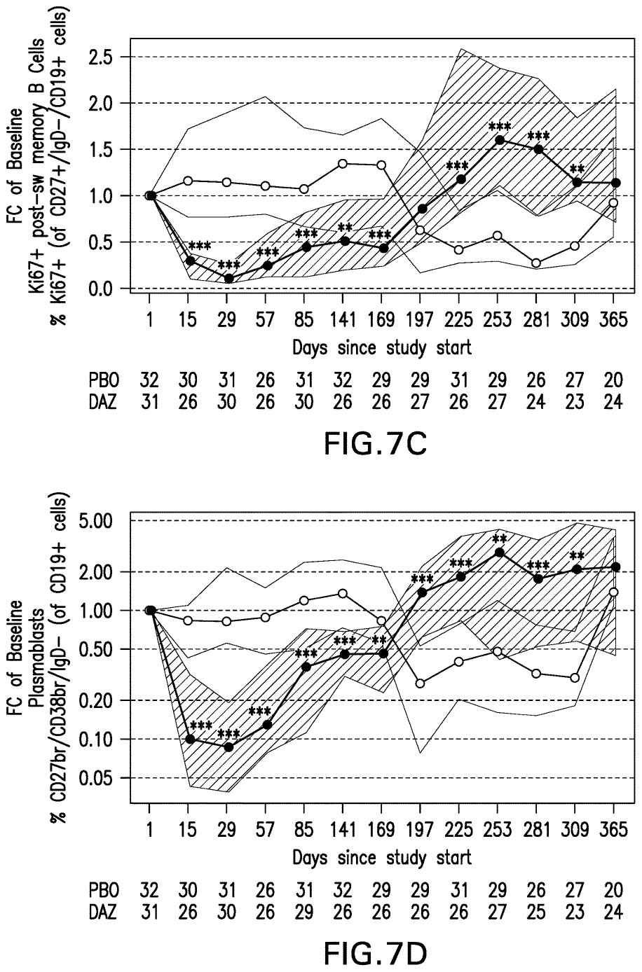

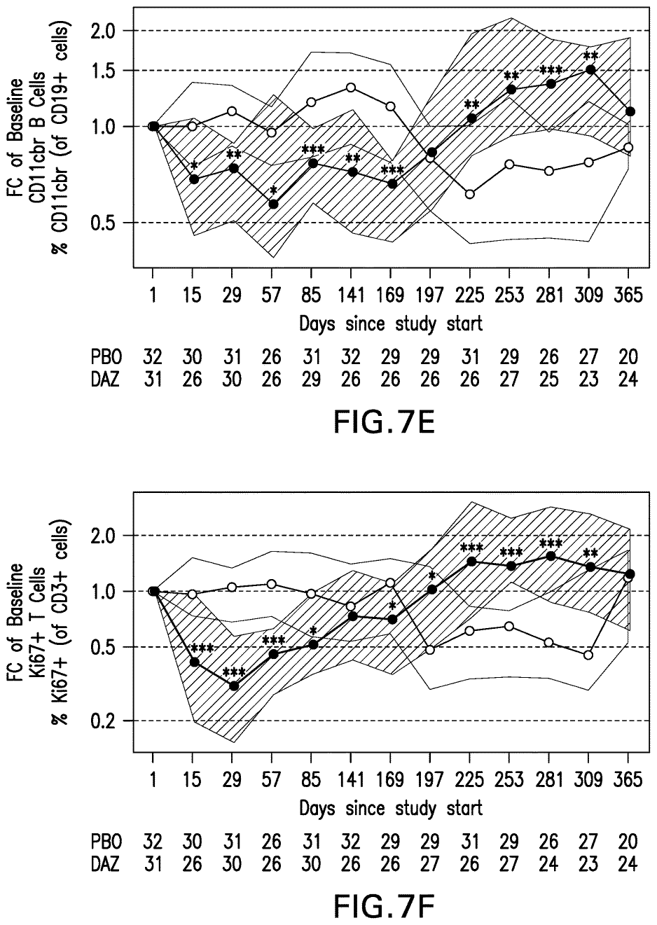

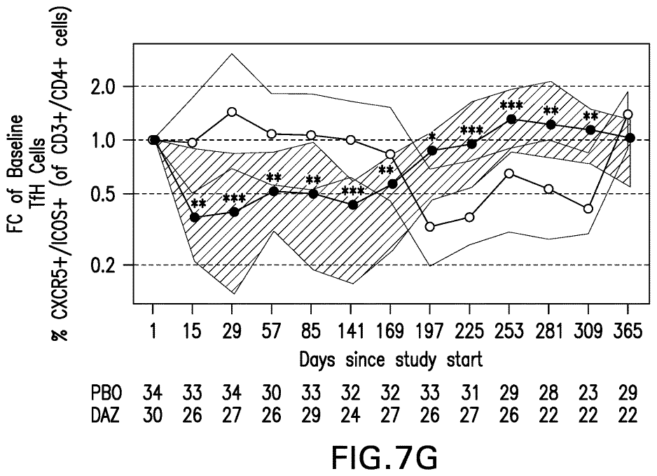

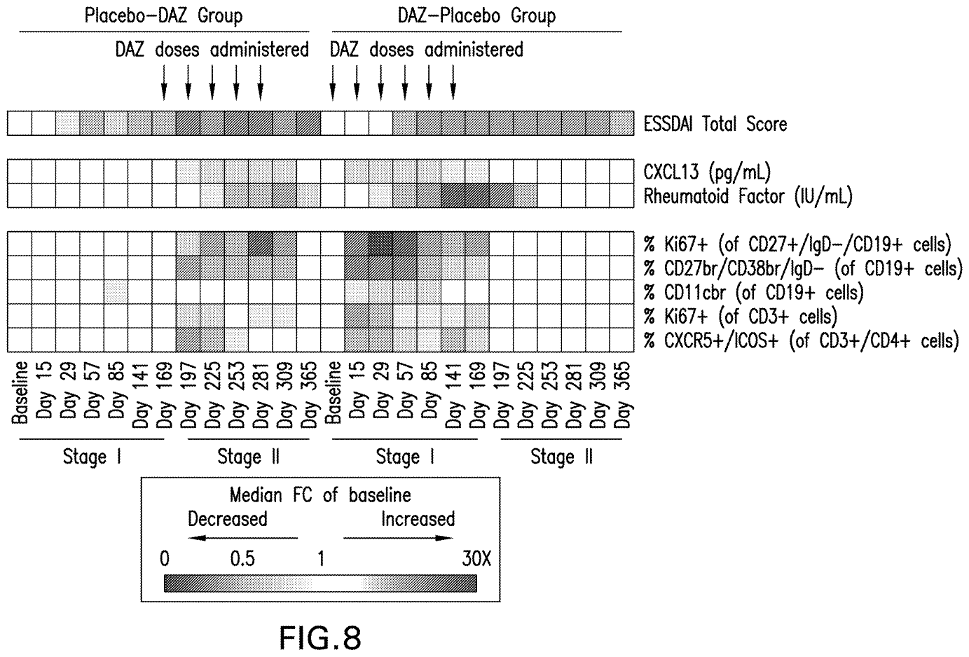

The longitudinal impact of dazodalibep on blood biomarkers of B and T cell co-stimulation in treated subjects is summarized in FIGS. 7A-7G (Stage I: pink plot; Stage II: black plot): Fold-change of baseline in CXCL13 is shown in FIG. 7A; Fold-change of baseline in RF is shown in FIG. 7B; Fold-change of baseline in Ki67+ post-switch memory B cells (percent of Ki67+ cells of identified CD27+/IgD−/CD10+ cells) is shown in FIG. 7C; Fold-change of baseline in plasmablasts (percent of CD27br/CD38br/IgD− cells of identified CD19+ cell) is shown in FIG. 7D; Fold-change in baseline in CD11cbr B cells (percent of CD11cbr+ cells of identified CD19+ cells) is shown in FIG. 7E; Fold-change of baseline in Ki67+ T cells (percent Ki67+ cells of identified CD3+ cells); Fold-change of TH cells (percent CXCR5+/ICOS+ cells of identified CD3+/CD4+ cells). Biomarkers were assessed across 365 days. A summary of these biomarker results and ESSDAI Total score are displayed as a heatmap in FIG. 8, Table 21, and Table 22.

TABLE 21
Median (+/−Interquartile Range) fold-change of baseline
in Placebo-Dazodalibep Group

| | Phase I (Placebo) | | Phase II (DAZ^{a}) | |
|---|---|---|---|---|
| Analyte Name | Day 85 | Day 169 | Day 85 | Day 169 |
| CXCL13 (pg/mL) | 1.01 (0.87, | 1.06 (0.8, | 0.62 (0.45, | 0.69 (0.53, |
| | 1.21) | 1.24) | 0.83) | 0.85) |
| Rheumatoid Factor | 1.23 (0.97, | 1.27 (1.13, | 0.55 (0.45, | 0.45 (0.38, |
| (IU/mL) | 1.31) | 1.49) | 0.92) | 0.65) |
| % Ki-67+ (of post- | 1.07 (0.67, | 1.32 (0.69, | 0.58 (0.29, | 0.47 (0.26, |
| switch memory B | 1.68) | 1.82) | 1.05) | 0.96) |
| cells) | | | | |
| % Plasmablasts (of | 1.19 (0.63, | 0.89 (0.68, | 0.58 (0.43, | 0.57 (0.33, |
| CD19 B cells) | 1.87) | 1.97) | 1.12) | 0.74) |
| % CD11 c^{high} (of | 2.37 (1.19, | 1.43 (0.95, | 1.09 (0.7, | 1.06 (0.51, |
| CD19 B cells) | 3.48) | 2.64) | 2.07) | 4.29) |
| ESSDAI Total | 0.6 (0.34, | 0.58 (0.34, | 0.41 (0.18, | 0.49 (0.25, |
| Score | 0.87) | 0.84) | 0.66) | 0.71) |

DAZ: dazodalibep

TABLE 22
Median (+/−Interquartile Range) fold-change of baseline
in Dazodalibep-Placebo Group

| | Phase I (DAZ^{a}) | | Phase II (Placebo) | |
|---|---|---|---|---|
| Analyte Name | Day 85 | Day 169 | Day 85 | Day 169 |
| CXCL13 (pg/mL) | 0.62 (0.55, | 0.71 (0.48, | 1.08 (0.84, | 1.02 (0.78, |
| | 0.89) | 0.85) | 1.21) | 1.15) |
| Rheumatoid Factor | 0.41 (0.27, | 0.29 (0.27, | 0.97 (0.77, | 0.91 (0.81, |
| (IU/mL) | 0.74) | 0.54) | 1.21) | 1.32) |
| % Ki-67+ (of post- | 0.45 (0.16, | 0.44 (0.25, | 1.59 (1.1, | 1.14 (0.94, |
| switch memory B | 0.63) | 1) | 2.36) | 2.07) |
| cells) | | | | |
| % Plasmablasts (of | 0.56 (0.3, | 0.61 (0.34, | 1.85 (0.53, | 1.84 (0.72, |
| CD19 B cells) | 0.72) | 0.84) | 3.11) | 3.45) |
| % CD 11 c^{high} (of | 0.65 (0.47, | 0.92 (0.56, | 1.41 (0.81, | 1.3 (0.92, |
| CD19 B cells) | 2.04) | 2.01) | 3.83) | 2.91) |
| ESSDAI Total | 0.6 (0.41, | 0.46 (0.26, | 0.5 (0.34, | 0.42 (0.16, |
| Score | 0.85) | 0.57) | 0.67) | 0.57) |

^{a}DAZ: dazodaliben

### Results

Concomitant with dazodalibep-related improvement in ESSDAI score observed in Stage I, significant and rapid reductions were observed Ki67+ post-switch memory B cells, Ki67+/CD27+ memory B cells, CD27br/CD38br/IgD-plasmablasts, CXCL13, CD11chigh memory B cells, and RF antibodies from Day 15 onwards in subjects receiving dazodalibep relative to placebo.
In Stage II, similar reductions in these biomarkers were found when placebo-treated subjects were transitioned to dazodalibep treatment.
In dazodalibep-treated subjects who transitioned to placebo in stage II, these biomarkers returned to baseline values.
Dazodalibep is a new therapy for the treatment of systemic disease activity in patients with SS. SS subjects with moderate-to-high systemic disease receiving dazodalibep experienced a statistically significant reduction in disease activity relative to placebo as measured by the improvement in ESSDAI score.

### Example 4—A Phase 2, Randomized, Double-blind, Placebo-controlled Study to Evaluate the Efficacy and Safety of Dazodalibep in Participants with Sjögren's Syndrome (SS)

A Phase 2, randomized, double-blind, placebo-controlled study to evaluate the efficacy and safety of dazodalibep in participants with (SS) is disclosed herein.

### Objectives and Endpoints:

Objective: The objective of this study was to characterize the safety and tolerability of multiple intravenous doses of dazodalibep in adult patients with SS and to obtain an assessment of efficacy.

Endpoints are presented in Table 23.

TABLE 23
Exemplary study endpoints

Endpoints
Primary
Change in baseline in ESSPRI at Day 169
Secondary
Proportion of subjects achieving ESSPRI response^{a} at Day 169
Change in baseline in FACIT-Fatigue score at Day 169
Change in baseline in OSDI at Day 169
Change in baseline in PGIS at Day 169
^{a}ESSPRI response defined as ≥1 point or 15% reduction from baseline in ESSPRI score without premature discontinuation from the study and without receiving rescue therapy.

### Study Design

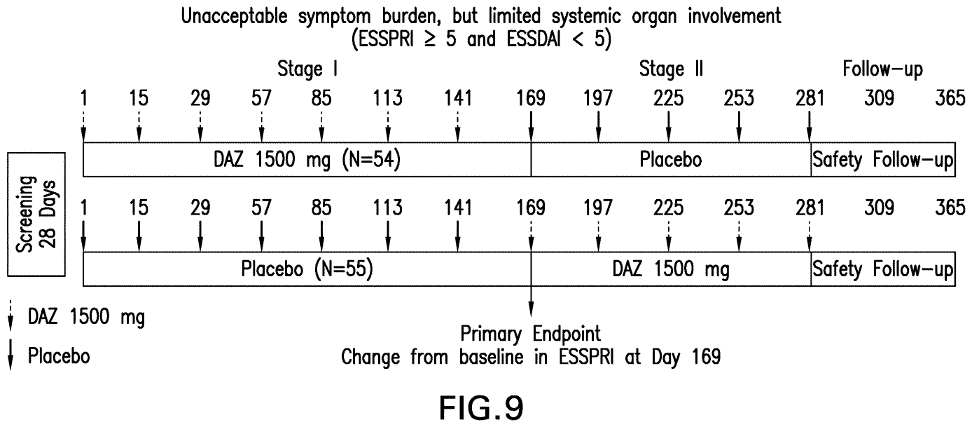

This study was a Phase 2, randomized, double-blind, placebo-controlled study to evaluate the efficacy and safety of dazodalibep in participants aged≥18 diagnosed with Sjögren's syndrome (SS) with moderate-to-severe symptom state. The study enrolled SS participants diagnosed according to the 2016 ACR/EULAR criteria, who had an ESSPRI score≥5 and an ESSDAI score<5, who were positive for either Anti-SSA (Anti-Ro) or Rheumatoid Factor (RF), or both, and who had residual salivary gland function as defined by whole stimulated salivary flow>0.1 mL/min. The study design is provided in FIG. 9.

The study included 3 periods: screening (4 weeks), treatment period (40 Weeks) and follow-up period (12 weeks). In the treatment period, participants were randomized at 1:1 ratio to receive intravenous (IV) dose of dazodalibep or placebo (Stage I). After completion of Stage I, participants randomized to dazodalibep in Stage I received placebo and participants randomized to placebo in Stage I received dazodalibep (Stage II). Participants who had study drug discontinuation were not eligible for treatment during Stage II. All participants were followed for at least 12 weeks after their last dose of study drug administration.
Dazodalibep (Stage I), placebo (Stage II): 1500 mg dazodalibep intravenously (IV) at Day 1, Day 15, Day 29, Day 57, Day 85, Day 113, and Day 141; placebo IV at Day 169, Day 197, Day 225, Day 253, and Day 281. (dazodalibep, 7 doses; placebo, 5 doses; n=54)
Placebo (Stage I), dazodalibep (Stage II): Placebo IV at Day 1, Day 15, day 29, Day 57, Day 85, Day 113, and Day 141; 1500 mg dazodalibep IV at Day 169, Day 197, Day 225, Day 253, and Day 281. (placebo, 7 doses; dazodalibep, 5 doses; n=55)

A total of 109 subjects were randomized (placebo: N=55; dazodalibep: N=54) and received study medication, with 102 (93.6%) having completed Stage I. The primary objective of this study was to characterize the safety and tolerability of multiple IV doses of dazodalibep in adult patients with SS and to obtain an assessment of efficacy. This was assessed by evaluating the change from baseline in ESSDAI score at day 169.

### Study Population

### Inclusion Criteria

Adult subjects: ≥18 years of age, male or female
A diagnosis of Sjögren's according to the 2016 ACR/EULAR criteria
Have an ESSPRI score≥5 at screening
Have an ESSDAI score<5 at screening
Positive for either Anti-SSA (Anti-Ro) or RF, or both at screening
Residual salivary gland function as defined by whole stimulated salivary flow>0.1 mL/min

### Exclusion Criteria

Medical history of confirmed deep venous thrombosis, arterial thromboembolism, or patients requiring treatment with anticoagulant drugs within the last 2 years
Concomitant polymyositis or dermatomyositis or systemic sclerosis
Positive test for, or have been treated for hepatitis B, hepatitis C, or HIV infection
Concomitant polymyositis or dermatomyositis or systemic sclerosis
Previous treatment with any biologic B-cell-depleting therapy within 12 months or other B-cell targeting therapy<3 months before randomization

### Assessments

Domain scores of ESSPRI were determined for dryness, fatigue, and pain.
The proportion of subjects achieving ESSPRI response was determined as ≥1 point or 15% reduction from baseline in ESSPRI score without premature discontinuation from the study and without receiving rescue therapy.

### Primary Outcomes

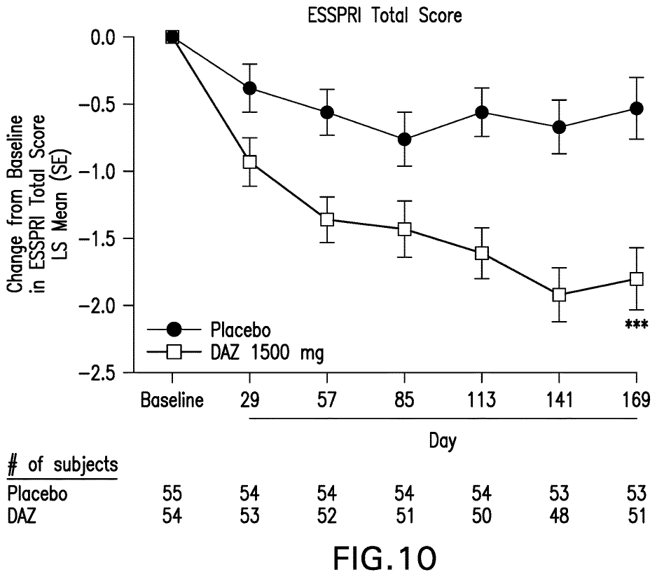

Statistically significant changes in ESSDAI Total score versus placebo were observed in dazodalibep-treated subjects at Day 169 (FIG. 10). Summarized analyses of FIG. 10 are in Table 24.

TABLE 24
Exemplary analyses of changes from baseline in ESSPRI Total
score for dazodalibep-treated subjects on Day 169.

| Day 169 | Placebo | Dazodalibep |
|---|---|---|
| Number of subjects assessed | 53 | 51 |
| Mean change from baseline | −0.53 (0.23) | −1.80 (0.23) |
| (standard error) | | |
| Mean difference from placebo | | −1.27 |
| 90% confidence interval | | (−1.82, −0.73) |
| p-value | | 0.0002 |

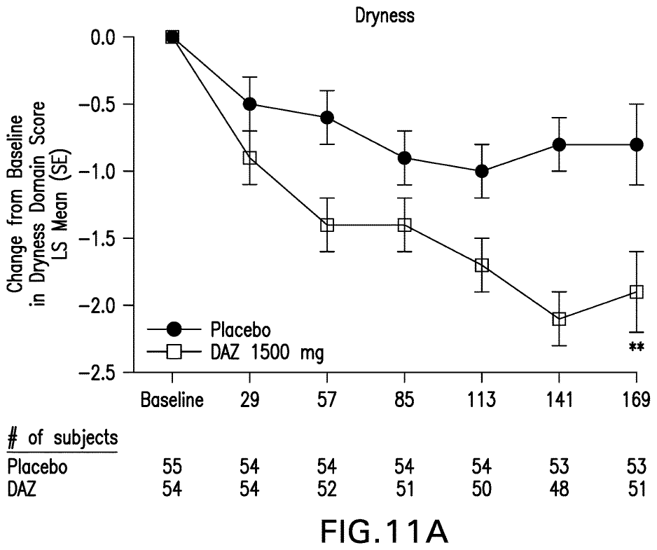

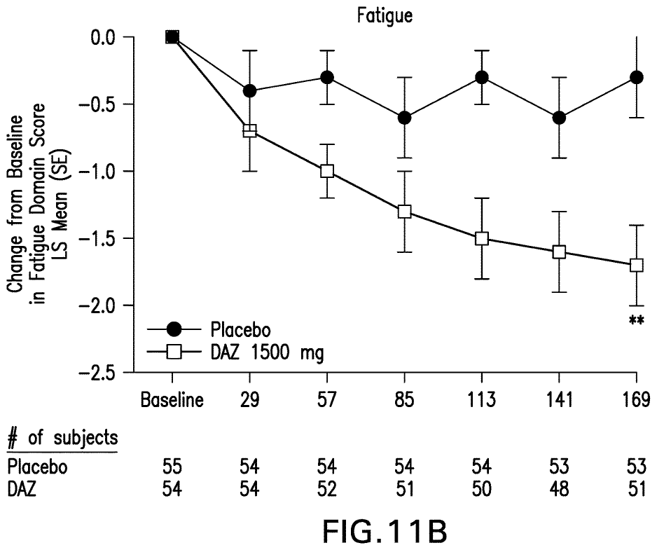

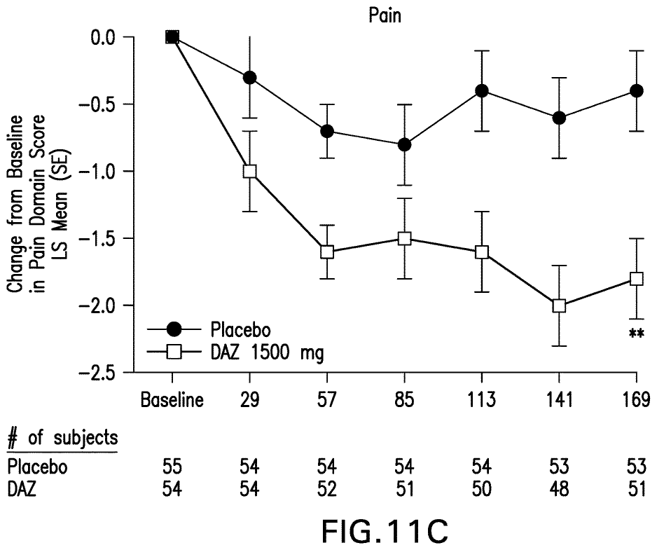

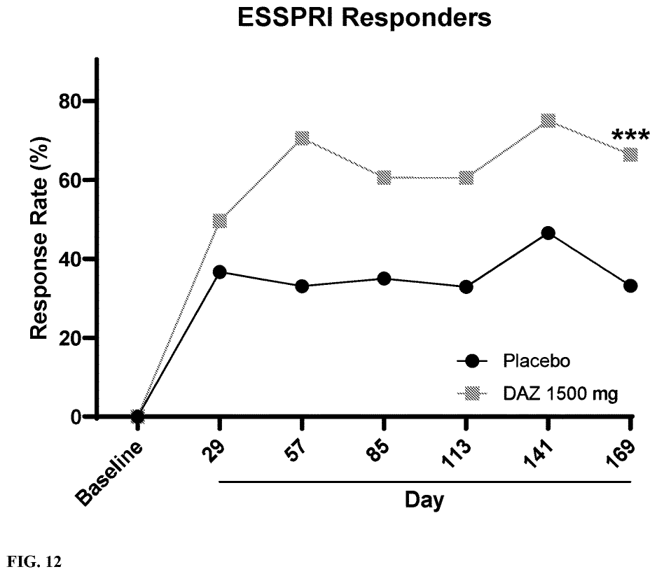

Statistically significant changes in ESSPRI Domain scores were observed in dazodalibep-treated subjects versus placebo at Day 169 for dryness (FIG. 11A), fatigue (FIG. 11B), and pain (FIG. 11C). Summarized analyses of FIGS. 11A, 11B, and 11C are in Tables 25, 26, and 27, respectively.

TABLE 25
Exemplary analyses of changes from baseline in dryness for
dazodalibep-treated subjects versus placebo on Day 169.

| Day 169 | Placebo | Dazodalibep |
|---|---|---|
| Number of subjects assessed | 53 | 51 |
| Mean change from baseline (standard error) | −0.8 (0.3) | −1.9 (0.3) |
| Mean difference from placebo | | −1.0 |
| 90% confidence interval | | (−1.7, −0.4) |
| p-value | | 0.0066 |

TABLE 26
Exemplary analyses of changes from baseline in fatigue for
dazodalibep-treated subjects versus placebo on Day 169.

| Day 169 | Placebo | Dazodalibep |
|---|---|---|
| Number of subjects assessed | 53 | 51 |
| Mean change from baseline (standard error) | −0.8 (0.3) | −1.9 (0.3) |
| Mean difference from placebo | | −1.0 |
| 90% confidence interval | | (−1.7, −0.4) |
| p-value | | 0.0066 |

TABLE 27
Exemplary analyses of changes from baseline in pain for
dazodalibep-treated subjects versus placebo on Day 169.

| Day 169 | Placebo | Dazodalibep |
|---|---|---|
| Number of subjects assessed | 53 | 51 |
| Mean change from baseline (standard error) | −0.4 (0.3) | −1.8 (0.3) |
| Mean difference from placebo | | −1.4 |
| 90% confidence interval | | (−2.1, −0.7) |
| p-value | | 0.0010 |

A statistically significant proportion of subjects achieving an ESSPRI response for dazodalibep-treated subjects versus placebo was observed on Day 169 (FIG. 12). Summarized analysis for FIG. 12 is found in Table 28.

TABLE 28
Exemplary proportion of subjects achieving an ESSPRI response
for dazodalibep-treated subjects versus placebo on Day 169.

| Day 169 | Placebo | Dazodalibep |
|---|---|---|
| Number of subjects assessed | 55 | 54 |
| Mean change from baseline (standard error) | 18 (32.7) | 36 (66.7) |
| Mean difference from placebo | | 4.0 |
| 90% confidence interval | | (2.0, 7.8) |
| p-value | | 0.0008 |

### Secondary Outcomes

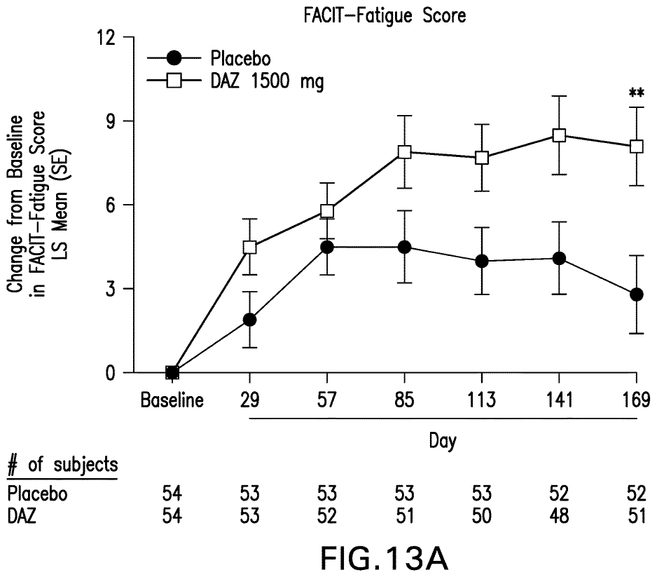

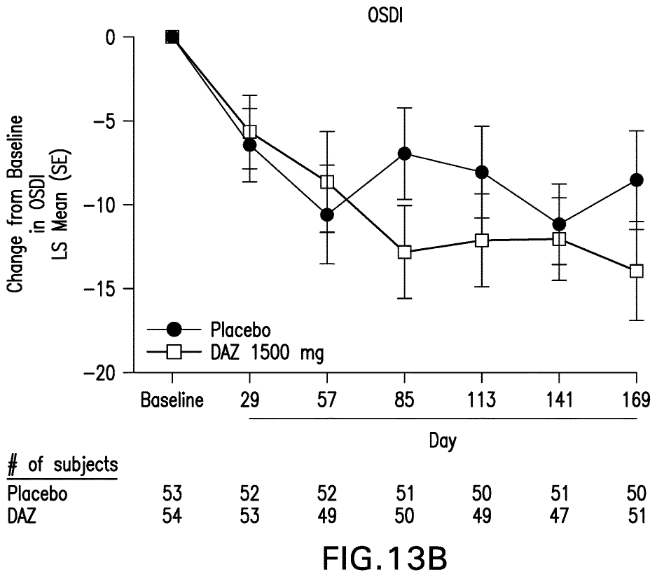

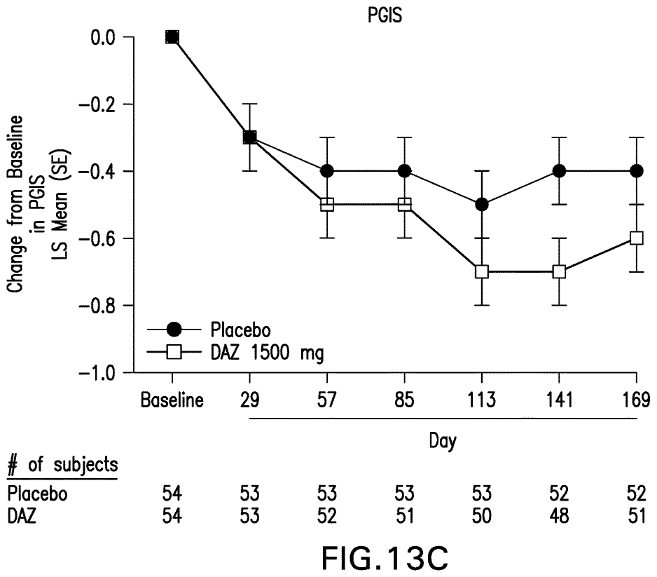

Numerically greater improvements were observed in FACIT-Fatigue Score (FIG. 13A), OSDI (FIG. 13B), and PGIS (FIG. 13C) for dazodalibep-treated subjects versus placebo on Day 169. Summarized analyses for FIGS. 13A, 13B, and 13C are found in Tables 29, 30, and 31, respectively.

TABLE 29
Exemplary analyses of changes from baseline in FACIT-Fatigue
score for dazodalibep-treated versus placebo subjects on Day 169.

| Day 169 | Placebo | Dazodalibep |
|---|---|---|
| Number of subjects assessed | 52 | 51 |
| Mean change from baseline (SD/SE/SEM) | 2.8 (1.4) | 8.1 (1.4) |
| Mean difference from placebo | | 5.3 |
| 90% confidence interval | | (2.0, 8.7) |
| p-value | | 0.0095 |

TABLE 30
Exemplary analyses of changes from baseline in OSDI score for
dazodalibep-treated versus placebo subjects on Day 169.

| Day 169 | Placebo | Dazodalibep |
|---|---|---|
| Number of subjects assessed | 50 | 51 |
| Mean change from baseline (SD/SE/SEM) | −8.52 (2.94) | −13.95 (2.95) |
| Mean difference from placebo | | −5.43 |
| 90% confidence interval | | (−12.31, 1.46) |
| p-value | | 0.1936 |

TABLE 31
Exemplary analyses of changes from baseline in PGIS score for
dazodalibep-treated versus placebo subjects on Day 169.

| Day 169 | Placebo | Dazodalibep |
|---|---|---|
| Number of subjects assessed | 52 | 51 |
| Mean change from baseline (SD/SE/SEM) | −0.4 (0.1) | −0.6 (0.1) |
| Mean difference from placebo | | −0.2 |
| 90% confidence interval | | (−0.5, 0.0) |
| p-value | | 0.1781 |

### Results

The Phase 2 trial of Dazodalibep evaluated two patient populations; the first group included patients with moderate-to-severe systemic disease activity, and the second group included those with moderate-to-severe symptomatology including dryness, fatigue and pain despite lacking additional organ involvement. Results from the trial indicate treatment with dazodalibep addresses the unmet therapeutic needs for this challenging condition, which has no approved disease-modifying therapies to date. Dazodalibep is the only investigational medicine to achieve the primary endpoint in both patient populations in a Phase 2 trial.

Results in Patients with Moderate-to-Severe Systemic Disease Activity

The first population of the Phase 2 trial included patients with moderate-to-severe systemic disease activity as defined by a EULAR Sjögren's Syndrome Disease Activity Index (ESSDAI) score of ≥5. The trial assessed changes in the ESSDAI score and other Sjögren's measures as well as safety of dazodalibep treatment compared to placebo.

The primary endpoint was achieved, and patients treated with dazodalibep experienced a statistically significant (p-value-0.0167) and clinically meaningful improvement in their disease activity (6.3-point reduction in their ESSDAI score) versus those who received placebo (4.1-point reduction) and showed positive trends in several other assessments at Day 169.

All ESSDAI responder analyses (pre-specified and post-hoc) favored dazodalibep over placebo, with greater numerical differences for the highest levels of response.

Patients treated with dazodalibep experienced numerically greater improvements in ESSPRI score and fatigue compared to those who received placebo at Day 169.

Safety profiles were similar between the groups, with the most commonly reported adverse events including COVID-19, diarrhea, dizziness, ligament sprain and upper respiratory infections.

Results in Patients with Moderate-to-Severe Symptomatology

The Phase 2 trial also evaluated a second population of patients with moderate-to-severe symptomatology including dryness, fatigue and pain despite lacking additional organ involvement as defined by a EULAR Sjögren's Syndrome Patient Reported Index (ESSPRI) score of ≥5, indicative of significant symptomatic burden, and an ESSDAI score of <5 representing limited extraglandular organ involvement.

The primary endpoint was achieved, and patients treated with dazodalibep experienced a statistically significant and clinically meaningful improvement in the key subjective symptoms of Sjögren's syndrome (1.8-point reduction in their ESSPRI score) compared to those treated with placebo (0.53-point reduction) at Day 169 (p-value=0.0002).

Statistically significant improvements were seen in patients treated with dazodalibep across the three domains of ESSPRI measuring dryness, fatigue and pain compared to those treated with placebo.
a. Patients treated with dazodalibep experienced a 1.9-point reduction in their dryness score compared to a 0.8-point reduction for those treated with placebo (p=0.0066).
b. Patients treated with dazodalibep experienced a 1.7-point reduction in their fatigue score compared to a 0.3-point reduction for those treated with placebo (p=0.0022).
c. Patients treated with dazodalibep experienced a 1.8-point reduction in their pain score compared to a 0.4-point reduction for those treated with placebo (p=0.0010).

Significantly more patients treated with dazodalibep achieved a clinically meaningful≥1-point or ≥15% reduction in ESSPRI scores compared to those treated with placebo (66.7% versus 32.7% respectively, p-value=0.0008).

Safety profiles were similar between the groups, with the most commonly reported adverse events including COVID-19, nasopharyngitis and anemia.

Results from Biomarker Analysis of Patients with Sjögren's Syndrome

The biological mechanism of dazodalibep, focusing on specific blood biomarkers that contribute to Sjögren's syndrome, was also studied in the Phase 2 trial. Overactivation of CD40 ligand-CD40 signaling between immune cells, including CD40 ligand on T cells and CD40 on B cells, contributes to the exaggerated immune responses that characterize Sjögren's. Dazodalibep is a CD40 ligand antagonist designed to block this interaction and disrupts the overactivation of the CD40 ligand co-stimulatory pathway. Significant and rapid reductions in blood biomarkers associated with T and B cell co-stimulation were observed in patients who received dazodalibep as compared to placebo.

1. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16,
wherein the Tn3 scaffold is administered at a dose of about 1500 mg once every 4 weeks,
wherein the subject has moderate-to-severe symptom state defined by ESSPRI score of ≥5, with a low systemic disease activity defined by ESSDAI score of <5, and wherein a Diary for Assessing Sjögren's Patient Reported Index (DASPRI) score is reduced in the subject following the administration.

2. The method of claim 1, wherein prior to the once every 4 week dosing, the subject was administered at least 3 loading doses of 1500 mg each.

3. The method of claim 2, wherein the at least 3 loading doses are administered at weeks 0, 2, and 4.

4. A method of treating Sjögren's syndrome (SS) in a subject in need thereof comprising: administering a Tn3 scaffold, wherein the Tn3 scaffold comprises a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16,
wherein the Tn3 scaffold is administered at a dose of about 3000 mg once every 12 weeks,
wherein the subject has moderate-to-severe symptom state defined by ESSPRI score≥5, with a low systemic disease activity defined by ESSDAI score<5, and wherein a Diary for Assessing Sjögren's Patient Reported Index (DASPRI) score is reduced in the subject following the administration.

5. The method of claim 4, wherein prior to the once every 12 week dosing, the subject was administered at least 3 loading doses of 3000 mg each.

6. The method of claim 5, wherein the at least 3 loading doses are administered at week 0, 4, and 12.

7. The method of any one of claims 1-6, wherein salivary gland function in the subject is improved following administration of the Tn3 scaffold by about 3 weeks, 1 month, 2 months, or 4 months following the administration.

8. The method of claim 7, wherein salivary gland function is determined by whole stimulated salivary flow.

9. The method of any one of claims 1-8, wherein the ESSPRI score is reduced following the administration.

10. The method of claim 9, wherein the ESSPRI score is reduced by at least about 1.5, 2, 3, 4, or 5 points.

11. The method of any one of claims 1-10, wherein the DASPRI score is reduced by 2 weeks, 4 weeks, 1 month, 2 months, 3 months, 4 months, 5 months, or 6 months following the administration.

12. The method of claim 11, wherein the DASPRI score is reduced by at least about 5, 10, 15, or 20 points.

13. The method of any one of claims 1-12, wherein the administration of the Tn3 scaffold is effective at reducing a tender and swollen joint count baseline score of the subject following the administration.

14. The method of any one of claims 1-13, wherein the administration of the Tn3 scaffold is effective at reducing a Short Form 36 (SF-36) Health Survey baseline score of the subject following the administration.

15. The method of any one of claims 1-14, wherein the administration of the Tn3 scaffold is effective at improving a baseline score of a subject, wherein the baseline scores are selected from the group consisting of: functional assessment of chronic illness therapy fatigue (FACIT-fatigue), PROMIS fatigue short form 10a, ocular surface disease index (OSDI), EQ-5D-5L, and patient global impression of severity (PGIS).

16. The method of any one of claims 1-15, wherein the administration of the Tn3 scaffold is effective at reducing a baseline level of markers of inflammation following the administration, and wherein the markers are selected from the group consisting of: immunoglobulin, β-2 microglobulin, C-reactive protein, and combinations thereof.

17. The method of any one of claims 1-16, wherein the administration of the Tn3 scaffold is effective at reducing a baseline level of a biomarker, and wherein the biomarker is selected from the group consisting of: plasma soluble CD40L, Ki67+CD27+ memory B cell, CD19, CD20, CD27, CD38, CD138, CD11c bright/high B cell, CD3, CD4, CD8, T follicular helper (Tfh) cell, serum CXCL13, rheumatoid factor, anti-SSA autoantibodies, anti-Ro autoantibodies, anti-SSB autoantibodies, anti-La autoantibodies, and combinations thereof.

18. The method of any one of claims 1-17, wherein the administration of the Tn3 scaffold is effective at reducing a baseline level of a disease symptom, and wherein the disease symptom is selected from the group consisting of: fatigue, oral dryness, ocular dryness, vaginal dryness, pain, and combinations thereof.

19. The method of any one of claims 1-18, wherein expression levels of genes, or levels of proteins encoded by the genes, associated with SS are reduced in a blood sample of the subject by week 48 following the administration.

20. The method of any one of claims 1-19, wherein levels of B cells selected from the group consisting of: CD27+ memory B cells, marginal zone B cells, plasmablasts, plasma cells, and combinations thereof, are reduced in a blood sample of the subject by week 48 following the administration.

21. The method of any one of claims 1-20, wherein the subject is positive for anti-Ro autoantibodies, rheumatoid factor (RF), or both anti-Ro autoantibodies and RF.

22. The method of any one of claims 1-21, wherein the Tn3 scaffold is administered intravenously.

23. The method of any one of claims 1-22, wherein the Tn3 scaffold comprises two CD40L-specific monomer subunits connected in tandem.

24. The method of claim 23, wherein the two CD40L-specific monomer subunits each comprise SEQ ID NO: 3.

25. The method of any of one of claims 23-24, wherein the CD40L-specific monomer subunits are connected by a linker.

26. The method of any one of claims 23-25, wherein at least one CD40L-specific monomer subunit is fused or conjugated to a polyethylene glycol (PEG) directly.

27. The method of any one of claims 23-26, wherein at least one CD40L-specific monomer subunit is fused or conjugated to a polyethylene glycol (PEG) via a linker.

28. The method of claim 27, wherein the linker comprises a peptide linker.

29. The method of claim 28, wherein the linker comprises SEQ ID NO: 7, SEQ ID NO: 8, SEQ ID NO: 9, or SEQ ID NO: 10.

30. The method of any one of claims 23-29, wherein at least one CD40L-specific monomer subunit is fused or conjugated to an albumin.

31. The method of claim 30, wherein the albumin is human serum albumin (HSA).

32. The method of claim 31, wherein the HSA is a variant HSA comprising SEQ ID NO: 4.

33. The method of any one of claims 1-32, wherein the Tn3 scaffold comprises SEQ ID NO: 1.

34. The method of any one of claims 1-33, wherein the subject has a co-existing autoimmune indication.

35. The method of claim 34, wherein the co-existing autoimmune indication is rheumatoid arthritis (RA), systemic lupus erythematosus (SLE), or both RA and SLE.

36. The method of claim 34, wherein the co-existing autoimmune indication is rheumatoid arthritis.

37. The method of claim 34, wherein the co-existing autoimmune indication is systemic lupus erythematosus.


SEQUENCE LISTING (25 sequences)

SEQ ID NO: 1 - protein, 785 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 83 aa, synthetic construct
IEVKDVTDTTALITWSDDFGEYVWCELTYGIKDVPGDRTTIDLWYHHAHYSIGNLKPDTEYEVSLICRRGDMSSNPAKETFTT

SEQ ID NO: 3 - protein, 83 aa, synthetic construct
IEVKDVTDTTALITWSDDFGEYVWCELTYGIKDVPGDRTTIDLWYHHAHYSIGNLKPDTEYEVSLICRSGDMSSNPAKETFTT

SEQ ID NO: 4 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - skipped

SEQ ID NO: 6 - protein, 5 aa, synthetic construct
ALITW

SEQ ID NO: 7 - protein, 10 aa, synthetic construct
GGGGSGGGGS

SEQ ID NO: 8 - protein, 15 aa, synthetic construct
GGGGSGGGGSGGGGS

SEQ ID NO: 9 - protein, 10 aa, synthetic construct
GGGGGGGGGG

SEQ ID NO: 10 - protein, 15 aa, synthetic construct
GGGGGGGGGGGGGGG

SEQ ID NO: 11 - protein, 7 aa, synthetic construct
KDVTDTT

SEQ ID NO: 12 - protein, 9 aa, synthetic construct
SDDFGEYVW

SEQ ID NO: 13 - protein, 7 aa, synthetic construct
KDVPGDR

SEQ ID NO: 14 - protein, 6 aa, synthetic construct
WYHHAH

SEQ ID NO: 15 - protein, 8 aa, synthetic construct
GNLKPDTE

SEQ ID NO: 16 - protein, 10 aa, synthetic construct
RSGDMSSNPA

SEQ ID NO: 17 - protein, 7 aa, synthetic construct
CELTYGI

SEQ ID NO: 18 - protein, 5 aa, synthetic construct
TTIDL

SEQ ID NO: 19 - skipped

SEQ ID NO: 20 - protein, 7 aa, synthetic construct
YEVSLIC

SEQ ID NO: 21 - protein, 6 aa, synthetic construct
KETFTT

SEQ ID NO: 22 - protein, 85 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 23 - protein, 10 aa, synthetic construct
RLDAPSQIEV

SEQ ID NO: 24 - protein, 5 aa, synthetic construct
SQIEV

SEQ ID NO: 25 - protein, 90 aa, synthetic construct
RLDAPSQIEVKDVTDTTALITWSDDFGEYVWCELTYGIKDVPGDRTTIDLWYHHAHYSIGNLKPDTEYEVSLICRSGDMSSNPAKETFTT